(19) United States
(12) Patent Application Publication
(10) Pub. No.: US 2017/0296570 A1
(43) Pub. Date: Oct. 19, 2017

(54) High Drug Load Tablets Comprising Sofosbuvir

(71) Applicant: Sandoz AG, Basel (CH)

(72) Inventors: Nolwenn Martin, Kundl (AT); Franz Schwarz, Kundl (AT); Georg Anker, Kundl (AT)

(73) Assignee: Sandoz AG, Basel (CH)

(21) Appl. No.: 15/516,968

(22) PCT Filed: Oct. 8, 2015
(86) PCT No.: PCT/EP2015/073276

§ 371 Date: Apr. 5, 2017

(30) Foreign Application Priority Data
Oct. 8, 2014 (EP) 14188205.0

(51) Int. Cl. A61K 31/7072; A61K 9/20

(52) U.S. Cl. CPC A61K 31/7072; A61K 9/2054; A61K 9/2095; A61K 9/2009; A61K 9/2018; A61K 9/2013; C07B 2200/13

(57) ABSTRACT

A tablet comprising a polymorphic form of crystalline sofosbuvir having a moisture stability of at least 95% in an amount of at least 40 weight-%.

The present invention relates to a high drug load tablet comprising a polymorphic form of sofosbuvir which is stable when exposed to moisture, and a process for the preparation of the tablet. Further, the present invention relates to the use of the tablet for the treatment of hepatitis C. Yet further, the present invention relates to the use of a polymorphic form of sofosbuvir which is stable when exposed to moisture for the preparation of a high drug load tablet.

Sofosbuvir according to formula (I)

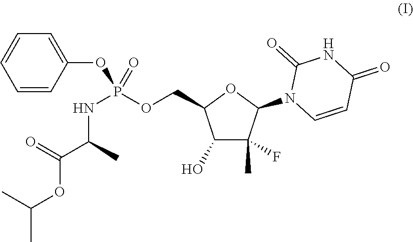 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

with IUPAC name (S)-isopropyl 2-(((S)-(((2R,3R,4R,5R)-5-(2,4-dioxo-3,4-dihydropyrimidin-1(2H)-yl)-4-fluoro-3-hydroxy-4-methyltetrahydrofuran-2-yl)methoxy)(phenoxy)phosphoryl)-amino)propanoate is a drug inhibiting the RNA polymerase used by the hepatitis C virus to replicate its RNA.

In WO 2010/135569 A, sofosbuvir is described as a moisture instable compound. In particular, it was found that under stress conditions at 40° C. and a relative humidity (RH) of 75%, sofosbuvir deliquesces after a few hours. WO 2013/082003 A describes compositions comprising sofosbuvir. Among other, capsules and tablets are disclosed.

Generally, a sufficiently fast and complete dissolution of such compositions is desired to allow for a sufficient bioavailability of the sofosbuvir. Tablets exhibiting such dissolution after 15 minutes are described in WO 2013/082003 A. These tablets contain polymorphic form 1 of sofosbuvir in an amount of 33.33 weight-%. According to the general description of WO 2013/082003 A, the maximum content of pharmaceutical compositions comprising sofosbuvir is disclosed to be 35 weight-%, and at least 65 weight-% of the tablets consist of pharmaceutically acceptable excipients.

Therefore, due to this high amount of excipients according to this teaching of the prior art, comparatively large tablets have to be prepared for administering the desired amount of sofosbuvir to a patient in need thereof, and swallowing such large tablets is uncomfortable for the patient.

Thus, an object of the present invention was the provision of tablets comprising sofosbuvir and exhibiting a sufficiently fast and complete dissolution, avoiding the disadvantages of the tablets disclosed in the prior art.

Surprisingly, although WO 2013/082003 A, on page 23, line 27, explicitly teaches that with regard to tablets, already a sofosbuvir content of 33 weight-% has to be considered as a relatively high content, it was found that tablets comprising sofosbuvir can be provided having a very high drug load when a specific polymorphic form of sofosbuvir is used. Specifically, it was found that contrary to the teaching of WO 2013/082003 A, tablets having a significantly higher sofosbuvir content can be prepared using a polymorphic form of sofosbuvir which has a specific moisture stability.

Further it has surprisingly been found that tablets comprising polymorphic form 7 of sofosbuvir of formula (I) in an amount of at least 25 weight-% have a good dissolution profile.

Therefore, the present invention relates to a tablet comprising a polymorphic form of crystalline sofosbuvir according to formula (I)

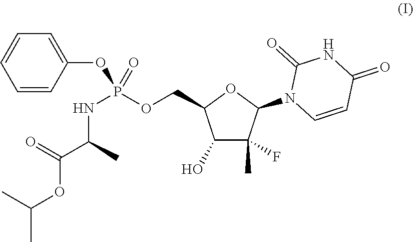 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

in an amount of at least 40 weight-%, based on the total weight of the tablet, wherein the polymorphic form has a moisture stability of at least 95%, wherein the moisture stability of the polymorphic form is defined as the amount of the polymorphic form which is present after an exposure to a relative humidity of (75±1) % at a temperature of (40±1)° C. for an exposure time of (18±1) hours, relative to the amount of the polymorphic form before said exposure, as determined via X-ray powder diffraction pattern analysis at a temperature in the range of from 15 to 25° C. with Cu-Kalpha_{1,2} radiation having a wavelength of 0.15419 nm before the exposure and after the exposure.

Preferably, the polymorphic form of sofosbuvir comprised in the tablet is substantially free of its corresponding phosphorous-5 based diastereomer (S)-isopropyl 2-(((R)-(((2R,3R,4R,5R)-5-(2,4-dioxo-3,4-dihydropyrimidin-1(2H)-yl)-4-fluoro-3-hydroxy-4-methyltetrahydrofuran-2-yl)methoxy)(phenoxy)phosphoryl)amino)propanoate. Preferably, the polymorphic form of sofosbuvir comprised in the tablet is at least 95% free from its corresponding phosphorous-based diastereomer. More preferably, the polymorphic form of sofosbuvir comprised in the tablet is at least 97% free from its corresponding phosphorous-based diastereomer. More preferably, the polymorphic form of sofosbuvir comprised in the tablet is at least 99% free from its corresponding phosphorous-based diastereomer. More preferably, the polymorphic form of sofosbuvir comprised in the tablet is at least 99.5% free from its corresponding phos-phorous-based diastereomer. More preferably, the polymorphic form of sofosbuvir comprised in the tablet is at least 99.9% free from its corresponding phosphorous-based diastereomer.

### Tablet

While tablets comprising the polymorphic form of sofosbuvir in an amount in the range of at least 36 weight-% or at least 37 weight-% or at least 38 weight-% or at least 39 weight-% are not excluded from the present invention, the preferred content is at least 40 weight-%. More preferably, the tablet comprises the polymorphic form in an amount in the range of from 40 to 95 weight-%, more preferably from 40 to 90 weight-%, more preferably from 45 to 90 weight-%, more preferably from 45 to 85 weight-%, more preferably from 45 to 80 weight-%, more preferably from 50 to 80 weight-%, based on the total weight of the tablet. More preferably, the tablet comprises the polymorphic form in an amount of from 50 to 75 weight-% based on the total weight of the tablet. Preferred ranges include, for example, a range of from 50 to 60 weight-% or a range of from 55 to 65 weight-% or a range of from 60 to 70 weight-% or a range of from 65 to 75 weight-%. More preferably, the tablet comprises the polymorphic form in an amount of ≧75 weight-% based on the total weight of the tablet. Preferred ranges include, for example, a range of from 75 to 90 weight-% or a range of from 75 to 85 weight-%, or a range of from 75 to 80 weight-% or a range of from 80 to 90 weight-% or a range of from 85 to 90 weight-% based on the total weight of the tablet.

Preferred content of the tablet comprising the polymorphic form 6 of sofosbuvir is of at least 40 weight-%. More preferably, the tablet comprises the polymorphic form 6 in an amount in the range of from 40 to 95 weight-%, more preferably from 40 to 90 weight-%, more preferably from 45 to 90 weight-%, more preferably from 45 to 85 weight-%, more preferably from 45 to 80 weight-%, more preferably from 50 to 80 weight-%, based on the total weight of the tablet. More preferably, the tablet comprises the polymorphic form 6 in an amount of from 50 to 75 weight-% based on the total weight of the tablet. Preferred ranges include, for example, a range of from 50 to 60 weight-% or a range of from 55 to 65 weight-% or a range of from 60 to 70 weight-% or a range of from 65 to 75 weight-%. More preferably, the tablet comprises the polymorphic form 6 in an amount of ≧75 weight-% based on the total weight of the tablet. Preferred ranges include, for example, a range of from 75 to 90 weight-% or a range of from 75 to 85 weight-%, or a range of from 75 to 80 weight-% or a range of from 80 to 90 weight-% or a range of from 85 to 90 weight-% based on the total weight of the tablet.

Tablets comprising the polymorphic form 7 of sofosbuvir as defined according to the present invention in an amount in the range of at least 25 weight-%, at least 30 weight-%, at least 35 weight-%, or at least 36 weight-% or at least 37 weight-% or at least 38 weight-% or at least 39 weight-% are embodiments of the present invention. Preferred content of the tablet comprising the polymorphic form 7 of sofosbuvir is at least 40 weight-%. More preferably, the tablet comprises the polymorphic form 7 in an amount in the range of from 40 to 95 weight-%, more preferably from 40 to 90 weight-%, more preferably from 45 to 90 weight-%, more preferably from 45 to 85 weight-%, more preferably from 45 to 80 weight-%, more preferably from 50 to 80 weight-%, based on the total weight of the tablet. More preferably, the tablet comprises the polymorphic form 7 in an amount of from 50 to 75 weight-% based on the total weight of the tablet. Preferred ranges include, for example, a range of from 50 to 60 weight-% or a range of from 55 to 65 weight-% or a range of from 60 to 70 weight-% or a range of from 65 to 75 weight-%. More preferably, the tablet comprises the polymorphic form 7 in an amount of ≧75 weight-% based on the total weight of the tablet. Preferred ranges include, for example, a range of from 75 to 90 weight-% or a range of from 75 to 85 weight-%, or a range of from 75 to 80 weight-% or a range of from 80 to 90 weight-% or a range of from 85 to 90 weight-% based on the total weight of the tablet.

Even more precisely, the moisture stability of the polymorphic form is defined as the amount in mg of the polymorphic form which is present after an exposure of an amount of 30 mg of the polymorphic form to a relative humidity of (75±1) % at a temperature of (40±1)° C. for an exposure time of (18±1) hours in a humidity chamber, relative to the amount of 30 mg of the polymorphic form before said exposure, as determined via X-ray powder diffraction pattern analysis at a temperature in the range of from 15 to 25° C. with Cu-Kalpha_{1,2} radiation having a wavelength of 0.15419 nm of the polymorphic form before the exposure and via said X-ray powder diffraction pattern analysis after the exposure.

Preferably, the polymorphic form has a moisture stability of at least 96%, more preferably at least 97%, more preferably at least 98%, more preferably at least 99%. For example, the polymorphic form has a moisture stability of 100%. For example, a moisture stability of 100% relates to a polymorphic form which, after having been exposed to a relative humidity of (75±1) % at a temperature of (40±1)° C. for an exposure time of (18±1) hours as described above, is not present in another polymorphic form and/or is not present in amorphous form and/or has not deliquesced.

Generally, no specific restrictions exists how a polymorphic form exhibiting the moisture stability as defined above is provided or prepared. Preferably, the polymorphic form is obtainable or obtained by a process comprising crystallization of the polymorphic form in the presence of at least one organic solvent and preferably at least one organic anti-solvent. Generally, the polymorphic form, after having been crystallized and when being used as starting material for the preparation of the tablet, may or may not contain at least traces of the at least one organic solvent. Therefore, the tablet of the present invention may comprise the at least one organic solvent which is contained in the polymorphic form due to its crystallization in this at least one organic solvent. Further, it is also possible that the polymorphic form, after having been crystallized and when being used as starting material for the preparation of the tablet, does not contain any more the at least one organic solvent in which the polymorphic form has been crystallized. In such a case, it is possible that the tablet of the present invention contains the at least one organic solvent in which the polymorphic form has been crystallized, or at least one other suitable organic solvent, wherein this organic solvent is used as starting material for the preparation tablet in addition to the polymorphic form.

Generally, the tablet of the invention comprises the at least one organic solvent in small amounts, preferably in amount of at most 1 weight-%, more preferably at most 0.9 weight-%, more preferably at most 0.8 weight-%, more preferably at most 0.7 weight-%, more preferably at most 0.6 weight-%, more preferably at most 0.5 weight-%, more preferably at most 0.4 weight-%, based on the total weight of the tablet. More preferably, the tablet comprises the at least one organic solvent in an amount in the range of from 0.002 to 0.3 weight-%, more preferably of from 0.005 to 0.2 weight-%, more preferably from 0.01 to 0.1 weight-%, based on the total weight of the tablet. If the at least one organic solvent consists of two or more organic solvents, the amounts mentioned above are to be understood as referring the total amount of all organic solvents.

Preferably, the at least one organic solvent comprises an aliphatic alcohol, more preferably a C₂ alcohol, a C₃ alcohol, a C₄ alcohol, a C₅ alcohol, or a mixture of two or more thereof, more preferably a C₂ alcohol, a C₃ alcohol, a C₄ alcohol, or a mixture of two or more thereof, more preferably ethanol, n-propanol, n-butanol, or a mixture of two or more thereof. More preferably, the at least one organic solvent comprises ethanol or n-butanol. More preferably, the at least one organic solvent is ethanol or n-butanol.

Generally, the polymorphic form exhibiting the moisture stability as defined above can be obtainable or obtained by a process comprising crystallization of the polymorphic form in the presence of at least one organic anti-solvent, in addition to the at least one organic solvent. Generally, the polymorphic form, after having been crystallized and when being used as starting material for the preparation of the tablet, may or may not contain at least traces of the at least one organic anti-solvent. Therefore, the tablet of the present invention may comprise the at least one organic anti-solvent which is contained in the polymorphic form due to its crystallization in this at least one organic anti-solvent. Further, it is also possible that the polymorphic form, after having been crystallized and when being used as starting material for the preparation of the tablet, does not contain any more the at least one organic anti-solvent in which the polymorphic form has been crystallized. In such a case, it is possible that the tablet of the present invention contains the at least one organic anti-solvent in which the polymorphic form has been crystallized, or at least one other suitable organic anti-solvent, wherein this organic anti-solvent is used as starting material for the preparation tablet in addition to the polymorphic form.

Generally, the tablet of the invention comprises the at least one organic anti-solvent in small amounts, preferably in amount of at most 0.5 weight-%, more preferably at most 0.4 weight-%, more preferably at most 0.3 weight-%, more preferably at most 0.2 weight-%, more preferably at most 0.1 weight-%, more preferably at most 0.09 weight-%, more preferably at most 0.08 weight-%, more preferably at most 0.07 weight-%, more preferably at most 0.06 weight-%, based on the total weight of the tablet. More preferably, the tablet comprises the at least one organic anti-solvent in an amount in the range of from 0.0002 to 0.05 weight-%, more preferably of from 0.0005 to 0.03 weight-%, more preferably of from 0.001 to 0.01 weight-%, based on the total weight of the tablet. If the at least one organic anti-solvent consists of two or more organic anti-solvents, the amounts mentioned above are to be understood as referring the total amount of all organic anti-solvents.

Preferably, the at least one organic anti-solvent comprises an alkane, preferably a C₅ alkane, a C₆ alkane, a C₇ alkane, a C₈ alkane, or a mixture of two or more thereof, more preferably a C₇ alkane. More preferably, the at least one organic anti-solvent comprises n-heptane. More preferably, the at least one organic anti-solvent is n-heptane.

According to a conceivable embodiment, the present invention relates to a tablet comprising a polymorphic form of crystalline sofosbuvir according to formula (I)

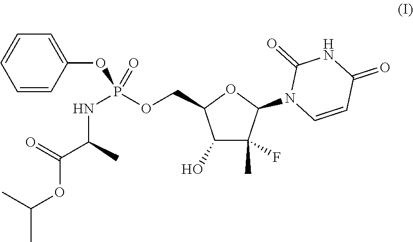 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

in an amount of at least 40 weight-%, preferably from 40 to 90 weight-%, more preferably from 45 to 80 weight-%, more preferably from 50 to 75 weight-% or in an amount of ≧75 weight-% based on the total weight of the tablet or in a range of from 75 to 90 weight-% or in a range of from 75 to 85 weight-%, or in a range of from 75 to 80 weight-% or in a range of from 80 to 90 weight-% or in a range of from 85 to 90 weight-% based on the total weight of the tablet, wherein the polymorphic form is obtainable or obtained by a process comprising crystallization of the polymorphic form in the presence of at least one organic solvent and optionally at least one organic anti-solvent, wherein the tablet may further comprise at least one organic solvent, preferably the at least one organic solvent as described above, preferably in the amounts as described above, wherein the tablet optionally further comprises at least one organic anti-solvent as described above, preferably in the amounts as described above.

According to a conceivable embodiment, the present invention relates to a tablet comprising a polymorphic form of crystalline sofosbuvir according to formula (I)

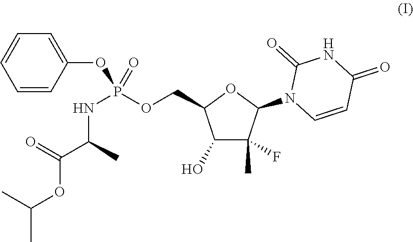 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

in an amount of at least 40 weight-%, preferably from 40 to 90 weight-%, more preferably from 45 to 80 weight-%, more preferably from 50 to 75 weight-% or in an amount of ≧75 weight-% based on the total weight of the tablet or in a range of from 75 to 90 weight-% or in a range of from 75 to 85 weight-%, or in a range of from 75 to 80 weight-% or in a range of from 80 to 90 weight-% or in a range of from 85 to 90 weight-%, based on the total weight of the tablet, wherein the tablet further comprises at least one organic solvent, preferably the at least one organic solvent as described above, preferably in the amounts as described above, wherein the tablet optionally further comprises at least one organic anti-solvent as described above, preferably in the amounts as described above.

### Polymorphic Form 6 of Sofosbuvir

Preferably, the polymorphic form comprised in the tablet is polymorphic form 6 of sofosbuvir having an X-ray powder diffraction pattern with reflections at 2-theta values of (6.1±0.2)°, (8.2±0.2)°, (10.4±0.2)°, (12.7±0.2)°, (17.2±0.2)°, (17.7±0.2)°, (18.0±0.2)°, (18.8±0.2)°, (19.4±0.2)°, (19.8±0.2)°, (20.1±0.2)°, (20.8±0.2)°, (21.8±0.2)°, (23.3±0.2)°, when measured at a temperature in the range of from 15 to 25° C. with Cu-Kalpha_{1,2} radiation having a wavelength of 0.15419 nm. Polymorphic form 6 of sofosbuvir and a process for its preparation are described, for example, in WO 2011/123645 A.

The term “agitation” as used in some of these embodiments and in the respective context of the present invention relates to any motion of a macroscopic constituent of the solution comprising sofosbuvir which is induced from outside, relative to another macroscopic constituent of the solution. The term “mechanical agitation” as used in some of these embodiments and in the respective context of the present invention relates to any motion of a macroscopic constituent of the solution comprising sofosbuvir which is induced from outside via a device, such as shaking or stirring or sonication, relative to another macroscopic constituent of the solution. The term “stirring” as used in some of these embodiments and in the respective context of the present invention to any motion of a macroscopic constituent of the solution comprising sofosbuvir which is induced from outside via a stirring device, relative to another macroscopic constituent of the solution.

Preferably, according to the present invention, the polymorphic form 6 of sofosbuvir and a process for its preparation are characterized by the following embodiments and combinations of embodiments as indicated by the respective dependencies and references:
1. A process for preparing polymorphic form 6 of crystalline sofosbuvir according to formula (I)

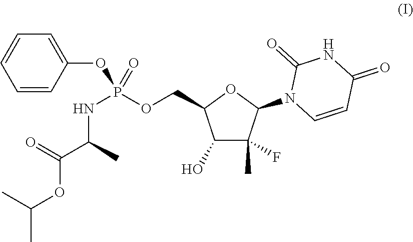 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

having an X-ray powder diffraction pattern with reflections at 2-theta values of (6.1±0.2)°, (8.2±0.2)°, (10.4±0.2)°, (12.7±0.2)°, (17.2±0.2)°, (17.7±0.2)°, (18.0±0.2)°, (18.8±0.2)°, (19.4±0.2)°, (19.8±0.2)°, (20.1±0.2)°, (20.8±0.2)°, (21.8±0.2)°, (23.3±0.2)°, when measured at a temperature in the range of from 15 to 25° C. with Cu-Kalpha_{1,2} radiation having a wavelength of 0.15419 nm, in particular as described in Reference Example 6 herein, the process comprising
   (i) providing sofosbuvir according to formula (I) in crystalline form, pseudo-crystalline form, amorphous form, or as a mixture of two or more of these forms;
   (ii) preparing a mixture comprising the sofosbuvir provided in (i) and at least one organic solvent;
   (iii) subjecting the mixture obtained in (ii) to crystallization conditions, obtaining polymorphic form 6 of sofosbuvir in its mother liquor.
2. The process of embodiment 1, wherein according to (i), the sofosbuvir is provided in polymorphic form 1 having an X-ray powder diffraction pattern with reflections at 2-theta angles of (5.0±0.2)°, (7.3±0.2)°, (9.4±0.2)°, (16.6±0.2)°, (17.3±0.2)°, (18.1±0.2)°, (22.0±0.2)°, (25.0±0.2)°, when measured at a temperature in the range of from 15 to 25° C. with Cu-Kalpha_{1,2} radiation having a wavelength of 0.15419 nm, in particular determined according to Reference Example 6 herein;
   or in polymorphic form 6 having an X-ray powder diffraction pattern with reflections at 2-theta angles of (6.1±0.2)°, (8.2±0.2)°, (10.4±0.2)°, (12.7±0.2)°, (17.2±0.2)°, (17.7±0.2)°, (18.0±0.2)°, (18.8±0.2)°, (19.4±0.2)°, (19.8±0.2)°, (20.1±0.2)°, (20.8±0.2)°, (21.8±0.2)°, when measured at a temperature in the range of from 15 to 25° C. with Cu-Kalpha_{1,2} radiation having a wavelength of 0.15419 nm, in particular determined according to Reference Example 6 herein;
   or in polymorphic form 7 having an X-ray powder diffraction pattern comprising no reflection at 2-theta angles in the range of from 2.0 to 7.8° and preferably comprising reflections at 2-theta angles of (8.1±0.2)°, (10.4±0.2)°, (12.4±0.2)°, (17.3±0.2)°, (19.4±0.2)°, when measured at a temperature in the range of from 15 to 25° C. with Cu-Kalpha_{1,2} radiation having a wavelength of 0.15419 nm, in particular determined according to Reference Example 6 herein;
   or in amorphous form;
   or as a mixture of two, three, or four these forms;
   preferably in polymorphic form 1, in polymorphic form 7, in amorphous form, or as a mixture of two or three of these forms.
3. The process of embodiment 2, wherein according to (i), the sofosbuvir is provided in polymorphic form 7 having an X-ray powder diffraction pattern comprising no reflection at 2-theta angles in the range of from 2.0 to 7.8°, wherein the polymorphic form 7 of sofosbuvir having an X-ray powder diffraction pattern comprising no reflection at 2-theta angles in the range of from 2.0 to 7.8° preferably
   exhibits a Fourier transform infrared spectrum comprising peaks at wavenumbers of (3252±2) cm⁻¹, (2928±2) cm⁻¹, (1718±2) cm⁻¹, (1668±2) cm⁻¹, (1456±2) cm⁻¹, when measured at a temperature in the range of from 15 to 25° C. using a ZnSe ATR cell, in particular determined according to Reference Example 7.1; and/or
   has the monoclinic space group symmetry P2₁ and the following unit cell parameters as determined by an X-ray single-crystal structure analysis at 120 K, in particular as described in Reference Example 7.5 herein:
      a=(5.16±0.04) Angstrom;
      b=(16.86±0.12) Angstrom;
      c=(14.44±0.10) Angstrom;
      alpha=90.0°;
      beta=(100.2±0.8)°;
      gamma=90.0°; and/or
   has a melting point in the range of from 122 to 126° C. when measured via differential scanning calorimetry at a heating rate of 10° C./min at a pressure in the range of from 0.95 to 1.05 bar, in particular determined according to Reference Example 7.2 herein; and/or
   comprises at most 0.5 weight-% of organic solvent, based on the weight of the polymorphic form 7, as determined via thermogravimetric analysis, in particular determined according to Reference Example 7.3; and/or
   comprising at most 0.4 weight-% of water based on the weight of the polymorphic form 7 as determined via gravimetric moisture sorption/desorption analysis at a temperature of (25.0±0.1)° C. and a relative humidity of from 0 to 95%, in particular determined according to Reference Example 7.4 herein.
4. The process of embodiment 3, wherein the polymorphic form 7 of sofosbuvir is prepared by a process comprising, or wherein the process of embodiment 2 or 3 comprises preparing the polymorphic form 7 by a process comprising
   (a1) providing sofosbuvir in crystalline form, pseudo-crystalline form, amorphous form, or as a mixture of two or more of these forms, wherein the sofosbuvir is preferably provided in polymorphic form 1 having an X-ray powder diffraction pattern with reflections at 2-theta angles of (5.0±0.2)°, (7.3±0.2)°, (9.4±0.2)°, (16.6±0.2)°, (17.3±0.2)°, (18.1±0.2)°, (22.0±0.2)°, (25.0±0.2)°, when measured at a temperature in the range of from 15 to 25° C. with Cu-Kalpha_{1,2} radiation having a wavelength of 0.15419 nm; or in polymorphic form 6 having an X-ray powder diffraction pattern with reflections at 2-theta angles of (6.1±0.2)°, (8.2±0.2)°, (10.4±0.2)°, (12.7±0.2)°, (17.2±0.2)°, (17.7±0.2)°, (18.0±0.2)°, (18.8±0.2)°, (19.4±0.2)°, (19.8±0.2)°, (20.1±0.2)°, (20.8±0.2)°, (21.8±0.2)°, when measured at a temperature in the range of from 15 to 25° C. with Cu-Kalpha_{1,2} radiation having a wavelength of 0.15419 nm; or in polymorphic form 7 having an X-ray powder diffraction pattern comprising no reflection at 2-theta angles in the range of from 2.0 to 7.8° and preferably comprising reflections at 2-theta angles of (8.1±0.2)°, (10.4±0.2)°, (12.4±0.2)°, (17.3±0.2)°, (19.4±0.2)°, when measured at a temperature in the range of from 15 to 25° C. with Cu-Kalpha_{1,2} radiation having a wavelength of 0.15419 nm; or in amorphous form; or as a mixture of two, three, or four of these forms, more preferably in polymorphic form 1, in polymorphic form 6, in amorphous form, or as a mixture of two or three of these forms, more preferably in polymorphic form 1;
   (b1) preparing seed crystals of the polymorphic form 7 of sofosbuvir by a method comprising
      (b1.1) providing sofosbuvir in crystalline form, pseudo-crystalline form, amorphous form, or as a mixture of two or more of these forms, preferably in polymorphic form 1 having an X-ray powder diffraction pattern with reflections at 2-theta values of (5.0±0.2)°, (7.3±0.2)°, (9.4±0.2)°, (16.6±0.2)°, (17.3±0.2)°, (18.1±0.2)°, (22.0±0.2)°, (25.0±0.2)°, when measured at a temperature from 15 to 25° C. with Cu-Kalpha_{1,2} radiation having a wavelength of 0.15419 nm;
      (b1.2) providing seed crystals of polymorphic form 6 of sofosbuvir, having an X-ray powder diffraction pattern with reflections at 2-theta values of (6.1±0.2)°, (8.2±0.2)°, (10.4±0.2)°, (12.7±0.2)°, (20.8±0.2)°, when measured at a temperature in the range of from 15 to 25° C. with Cu-Kalpha_{1,2} radiation having a wavelength of 0.15419 nm;
      (b1.3) preparing a solution of the sofosbuvir provided in (b1.1) in a C₂-C₁₀ alcohol or in a mixture of two or more thereof, preferably in a C₅-C₁₀ alcohol or in a mixture of two or more thereof, more preferably in a C₈ alcohol, more preferably in 2-octanol;
      (b1.4) subjecting the solution provided in (b1.3) to crystallization conditions, comprising seeding the solution with the seed crystals provided in (b1.2), wherein during crystallization, the solution is not stirred, preferably not mechanically agitated, more preferably not agitated, obtaining the polymorphic form 7 of sofosbuvir in its mother liquor, wherein during crystallization in (b1.4), the solution is kept at a temperature in the range of from 15 to 35° C., preferably from 20 to 30° C., at a pressure in the range of from 0.95 to 1.05 bar;
      (b1.5) separating at least a portion of the polymorphic form 7 from its mother liquor, preferably comprising
         (b1.5.1) subjecting the mother liquor comprising the polymorphic form 7 of sofosbuvir to a solids separation process, preferably to filtration, obtaining the polymorphic form 7 of sofosbuvir;
         (b1.5.2) optionally washing the polymorphic form 7 of sofosbuvir obtained in (b1.5.1);
         (b1.5.3) preferably drying the polymorphic form 7 of sofosbuvir obtained in (b1.5.1) or (b1.5.2);
   (c1) preparing a solution of sofosbuvir provided in (a1) in a C₂-C₅ alcohol or in a mixture of two or more thereof, and in one or more anti-solvents, preferably comprising
      (c1.1) preparing a solution of the sofosbuvir provided in (a1) in the C₂-C₅ alcohol or in the mixture of two or more thereof, preferably in ethanol, or n-butanol, or n-pentanol, wherein preparing the solution preferably comprises heating, preferably heating the solution to a temperature in the range of from 30 to 70° C., preferably from 35 to 65° C.;
      (c2.2) adding the one or more anti-solvents to the solution obtained in (c1.1), wherein the one or more anti-solvents preferably comprise an alkane, more preferably a C₅ alkane, a C₆ alkane, a C₇ alkane, a C₈ alkane, or a mixture of two or more thereof, more preferably a C₇ alkane, more preferably n-heptane;
      wherein after (c1.1) and before (c1.2), the solution is preferably cooled, preferably to a temperature in the range of from 15 to 35° C., more preferably from 20 to 30° C.; and wherein after (c1.1) and before (c1.2), the solution obtained in (c1.1) is optionally subjected to a solids separation process, preferably filtration;
   (d1) subjecting the solution provided in (c1) to crystallization conditions, comprising seeding the solution with the seed crystals prepared in (b1), wherein
      wherein during crystallization, the solution is not stirred, preferably not mechanically agitated, more preferably not agitated;
      or wherein during crystallization, at least a portion of the seeded solution, preferably at least the portion of the seeded solution containing most of the seed crystals, more preferably at least the portion of the seeded solution containing most of the seed crystals and the polymorphic form 7 formed, in particular at least the bottom of the seeded solution containing most of the seed crystals, preferably essentially all seed crystals, and the polymorphic form 7 formed;
   obtaining the crystalline form 7 of sofosbuvir in its mother liquor, wherein during crystallization, the solution is kept at a temperature in the range of from 15 to 35° C., preferably from 20 to 30° C., at a pressure in the range of from 0.95 to 1.05 bar;
   (e1) preferably separating the polymorphic form 7 of sofosbuvir from its mother liquor, preferably comprising
      (e1.1) subjecting the mother liquor comprising the polymorphic form 7 of sofosbuvir to filtration, obtaining at least a portion of the polymorphic form 7 of sofosbuvir;
      (e1.2) preferably washing the polymorphic form 7 of sofosbuvir obtained in (e1.1);
      (e1.3) preferably drying the polymorphic form 7 of sofosbuvir obtained in (e1.1) or (e1.2).
5. The process of embodiment 2, wherein according to (i), the sofosbuvir is provided in polymorphic form 1.
6. The process of embodiment 2, wherein according to (i), the sofosbuvir is provided in amorphous form.
7. The process of any of embodiments 1 to 6, wherein (ii) comprises (ii.1) preparing a solution of the sofosbuvir provided in (i) in at least one organic solvent.
8. The process of embodiment 7, wherein the at least one organic solvent in (ii) comprises an aliphatic alcohol, preferably a C₂ alcohol, a C₃ alcohol, a C₄ alcohol, a C₅ alcohol, or a mixture of two or more thereof, more preferably a C₂ alcohol, a C₃ alcohol, a C₄ alcohol, or a mixture of two or more thereof, more preferably ethanol, n-propanol, n-butanol, or a mixture of two or more thereof
9. The process of embodiment 8, wherein the at least one organic solvent in (ii) comprises, preferably is, n-butanol.
10. The process of embodiment 8, wherein the at least one organic solvent in (ii) comprises, preferably is, ethanol.
11. The process of any embodiments 7 to 10, wherein preparing the solution in (ii.1) comprises mixing the sofosbuvir provided in (i) with the at least one organic solvent at a temperature in the range of from 20 to 80° C., preferably in the range of from 20 to 70° C., more preferably in the range of from 25 to 70° C.
12. The process of any of embodiments 7 to 11, wherein preparing the solution in (ii.1) comprises agitating, preferably stirring, subjecting to sonication, or a combination thereof, wherein (ii.1) optionally comprises subjecting the solution to a solids separation process, preferably comprising filtration or centrifugation.
13. The process of any of embodiments 7 to 12, further comprising (ii.2) adding at least one organic anti-solvent to the solution obtained in (ii.1).
14. The process of embodiment 13, wherein the at least one organic anti-solvent comprises an alkane, preferably a C₅ alkane, a C₆ alkane, a C₇ alkane, a C₈ alkane, or a mixture of two or more thereof, more preferably a C₇ alkane.
15. The process of embodiment 14, wherein the at least one organic anti-solvent comprises, preferably is, n-heptane.
16. The process of any of embodiments 13 to 15, wherein in (ii.2), adding the at least one organic anti-solvent to the solution obtained in (ii.1) is carried out a temperature in the range of from 20 to 80° C., preferably in the range of from 20 to 70° C., more preferably in the range of from 25 to 70° C.
17. The process of any of embodiments 13 to 16, wherein in (ii.2), adding the at least one organic anti-solvent comprises stirring, subjecting to sonication, or a combination thereof.
18. The process of any of embodiments 1 to 6, wherein preparing the mixture in (ii) comprises suspending the sofosbuvir provided in (i) in a mixture of the at least one organic solvent and at least one organic anti-solvent.
19. The process of embodiment 18, wherein the at least one organic solvent comprises an aliphatic alcohol, preferably a C₂ alcohol, a C₃ alcohol, a C₄ alcohol, a C₅ alcohol, or a mixture of two or more thereof, more preferably a C₂ alcohol, a C₃ alcohol, a C₄ alcohol, or a mixture of two or more thereof, more preferably ethanol, n-propanol, n-butanol, or a mixture of two or more thereof
20. The process of embodiment 19, wherein the at least one organic solvent in (ii) comprises, preferably is, n-butanol.
21. The process of embodiment 19, wherein the at least one organic solvent in (ii) comprises, preferably is, ethanol.
22. The process of any of embodiments 18 to 21, wherein the at least one organic anti-solvent comprises an alkane, preferably a C₅ alkane, a C₆ alkane, a C₇ alkane, a C₈ alkane, or a mixture of two or more thereof, more preferably a C₇ alkane.
23. The process of embodiment 22, wherein the at least one organic anti-solvent comprises, preferably is, n-heptane.
24. The process of any of embodiments 18 to 23, wherein preparing the mixture in (ii) comprises suspending the sofosbuvir provided in (i) in the mixture of the at least one organic solvent and the at least one organic anti-solvent at a temperature in the range of from 10 to 30° C., preferably in the range of from 15 to 25° C.
25. The process of any of embodiments 13 to 24, wherein the volume ratio of the at least one organic solvent relative to the at least one anti-solvent is in the range of from 0.2:1 to 1:1, preferably in the range of from 0.4:1 to 0.9:1.
26. The process of any of embodiments 11 to 21, wherein the mixture prepared in (ii) contains the sofosbuvir, relative to the at least one organic solvent, in an amount in the range of from 100 to 500 mg/mL, preferably in the range of from 200 to 475 mg/mL, more preferably in the range of from 250 to 450 mg/mL.
27. The process of any of embodiments 1 to 26, wherein in (iii), the crystallization conditions comprise a crystallization temperature in the range of from 0 to 30° C.
28. The process of any of embodiments 1 to 27, wherein in (iii), the crystallization conditions comprise a crystallization time in the range of from 0.1 to 16 h, preferably in the range of from 0.5 to 9 h, more preferably in the range of from 1 to 6 h.
29. The process of any of embodiments 1 to 29, wherein in (iii), the crystallization conditions comprise adding at least one anti-solvent to the mixture being subjected to crystallization conditions, wherein the at least one organic anti-solvent preferably comprises an alkane, more preferably a C₅ alkane, a C₆ alkane, a C₇ alkane, a C₈ alkane, or a mixture of two or more thereof, more preferably a C₇ alkane, more preferably n-heptane.
30. The process of any of embodiments 1 to 29, further comprising
(iv) separating the polymorphic form 6 of sofosbuvir from its mother liquor.
31. The process of embodiment 30, wherein separating in (iv) comprises (iv.1) subjecting the mother liquor containing the polymorphic form 6 of sofosbuvir to a solids separation process, obtaining the polymorphic form 6.
32. The process of embodiment 31, wherein in (iv.1), the solids separation process comprises filtration, centrifugation, or a combination of thereof, preferably filtration.
33. The process of embodiment 31 or 32, wherein separating in (iv) further comprises (iv.2) washing the polymorphic form 6 of sofosbuvir obtained from (iv.1).
34. The process of embodiment 33, wherein in (iv.2), the washing of the polymorphic form 6 of sofosbuvir is carried out with at least one anti-solvent, wherein the at least one organic anti-solvent preferably comprises an alkane, more preferably a C₅ alkane, a C₆ alkane, a C₇ alkane, a C₈ alkane, or a mixture of two or more thereof, more preferably a C₇ alkane, more preferably n-heptane.
35. The process of embodiment 33 or 34, wherein in (iv.2), the washing is carried out at a temperature in the range of from 0 to 30° C.
36. The process of any of embodiments 30 to 35, wherein separating in (iv) further comprises (iv.3) drying the polymorphic form 6 of sofosbuvir obtained from (iv.1), optionally from (iv.2).
37. The process of embodiment 36, wherein in (iv.3), the drying is carried out at a temperature in the range of from 10 to 60° C., preferably in the range of from 15 to 55° C., more preferably in the range of from 20 to 50° C.
38. The process of embodiment 36 or 37, wherein in (iv.3), the drying is carried out at a pressure in the range of from 10 to 250 mbar, preferably in the range of from 10 to 100 mbar, more preferably in the range of from 10 to 50 mbar.
39. Polymorphic form 6 of sofosbuvir, obtainable of obtained by a process according to any of embodiments 1 to 38.

### Polymorphic Form 7 of Sofosbuvir

Also preferably, the polymorphic form comprised in the tablet is polymorphic form 7 of sofosbuvir having an X-ray powder diffraction pattern comprising no reflection at 2-theta angles in the range of from 2.0 to 7.8°, preferably comprising reflections at 2-theta values of (8.1±0.2)°, (10.4±0.2)°, (12.4±0.2)°, (17.3±0.2)°, (19.4±0.2)°, when measured at a temperature in the range of from 15 to 25° C. with Cu-Kalpha_{1,2} radiation having a wavelength of 0.15419 nm. The polymorphic form 7 of sofosbuvir and a process for its preparation are characterized by the following embodiments and combinations of embodiments as indicated by the respective dependencies and references:
1. Polymorphic form 7 of crystalline sofosbuvir of formula (I)

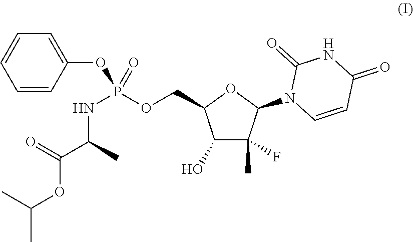 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

having an X-ray powder diffraction pattern comprising no reflection at 2-theta angles in the range of from 2.0 to 7.8°, when measured at a temperature in the range of from 15 to 25° C. with Cu-Kalpha_{1,2} radiation having a wavelength of 0.15419 nm, in particular as described in Reference Example 6 herein.
2. The polymorphic form of embodiment 1, having an X-ray powder diffraction pattern comprising reflections at 2-theta angles of (8.1±0.2)°, (10.4±0.2)°, (12.4±0.2)°, (17.3±0.2)°, (19.4±0.2)°, when measured at a temperature in the range of from 15 to 25° C. with Cu-Kalpha_{1,2} radiation having a wavelength of 0.15419 nm, preferably comprising additional reflections at 2-theta angles of (12.1±0.2)°, (13.5±0.2)°, (16.2±0.2)°, (16.8±0.2)°, (18.0±0.2)°, (18.7±0.2)°, (20.2±0.2)°, (20.9±0.2)°, (22.1±0.2)°, (23.4±0.2)°, (25.4±0.29°, (28.0±0.2)°, when measured at a temperature in the range of from 15 to 25° C. with Cu-Kalpha_{1,2} radiation having a wavelength of 0.15419 nm, in particular as described in Reference Example 6 herein, more preferably comprising reflections at 2-theta angles according to the following peak list:

| position/ | |
|---|---|
| ° 2-theta | relative intensity/% |
| 8.1 ± 0.2 | 75 |
| 10.4 ± 0.2 | 46 |
| 12.1 ± 0.2 | 9 |
| 12.4 ± 0.2 | 78 |
| 13.5 ± 0.2 | 14 |
| 16.2 ± 0.2 | 10 |
| 16.8 ± 0.2 | 30 |
| 17.3 ± 0.2 | 100 |
| 18.0 ± 0.2 | 21 |
| 18.7 ± 0.2 | 14 |
| 19.4 ± 0.2 | 68 |
| 20.1 ± 0.2 | 49 |
| 20.9 ± 0.2 | 48 |
| 22.1 ± 0.2 | 13 |
| 23.1 ± 0.2 | 5 |
| 23.4 ± 0.2 | 24 |
| 23.7 ± 0.2 | 15 |
| 25.0 ± 0.2 | 18 |
| 25.4 ± 0.2 | 20 |
| 27.2 ± 0.2 | 25 |
| 28.0 ± 0.2 | 15 |
| 28.3 ± 0.2 | 6.8 |
| 28.6 ± 0.2 | 9 |
| 31.3 ± 0.2 | 9.4 |

3. The polymorphic form of embodiment 1 or 2, exhibiting a Fourier transform infrared spectrum comprising peaks at wavenumbers of (3252±2) cm⁻¹, (2928±2) cm⁻¹, (1718±2) cm⁻¹, (1668±2) cm⁻¹, (1456±2) cm⁻¹, when measured at a temperature in the range of from 15 to 25° C. using a ZnSe ATR cell, preferably comprising additional peaks at wavenumbers of (1494±2) cm⁻¹, (1373±2) cm⁻¹, (1265±2) cm⁻¹, (1223±2) cm⁻¹, (945±2) cm⁻¹, when measured at a temperature in the range of from 15 to 25° C. using a ZnSe ATR cell, in particular as described in Reference Example 7.1 herein.
4. The polymorphic form of any of embodiments 1 to 3, the monoclinic space group symmetry P2₁ and the following unit cell parameters as determined by an X-ray single-crystal structure analysis at 120 K, in particular as described in Reference Example 7.5 herein:
   a=(5.16±0.04) Angstrom;
   b=(16.86±0.12) Angstrom;
   c=(14.44±0.10) Angstrom;
   alpha=90.0°;
   beta=(100.2±0.8)°;
   gamma=90.0°.
5. The polymorphic form of any of embodiments 1 to 4, having a melting point in the range of from 122 to 126° C. when measured via differential scanning calorimetry at a heating rate of 10 K/min at a pressure in the range of from 0.95 to 1.05 bar, in particular as described in Reference Example 7.2 herein.
6. The polymorphic form of any of embodiments 1 to 5, comprising at most 0.5 weight-% of organic solvent, based on the weight of the polymorphic form, as determined via thermogravimetric analysis, in particular as described in Reference Example 7.3 herein.
7. The polymorphic form of any of embodiments 1 to 6, comprising at most 0.4 weight-% of water based on the weight of the polymorphic form as determined via gravimetric moisture sorption/desorption analysis at a temperature of (25.0±0.1)° C. and a relative humidity of from 0 to 95%, in particular as described in Reference Example 7.4 herein.
8. A process for the preparation of the polymorphic form 7 of crystalline sofosbuvir of formula (I) according to any of embodiments 1 to 7, comprising
   (i) providing sofosbuvir in crystalline form, pseudo-crystalline form, amorphous form, or as a mixture of two or more of these forms;
   (ii) preparing seed crystals comprising the polymorphic form 7 of sofosbuvir according to any of embodiments 1 to 7 by a method comprising
      (ii.1) providing sofosbuvir in crystalline form, pseudo-crystalline form, amorphous form, or as a mixture of two or more of these forms;
      (ii.2) providing seed crystals of polymorphic form 6 of crystalline sofosbuvir, having an X-ray powder diffraction pattern with reflections at 2-theta values of (6.1±0.2)°, (8.2±0.2)°, (10.4±0.2)°, (12.7±0.2)°, (20.8±0.2) °, when measured at a temperature in the range of from 15 to 25° C. with Cu-Kalpha_{1,2} radiation having a wavelength of 0.15419 nm;
      (ii.3) preparing a solution of the sofosbuvir provided in (ii.1) in a C₂-C₁₀ alcohol or in a mixture of two or more thereof;
      (ii.4) subjecting the solution provided in (ii.3) to crystallization conditions, comprising seeding the solution with the seed crystals provided in (ii.2), wherein during crystallization, the solution is not stirred;
      (ii.5) separating at least a portion of the crystalline form of sofosbuvir according to any of embodiments 1 to 7 from its mother liquor;
   (iii) preparing a solution of sofosbuvir provided in (i) in a C₂-C₅ alcohol or in a mixture of two or more thereof, and in one or more anti-solvents;
   (iv) subjecting the solution provided in (iii) to crystallization conditions, comprising seeding the solution with the seed crystals prepared in (ii),
      wherein during crystallization, the solution is not stirred:
      or wherein during crystallization, at least a portion of the seeded solution, preferably at least the portion of the seeded solution containing most of the seed crystals, more preferably at least the portion of the seeded solution containing most of the seed crystals and the polymorphic form 7 formed, in particular at least the bottom of the seeded solution containing most of the seed crystals, preferably essentially all seed crystals, and the polymorphic form 7 formed;
   obtaining the polymorphic form 7 of sofosbuvir according to any of embodiments 1 to 7, in its mother liquor.
9. The process of embodiment 8, wherein in (i) and (ii.1), sofosbuvir is provided in polymorphic form 1 having an X-ray powder diffraction pattern with reflections at 2-theta values of (5.0±0.2)°, (7.3±0.2)°, (9.4±0.2)°, (16.6±0.2)°, (17.3±0.2)°, (18.1±0.2)°, (22.0±0.2)°, when measured at a temperature from 15 to 25° C. with Cu-Kalpha_{1,2} radiation having a wavelength of 0.15419 nm.
10. The process of embodiment 8 or 9, wherein in (ii.3), the solution of the sofosbuvir provided in (ii.1) is prepared in a C₅-C₁₀ alcohol or in a mixture of two or more thereof, more preferably in a C₈ alcohol.
11. The process of any of embodiments 8 to 10, wherein in (ii.3), the solution of the sofosbuvir provided in (ii.1) is prepared in 2-octanol.
12. The process of any of embodiments 8 to 11, wherein in (ii.4), during crystallization, the solution is not mechanically agitated.
13. The process of any of embodiments 8 to 12, wherein in (ii.4), during crystallization, the solution is not agitated.
14. The process of any of embodiments 8 to 13, wherein in (iv), during crystallization, the solution is not mechanically agitated.
15. The process of any of embodiments 8 to 14, wherein in (iv), during crystallization, the solution is not agitated.
16. The process of any of embodiments 8 to 15, wherein during crystallization in (ii.4), the solution is kept at a temperature in the range of from 15 to 35° C., at a pressure in the range of from 0.95 to 1.05 bar.
17. The process of any of embodiments 8 to 16, wherein during crystallization in (ii.4), the solution is kept at a temperature in the range of from 20 to 30° C. at a pressure in the range of from 0.95 to 1.05 bar.
18. The process of any of embodiments 8 to 17, wherein separating in (ii.5) comprises
   (ii.5.1) subjecting the mother liquor comprising the polymorphic form 7 of sofosbuvir according to any of embodiments 1 to 7 to a solids separation process, preferably to filtration, obtaining the polymorphic form 7 of sofosbuvir according to any of embodiments 1 to 7.
19. The process of embodiment 18, wherein separating in (ii.5) further comprises
   (ii.5.2) washing the polymorphic form 7 of sofosbuvir according to any of embodiments 1 to 7 obtained in (ii.5.1).
20. The process of embodiment 18 or 19, wherein separating in (ii.5) further comprises
   (ii.5.3) drying the polymorphic form 7 of sofosbuvir according to any of embodiments 1 to 7 obtained in (ii.5.1) or (ii.5.2).
21. The process of any of embodiments 8 to 20, wherein (iii) comprises
   (iii.1) preparing a solution of the sofosbuvir provided in (i) the C₂-C₅ alcohol or in the mixture of two or more thereof;
   (iii.2) adding the one or more anti-solvents to the solution obtained in (iii.1).
22. The process of embodiment 21, wherein in (iii.1), the preparation of the solution comprises heating.
23. The process of embodiment 22, wherein in (iii.1), the preparation of the solution comprises heating to a temperature in the range of from 30 to 70° C.
24. The process of embodiment 21 or 22, wherein in (iii.1), the preparation of the solution comprises heating to a temperature in the range of from 35 to 65° C.
25. The process of any of embodiments 21 to 24, wherein after (iii.1) and before (iii.2), the solution is cooled.
26. The process of embodiment 25, wherein after (iii.1) and before (iii.2), the solution is cooled to a temperature in the range of from 15 to 35° C.
27. The process of embodiment 25, wherein after (iii.1) and before (iii.2), the solution is cooled to a temperature in the range of from 20 to 30° C.
28. The process of any of embodiments 21 to 27, wherein after (iii.1) and before (iii.2), the solution obtained in (iii.1) is subjected to a solids separation process.
29. The process of embodiment 28, wherein after (iii.1) and before (iii.2), the solution obtained in (iii.1) is subjected to filtration.
30. The process of any of embodiments 8 to 29, wherein in (iii), the solution is prepared in ethanol and in one or more anti-solvents.
31. The process of embodiment 30, wherein in (iii), the molar ratio of ethanol relative to the one or more anti-solvents is in the range of from 0.1:1 to 1:1.
32. The process of embodiment 31, wherein in (iii), the molar ratio of ethanol relative to the one or more anti-solvents is in the range of from 0.3:1 to 0.7:1.
33. The process of any of embodiments 8 to 29, wherein in (iii), the solution is prepared in one or more C₄ alcohols and in one or more anti-solvents.
34. The process of embodiment 33, wherein in (iii), the solution is prepared in n-butanol and in one or more anti-solvents.
35. The process of embodiment 33 or 34, wherein in (iii), the molar ratio of the one or more C₄ alcohols relative to the one or more anti-solvents is in the range of from 0.1:1 to 1:1.
36. The process of embodiment 35, wherein in (iii), the molar ratio of the one or more C₄ alcohols relative to the one or more anti-solvents is in the range of from 0.3:1 to 0.7:1.
37. The process of any of embodiments 8 to 29, wherein in (iii), the solution is prepared in one or more C₅ alcohols and in one or more anti-solvents.
38. The process of embodiment 37, wherein in (iii), the solution is prepared in n-pentanol and in one or more anti-solvents.
39. The process of embodiment 37 or 38, wherein in (iii), the molar ratio of the one or more C₅ alcohols relative to the one or more anti-solvents is in the range of from 0.1:1 to 1:1.
40. The process of embodiment 39, wherein in (iii), the molar ratio of the one or more C₅ alcohols relative to the one or more anti-solvents is in the range of from 0.3:1 to 0.7:1.
41. The process of any of embodiments 8 to 40, wherein in (iii), the one or more anti-solvents comprises an alkane.
42. The process of any of embodiments 8 to 41, wherein in (iii), the one or more anti-solvents comprises a C₅ alkane, a C₆ alkane, a C₇ alkane, a C₈ alkane, or a mixture of two or more thereof.
43. The process of any of embodiments 8 to 42, wherein in (iii), the one or more anti-solvents comprises a C₇ alkane.
44. The process of any of embodiments 8 to 43, wherein in (iii), the one or more anti-solvents comprises n-heptane.
45. The process of any of embodiments 8 to 44, wherein in (iii), the one or more anti-solvents is n-heptane.
46. The process of any of embodiments 8 to 45, wherein in (iv), the solution is seeded with the seed crystals prepared in (ii) in an amount in the range of from 1 to 20 weight-%, based on the weight of sofosbuvir provided in (i).
47. The process of any of embodiments 8 to 46, wherein in (iv), the solution is seeded with the seed crystals prepared in (ii) in an amount in the range of from 1 to 5 weight-%, based on the weight of sofosbuvir provided in (i).
48. The process of any of embodiments 8 to 47, wherein during crystallization in (iv), the solution is kept at a temperature in the range of from 15 to 35° C., at a pressure in the range of from 0.95 to 1.05 bar.
49. The process of any of embodiments 8 to 48, wherein during crystallization in (iv), the solution is kept at a temperature in the range of from 20 to 30° C., at a pressure in the range of from 0.95 to 1.05 bar.
50. The process of any of embodiments 8 to 49, further comprising
   (v) separating the polymorphic form 7 of sofosbuvir according to any of embodiments 1 to 7 from its mother liquor.
51. The process of embodiment 50, wherein separating in (v) comprises (v.1) subjecting the mother liquor comprising the polymorphic form 7 of sofosbuvir according to any of embodiments 1 to 7 to a solids separation process, obtaining at least a portion of the polymorphic form 7 of sofosbuvir according to any of embodiments 1 to 7.
52. The process of embodiment 50 or 51, wherein separating in (v) comprises (v.1) subjecting the mother liquor comprising the polymorphic form 7 of sofosbuvir according to any of embodiments 1 to 7 to filtration, obtaining at least a portion of the polymorphic form 7 of sofosbuvir according to any of embodiments 1 to 7.
53. The process of embodiments 51 or 52, wherein separating in (v) further comprises (v.3) drying the polymorphic form 7 of sofosbuvir according to any of embodiments 1 to 7 obtained in (v.1).
54. The process of embodiment 51 or 52, wherein separating in (v) further comprises
   (v.2) washing the polymorphic form 7 of sofosbuvir according to any of embodiments 1 to 7 obtained in (v.1).
55. The process of embodiment 54, wherein in (v.2), the washing is carried out using one or more anti-solvents as washing agent.
56. The process of embodiment 55, wherein the one or more anti-solvents comprises a C₅ alkane, a C₆ alkane, a C₇ alkane, a C₈ alkane, or a mixture of two or more thereof
57. The process of embodiment 56, wherein the one or more anti-solvents comprises a C₇ alkane.
58. The process of embodiment 57, wherein the one or more anti-solvents comprise n-heptane.
59. The process of embodiment 58, wherein the one or more anti-solvents is n-heptane.
60. The process of any of embodiments 54 to 59, wherein separating in (v) further comprises
   (v.3) drying the polymorphic form 7 of sofosbuvir according to any of embodiments 1 to 7 obtained in (v.2).
61. The process of embodiment 53 or 60, wherein in (v.3), drying is carried out at a temperature in the range of from 10 to 40° C.
62. The process of embodiment 61, wherein in (v.3), drying is carried out at a temperature in the range of from 25 to 35° C.
63. Polymorphic form 7 of sofosbuvir, obtainable of obtained by a process according to any of embodiments 1 to 62.

According to the present invention, it is also possible that the tablet comprises a mixture of polymorphic form 6 and polymorphic form 7 of sofosbuvir. According to a preferred embodiment of the present invention, the tablet comprises polymorphic form 7 of sofosbuvir, wherein more preferably, the polymorphic form of sofosbuvir which is comprised in the tablet is polymorphic form 7 of sofosbuvir. Preferably, the polymorphic form of sofosbuvir comprised in the tablet of the present invention is not polymorphic form 1 and is not polymorphic form 2 and is not polymorphic form 3 and is not polymorphic form 4 and is not polymorphic form 5.

### Pharmaceutically Acceptable Excipients

In addition to the polymorphic form of sofosbuvir, and optionally in addition to the at least one organic solvent as described above and further optionally in addition to the at least one organic anti-solvent as described above, the tablet preferably comprises at least one pharmaceutically acceptable excipient.

The term “pharmaceutically acceptable excipient” as used in this context of the present invention relates to a compound that is used to prepare the tablet, and is generally safe, non-toxic and neither biologically nor otherwise undesirable, and includes excipients that are acceptable for human pharmaceutical use.

With regard to the at least one excipient, no specific restrictions exist provided that the tablet of the invention having the desired dissolution properties are obtained. Conceivable excipients include diluents, disintegrants, glidants, lubricants, coloring agents, taste-masking agents, coating agents, and the like,

Preferably, the at least one pharmaceutically acceptable excipient comprises at least one diluent, or at least one disintegrant, or at least one glidant, or at least one lubricant, or a combination of at least one diluent and at least one disintegrant, or a combination of at least one diluent and at least one glidant, or a combination of at least one disintegrant and at least one lubricant, or a combination of at least one diluent and at least one disintegrant and at least one glidant, or a combination of at least one diluent and at least one disintegrant and at least one lubricant, or a combination of at least one disintegrant and at least one glidant and at least one lubricant, or a combination of at least one diluent and at least one disintegrant and at least one glidant and at least one lubricant. More preferably, the at least one pharmaceutically acceptable excipient comprises a combination of at least one diluent and at least one disintegrant and at least one glidant and at least one lubricant.

More preferably, the at least one pharmaceutically acceptable excipient is a combination of at least one diluent and at least one disintegrant and at least one glidant and at least one lubricant. More preferably, the at least one pharmaceutically acceptable excipient is a combination of at least one diluent and one disintegrant and one glidant and one lubricant.

### Diluent

With regard to the at least one diluent, no specific restrictions exist provided that the tablet of the invention having the desired dissolution properties are obtained.

Preferably, the at least one diluent comprises, preferably is, at least one of calcium carbonate, dicalcium phosphate, dry starch, calcium sulfate, cellulose, compressible sugars, confectioner's sugar, dextrates, dextrin, dextrose, dibasic calcium phosphate dihydrate, glyceryl palmitostearate, hydrogenated vegetable oil type I, inositol, kaolin, lactose, magnesium carbonate, magnesium oxide, maltodextrin, mannitol, microcrystalline cellulose, polymethacrylates, potassium chloride, powdered cellulose, powdered sugar, pregelatinized starch, sodium chloride, sorbitol, starch, sucrose, sugar spheres, talc, tribasic calcium phosphate, more preferably at least one of dicalcium phosphate, cellulose, compressible sugars, dibasic calcium phosphate dehydrate, lactose, mannitol, microcrystalline cellulose, starch, tribasic calcium phosphate, more preferably at least one of mannitol, microcrystalline cellulose.

More preferably, the at least one diluent comprises a combination of mannitol and microcrystalline cellulose. More preferably, the at least one diluent is a combination of mannitol and microcrystalline cellulose.

### Disintegrant

With regard to the at least one disintegrant, no specific restrictions exist provided that the tablet of the invention having the desired dissolution properties are obtained.

Preferably, the at least disintegrant comprises, preferably is, at least one of agar, alginic acid, bentonite, carboxymethylcellulose calcium, carboxymethylcellulose sodium, carboxymethylcellulose, cellulose, a cation exchange resin, cellulose, gums, citrus pulp, colloidal silicon dioxide, corn starch, croscarmellose sodium, crospovidone, guar gum, hydrous aluminum silicate, an ion exchange resin (e.g., polyacrin potassium), magnesium aluminum silicate, methyl cellulose, microcrystalline cellulose, modified cellulose gum, modified corn starch, montmorillonite clay, natural sponge, polyacrilin potassium, potato starch, powdered cellulose, povidone, pregelatinized starch, sodium alginate, sodium bicarbonate in admixture with an acidulant such as tartaric acid or citric acid, sodium starch glycolate, starch, silicates, more preferably at least one of croscarmellose sodium, crospovidone, microcrystalline cellulose, modified corn starch, povidone, pregelatinized starch, sodium starch glycolate.

More preferably, the at least one disintegrant comprises croscarmellose sodium. More preferably, the at least one disintegrant is croscarmellose sodium.

### Glidant

With regard to the at least one glidant, no specific restrictions exist provided that the tablet of the invention having the desired dissolution properties are obtained.

Preferably, the at least one glidant comprises, preferably is, is at least one of colloidal silicon dioxide, talc, starch, starch derivatives.

More preferably, the at least one glidant comprises colloidal silicon dioxide. More preferably, the at least one glidant is colloidal silicon dioxide.

### Lubricant

With regard to the at least one lubricant, no specific restrictions exist provided that the tablet of the invention having the desired dissolution properties are obtained.

Preferably, the at least one lubricant comprises, preferably is, at least one of calcium stearate, glyceryl monostearate, glyceryl palmitostearate, hydrogenated castor oil, hydrogenated vegetable oil, light mineral oil, magnesium stearate, mineral oil, polyethylene glycol, sodium benzoate, sodium lauryl sulfate, sodium stearyl fumarate, stearic acid, talc, zinc stearate, more preferably at least one of calcium stearate, magnesium stearate, polyethylene glycol, sodium stearyl fumarate, stearic acid, talc.

More preferably, the at least one lubricant comprises magnesium stearate. More preferably, the at least one lubricant is magnesium stearate.

### Coating Agent

The tablet may further comprise a coating agent. The coating agent can be formed from an aqueous film coat composition, wherein the aqueous film coat composition may comprise a film-forming polymer, water and/or an alcohol as a vehicle and optionally one or more adjuvants such as are known in the film-coating art. The coating agent may be selected from among hydroxypropylmethylcellulose, hydroxypropylcellulose, methylcellulose, ethylcellulose, hydroxyethylcellulose, cellulose acetate phthalate, sodium ethyl cellulose sulfate, carboxymethyl cellulose, polyvinylpyrrolidone, zein, and an acrylic polymer such as methacrylic acid or methacrylic acid ester copolymers such as methacrylic acid or methylmethacrylate copolymers, and the like, and a polyvinyl alcohol. In particular, the coating agent may comprise a polyvinyl alcohol.

The coating agent may further comprise a taste-masking agent. In this case, the coating agent may be formed from an aqueous film coat composition, wherein the aqueous film coat composition may comprise a film-forming polymer, water and/or an alcohol as a vehicle, and optionally one or more adjuvants such as are known in the film-coating art. The coating agent may be selected from among hydroxypropylmethylcellulose, hydroxypropylcellulose, methylcellulose, ethylcellulose, hydroxyethylcellulose, cellulose acetate phthalate, sodium ethyl cellulose sulfate, carboxymethyl cellulose, polyvinylpyrolidone, zein, and an acrylic polymer such as methacrylic acid or methacrylic acid ester copolymers such as methacrylic acid or methylmethacrylate copolymers, and the like, and a polyvinyl alcohol. In particular, the coating agent may comprise a polyvinyl alcohol.

With respect to the coating agent, film-forming polymers are typically provided in either aqueous or organic solvent-based solutions or aqueous dispersions. However, the polymers may be provided in dry form, alone or in a powdery mixture with other components such as a plasticizer and/or a colorant, which is made into a solution or dispersion by the user by admixing with the aqueous vehicle. It will be appreciated that the aqueous film coat composition may further comprise water as a vehicle for the other components, to facilitate their delivery to the surface of the tablet. The vehicle may optionally further comprise one or more water soluble solvents such as an alcohol and/or a ketone. Examples of an alcohol include but are not limited to methanol, isopropanol, propanol, etc. A non-limiting example for the ketone is acetone. The skilled person can select appropriate vehicle components to provide good interaction between the film-forming polymer and the vehicle to ensure good film properties. In general, polymer-vehicle interaction is designed to yield maximum polymer chain extension to produce films having the greatest cohesive strength and thus mechanical properties. The components are also selected to provide good deposition of the film-forming polymer onto the surface of the tablet, such that a coherent and adherent film is achieved. Suitable aqueous film coating compositions include those commercially available from Colorcon, Inc. of West Point, Pa., under the trade name Opadry® and Opadry® II. Non-limiting examples include Opadry® II Purple and Opadry II® Yellow.

### Composition of the Tablet

Preferably, at least 95 weight-%, preferably at least 97 weight-%, more preferably at least 99 weight-%, more preferably at least 99.5 weight-% of the tablet consist of the polymorphic form of crystalline sofosbuvir according to formula (I) and the at least one pharmaceutically acceptable excipient. More preferably, at least 99.6 weight-%, more preferably at least 99.7 weight-%, more preferably at least 99.8 weight-% of the tablet consist of the polymorphic form of crystalline sofosbuvir according to formula (I) and the at least one pharmaceutically acceptable excipient. More preferably, at least 99.9 weight-% of the tablet consist of the polymorphic form of crystalline sofosbuvir according to formula (I) and the at least one pharmaceutically acceptable excipient.

Preferably, at least 95 weight-%, preferably at least 97 weight-%, more preferably at least 99 weight-%, more preferably at least 99.5 weight-% of the tablet consist of the polymorphic form 6 of crystalline sofosbuvir according to formula (I) and the at least one pharmaceutically acceptable excipient. More preferably, at least 99.6 weight-%, more preferably at least 99.7 weight-%, more preferably at least 99.8 weight-% of the tablet consist of the polymorphic form 6 of crystalline sofosbuvir according to formula (I) and the at least one pharmaceutically acceptable excipient. More preferably, at least 99.9 weight-% of the tablet consist of the polymorphic form 6 of crystalline sofosbuvir according to formula (I) and the at least one pharmaceutically acceptable excipient.

Preferably, at least 95 weight-%, preferably at least 97 weight-%, more preferably at least 99 weight-%, more preferably at least 99.5 weight-% of the tablet consist of the polymorphic form 7 of crystalline sofosbuvir according to formula (I) and the at least one pharmaceutically acceptable excipient. More preferably, at least 99.6 weight-%, more preferably at least 99.7 weight-%, more preferably at least 99.8 weight-% of the tablet consist of the polymorphic form 7 of crystalline sofosbuvir according to formula (I) and the at least one pharmaceutically acceptable excipient. More preferably, at least 99.9 weight-% of the tablet consist of the polymorphic form 7 of crystalline sofosbuvir according to formula (I) and the at least one pharmaceutically acceptable excipient.

Preferably, at least 95 weight-%, preferably at least 97 weight-%, more preferably at least 99 weight-%, more preferably at least 99.5 weight-% of the tablet consist of the polymorphic form of crystalline sofosbuvir according to formula (I) which is a combination of form 6 and form 7 and the at least one pharmaceutically acceptable excipient. More preferably, at least 99.6 weight-%, more preferably at least 99.7 weight-%, more preferably at least 99.8 weight-% of the tablet consist of the polymorphic form of crystalline sofosbuvir according to formula (I) which is a combination of form 6 and form 7 and the at least one pharmaceutically acceptable excipient. More preferably, at least 99.9 weight-% of the tablet consist of the polymorphic form of crystalline sofosbuvir according to formula (I) which is a combination of form 6 and form 7 and the at least one pharmaceutically acceptable excipient.

Preferably, the tablet contains the at least one pharmaceutically acceptable excipient in an amount of at most 75 weight-% or at most 60 weight-%, more preferably in the range of from 55 to 10 weight-%, more preferably from 55 to 15 weight-%, more preferably from 55 to 20 weight-%. More preferably, the tablet contains the at least one pharmaceutically acceptable excipient in an amount in the range of from 50 to 25 weight-%, based on the total weight of the tablet. Preferred ranges include a range of from 50 to 40 weight-% or a range of from 45 to 35 weight-% or a range of from 40 to 30 weight-% or a range of from 35 to 25 weight-% based on the total weight of the tablet. Preferably, the tablet contains the at least one pharmaceutically acceptable excipient in an amount of <25 weight-% based on the total weight of the tablet or in a range of from 25 to 10 weight-% or in a range of from 25 to 15 weight-%, or in a range of from 25 to 20 weight-% or in a range of from 20 to 10 weight-% or in a range of from 15 to 10 weight-% based on the total weight of the tablet.

Preferably, at least 95 weight-%, preferably at least 97 weight-%, more preferably at least 99 weight-%, more preferably at least 99.5 weight-%, more preferably at least 99.6 weight-%, more preferably at least 99.7 weight-%, more preferably at least 99.8 weight-% of the tablet consist of the polymorphic form of crystalline sofosbuvir according to formula (I), mannitol, microcrystalline cellulose, croscarmellose sodium, colloidal silicon dioxide, and magnesium stearate. More preferably, at least 99.9 weight-% of the tablet consist of the polymorphic form of crystalline sofosbuvir according to formula (I), mannitol, microcrystalline cellulose, croscarmellose sodium, colloidal silicon dioxide, and magnesium stearate.

Also preferably, the tablet contains mannitol, microcrystalline cellulose, croscarmellose sodium, colloidal silicon dioxide, and magnesium stearate in an amount of at most 75 weight-% or at most 60 weight-%, more preferably in an amount in in the range of from 55 to 10 weight-%, more preferably from 55 to 15 weight-%, more preferably from 55 to 20 weight-%, more preferably from 50 to 20 weight-%, based on the total weight of the tablet. More preferably, the tablet contains mannitol, microcrystalline cellulose, croscarmellose sodium, colloidal silicon dioxide, and magnesium stearate in an amount in the range of from 50 to 25 weight-% or in an amount of <25 weight-% based on the total weight of the tablet or in a range of from 25 to 10 weight-% or in a range of from 25 to 15 weight-%, or in a range of from 25 to 20 weight-% or in a range of from 20 to 10 weight-% or in a range of from 15 to 10 weight-% based on the total weight of the tablet. More preferably, the tablet contains mannitol, microcrystalline cellulose, croscarmellose sodium, colloidal silicon dioxide, and magnesium stearate in an amount in the range of from 50 to 30 weight-%-% or in an amount of <25 weight-% based on the total weight of the tablet or in a range of from 25 to 10 weight-% or in a range of from 25 to 15 weight-%, or in a range of from 25 to 20 weight-% or in a range of from 20 to 10 weight-% or in a range of from 15 to 10 weight-%, based on the total weight of the tablet. Preferred ranges include a range of from 50 to 40 weight-% or a range of from 45 to 35 weight-% or a range of from 40 to 30 weight-%-% or a range of from 25 to 10 weight-% or a range of from 25 to 15 weight-%, or a range of from 25 to 20 weight-% or a range of from 20 to 10 weight-% or a range of from 15 to 10 weight-% based on the total weight of the tablet.

As mentioned above, it is preferred that the at least one excipient comprises, preferably is, a combination of at least one diluent and at least one disintegrant and at least one glidant and at least one lubricant. In this case, it is preferred that the weight ratio of the at least one disintegrant relative to the at least diluent is in the range of from 0.005:1 to 0.75:1, preferably from 0.01:1 to 0.5:1, more preferably from 0.02:1 to 0.2:1, more preferably from 0.05:1 to 0.1:1. It is further preferred that the weight ratio of the at least one glidant relative to the at least diluent is in the range of from 0.0005:1 to 0.2:1, preferably from 0.001:1 to 0.1:1, more preferably from 0.005:1 to 0.05:1, more preferably from 0.01:1 to 0.02:1. It is further preferred that the weight ratio of the at least one lubricant relative to the at least diluent is in the range of from 0.001:1 to 0.5:1, preferably from 0.005:1 to 0.2:1, more preferably from 0.01:1 to 0.1:1, more preferably from 0.02:1 to 0.05:1.

As mentioned above, it is further preferred that the at least one diluent comprises, preferably is, a combination of mannitol and microcrystalline cellulose, the at least one disintegrant comprises, preferably is, croscarmellose sodium, the at least one glidant comprises, preferably is, colloidal silicon dioxide, and the at least one lubricant comprises, preferably is, magnesium stearate. In this case, it is preferred that the weight ratio of croscarmellose sodium relative to the combination of mannitol and microcrystalline cellulose is in the range of from 0.005:1 to 0.75:1, preferably from 0.01:1 to 0.5:1, more preferably from 0.02:1 to 0.2:1, more preferably from 0.05:1 to 0.1:1. It is further preferred that the weight ratio of colloidal silicon dioxide relative to the combination of mannitol and microcrystalline cellulose is in the range of from 0.0005:1 to 0.2:1, preferably from 0.001:1 to 0.1:1, more preferably from 0.005:1 to 0.05:1, more preferably from 0.01:1 to 0.02:1. It is further preferred that the weight ratio of magnesium stearate relative to the combination of mannitol and microcrystalline cellulose is in the range of from 0.001:1 to 0.5:1, preferably from 0.005:1 to 0.2:1, more preferably from 0.01:1 to 0.1:1, more preferably from 0.02:1 to 0.05:1.

Therefore, the present invention also relates to a tablet comprising a polymorphic form of crystalline sofosbuvir according to formula (I)

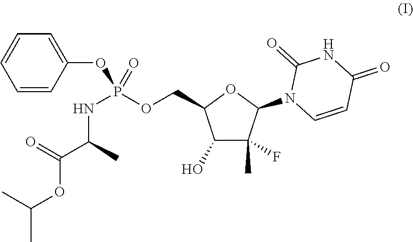 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

in an amount of at least 40 weight-%, preferably ≧75 weight-% based on the total weight of the tablet, wherein the polymorphic form has a moisture stability of at least 95%, wherein the moisture stability of the polymorphic form is defined as the amount of the polymorphic form which is present after an exposure to a relative humidity of (75±1) % at a temperature of (40±1)° C. for an exposure time of (18±1) hours, relative to the amount of the polymorphic form before said exposure, as determined via X-ray powder diffraction pattern analysis at a temperature in the range of from 15 to 25° C. with Cu-Kalpha_{1,2} radiation having a wavelength of 0.15419 nm before the exposure and after the exposure, wherein the polymorphic form of crystalline sofosbuvir according to formula (I) is polymorphic form 6 or polymorphic form 7 or a combination of polymorphic form 6 and polymorphic form 7, wherein the tablet further comprises at most 60 weight-%, preferably <25 weight-% of at least one pharmaceutically acceptable excipient comprising, preferably being, a combination of at least one diluent and at least one disintegrant and at least one glidant and at least one lubricant, more preferably comprising, preferably being, a combination of mannitol, microcrystalline cellulose, croscarmellose sodium, colloidal silicon dioxide, and magnesium stearate

Therefore, the present invention also relates to a tablet comprising a polymorphic form of crystalline sofosbuvir according to formula (I)

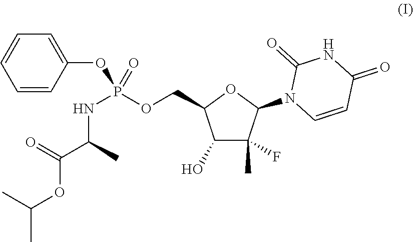 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

in an amount of at least 40 weight-%, preferably ≧75 weight-% based on the total weight of the tablet, wherein the polymorphic form has a moisture stability of at least 95%, wherein the moisture stability of the polymorphic form is defined as the amount of the polymorphic form which is present after an exposure to a relative humidity of (75±1) % at a temperature of (40±1)° C. for an exposure time of (18±1) hours, relative to the amount of the polymorphic form before said exposure, as determined via X-ray powder diffraction pattern analysis at a temperature in the range of from 15 to 25° C. with Cu-Kalpha_{1,2} radiation having a wavelength of 0.15419 nm before the exposure and after the exposure, wherein the polymorphic form of crystalline sofosbuvir according to formula (I) is polymorphic form 6 wherein the tablet further comprises at most 60 weight-%, preferably <25 weight-% of at least one pharmaceutically acceptable excipient comprising, preferably being, a combination of at least one diluent and at least one disintegrant and at least one glidant and at least one lubricant, more preferably comprising, preferably being, a combination of mannitol, microcrystalline cellulose, croscarmellose sodium, colloidal silicon dioxide, and magnesium stearate.

Therefore, the present invention also relates to a tablet comprising a polymorphic form of crystalline sofosbuvir according to formula (I)

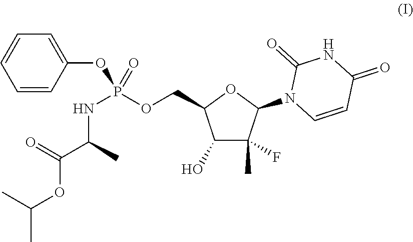 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

in an amount of at least 25 weight-%, preferably at least 40 weight-%, more preferably ≧75 weight-% based on the total weight of the tablet, wherein the polymorphic form has a moisture stability of at least 95%, wherein the moisture stability of the polymorphic form is defined as the amount of the polymorphic form which is present after an exposure to a relative humidity of (75±1) % at a temperature of (40±1)° C. for an exposure time of (18±1) hours, relative to the amount of the polymorphic form before said exposure, as determined via X-ray powder diffraction pattern analysis at a temperature in the range of from 15 to 25° C. with Cu-Kalpha_{1,2} radiation having a wavelength of 0.15419 nm before the exposure and after the exposure, wherein the polymorphic form of crystalline sofosbuvir according to formula (I) is polymorphic form 7 wherein the tablet further comprises at most 75 weight-% or at most 60 weight-%, preferably <25 weight-% of at least one pharmaceutically acceptable excipient comprising, preferably being, a combination of at least one diluent and at least one disintegrant and at least one glidant and at least one lubricant, more preferably comprising, preferably being, a combination of mannitol, microcrystalline cellulose, croscarmellose sodium, colloidal silicon dioxide, and magnesium stearate.

Therefore, the present invention also relates to a tablet comprising a polymorphic form of crystalline sofosbuvir according to formula (I)

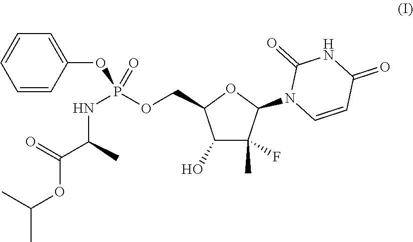 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

in an amount of at least 25 weight-%, preferably at least 40 weight-%, more preferably ≧75 weight-% based on the total weight of the tablet, wherein the polymorphic form has a moisture stability of at least 95%, wherein the moisture stability of the polymorphic form is defined as the amount of the polymorphic form which is present after an exposure to a relative humidity of (75±1) % at a temperature of (40±1)° C. for an exposure time of (18±1) hours, relative to the amount of the polymorphic form before said exposure, as determined via X-ray powder diffraction pattern analysis at a temperature in the range of from 15 to 25° C. with Cu-Kalpha_{1,2} radiation having a wavelength of 0.15419 nm before the exposure and after the exposure, wherein the polymorphic form of crystalline sofosbuvir according to formula (I) is a combination of the polymorphic form 6 and 7 wherein the tablet further comprises at most 75 weight-% or at most 60 weight-%, preferably <25 weight-% of at least one pharmaceutically acceptable excipient comprising, preferably being, a combination of at least one diluent and at least one disintegrant and at least one glidant and at least one lubricant, more preferably comprising, preferably being, a combination of mannitol, microcrystalline cellulose, croscarmellose sodium, colloidal silicon dioxide, and magnesium stearate.

Preferably, the tablet comprises the polymorphic form 6 or the polymorphic form 7 or a combination of polymorphic form 6 and polymorphic form 7 in an amount of ≧75 weight-% or in a range of from 75 to 90 weight-% or in a range of from 75 to 85 weight-%, or in a range of from 75 to 80 weight-% or in a range of from 80 to 90 weight-% or in a range of from 85 to 90 weight-% based on the total weight of the tablet.

Preferably, the tablet comprises the polymorphic form 6 in an amount of ≧75 weight-% or in a range of from 75 to 90 weight-% or in a range of from 75 to 85 weight-%, or in a range of from 75 to 80 weight-% or in a range of from 80 to 90 weight-% or in a range of from 85 to 90 weight-% based on the total weight of the tablet.

The tablet comprises the polymorphic form 7 in an amount of at least 25 weight-%, or at least 30 weight-%, or at least 35 weight % or at least 40 weight % or at least 45 weight-% or in an amount of ≧75 weight-% or in a range of from 75 to 90 weight-% or in a range of from 75 to 85 weight-%, or in a range of from 75 to 80 weight-% or in a range of from 80 to 90 weight-% or in a range of from 85 to 90 weight-% based on the total weight of the tablet.

Preferably, the tablet comprises a combination of polymorphic form 6 and polymorphic form 7 in an amount of ≧75 weight-% or in a range of from 75 to 90 weight-% or in a range of from 75 to 85 weight-%, or in a range of from 75 to 80 weight-% or in a range of from 80 to 90 weight-% or in a range of from 85 to 90 weight-% based on the total weight of the tablet.

Preferably, the tablet comprises the at least one pharmaceutically acceptable excipient in an amount of <25 weight-% or in a range of from 25 to 10 weight-% or in a range of from 25 to 15 weight-%, or in a range of from 25 to 20 weight-% or in a range of from 20 to 10 weight-% or in a range of from 15 to 10 weight-%, based on the total weight of the tablet, wherein the at least one pharmaceutically acceptable excipient comprises preferably is, a combination of at least one diluent and at least one disintegrant and at least one glidant and at least one lubricant, more preferably comprises, preferably is a combination of mannitol, microcrystalline cellulose, croscarmellose sodium, colloidal silicon dioxide, and magnesium stearate.

Preferably, at least 95 weight-%, preferably at least 97 weight-%, more preferably at least 99 weight-%, more preferably at least 99.5 weight-% of the tablet consist of the polymorphic form of crystalline sofosbuvir according to formula (I) and the at least one pharmaceutically acceptable excipient, wherein the polymorphic form is polymorphic form 6 or polymorphic form 7 or a combination of polymorphic form 6 and polymorphic form 7 of crystalline sofosbuvir of formula (I). More preferably, at least 99.6 weight-%, more preferably at least 99.7 weight-%, more preferably at least 99.8 weight-% of the tablet consist of the polymorphic form of crystalline sofosbuvir according to formula (I) and the at least one pharmaceutically acceptable excipient, wherein the polymorphic form is polymorphic form 6 or polymorphic form 7 or a combination of polymorphic form 6 and polymorphic form 7 of crystalline sofosbuvir of formula (I). More preferably, at least 99.9 weight-% of the tablet consist of the polymorphic form of crystalline sofosbuvir according to formula (I) and the at least one pharmaceutically acceptable excipient, wherein the polymorphic form is polymorphic form 6 or polymorphic form 7 or a combination of polymorphic form 6 and polymorphic form 7 of crystalline sofosbuvir of formula (I).

Preferably, at least 95 weight-%, preferably at least 97 weight-%, more preferably at least 99 weight-%, more preferably at least 99.5 weight-% of the tablet consist of the polymorphic form of crystalline sofosbuvir according to formula (I) and the at least one pharmaceutically acceptable excipient, wherein the polymorphic form is polymorphic form 6 of crystalline sofosbuvir of formula (I). More preferably, at least 99.6 weight-%, more preferably at least 99.7 weight-%, more preferably at least 99.8 weight-% of the tablet consist of the polymorphic form of crystalline sofosbuvir according to formula (I) and the at least one pharmaceutically acceptable excipient, wherein the polymorphic form is polymorphic form 6 of crystalline sofosbuvir of formula (I). More preferably, at least 99.9 weight-% of the tablet consist of the polymorphic form of crystalline sofosbuvir according to formula (I) and the at least one pharmaceutically acceptable excipient, wherein the polymorphic form is polymorphic form 6 of crystalline sofosbuvir of formula (I).

Preferably, at least 95 weight-%, preferably at least 97 weight-%, more preferably at least 99 weight-%, more preferably at least 99.5 weight-% of the tablet consist of the polymorphic form of crystalline sofosbuvir according to formula (I) and the at least one pharmaceutically acceptable excipient, wherein the polymorphic form is polymorphic form 7 of crystalline sofosbuvir of formula (I). More preferably, at least 99.6 weight-%, more preferably at least 99.7 weight-%, more preferably at least 99.8 weight-% of the tablet consist of the polymorphic form of crystalline sofosbuvir according to formula (I) and the at least one pharmaceutically acceptable excipient, wherein the polymorphic form is polymorphic form 7 of crystalline sofosbuvir of formula (I). More preferably, at least 99.9 weight-% of the tablet consist of the polymorphic form of crystalline sofosbuvir according to formula (I) and the at least one pharmaceutically acceptable excipient, wherein the polymorphic form is polymorphic form 6 or polymorphic form 7 of crystalline sofosbuvir of formula (I).

Preferably, at least 95 weight-%, preferably at least 97 weight-%, more preferably at least 99 weight-%, more preferably at least 99.5 weight-% of the tablet consist of the polymorphic form of crystalline sofosbuvir according to formula (I) and the at least one pharmaceutically acceptable excipient, wherein the polymorphic form is a combination of polymorphic form 6 and polymorphic form 7 of crystalline sofosbuvir of formula (I). More preferably, at least 99.6 weight-%, more preferably at least 99.7 weight-%, more preferably at least 99.8 weight-% of the tablet consist of the polymorphic form of crystalline sofosbuvir according to formula (I) and the at least one pharmaceutically acceptable excipient, wherein the polymorphic form is a combination of polymorphic form 6 and polymorphic form 7 of crystalline sofosbuvir of formula (I). More preferably, at least 99.9 weight-% of the tablet consist of the polymorphic form of crystalline sofosbuvir according to formula (I) and the at least one pharmaceutically acceptable excipient, wherein the polymorphic form is a combination of polymorphic form 6 and polymorphic form 7 of crystalline sofosbuvir of formula (I).

Thus, the present invention also relates to a tablet comprising a polymorphic form of crystalline sofosbuvir according to formula (I)

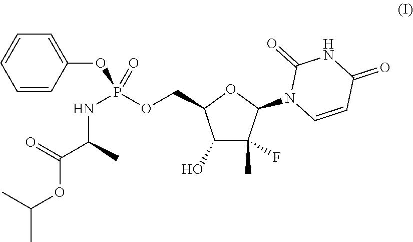 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

in an amount of at least 40 weight-%, preferably ≧75 weight-% based on the total weight of the tablet, wherein the polymorphic form has a moisture stability of at least 95%, wherein the moisture stability of the polymorphic form is defined as the amount of the polymorphic form which is present after an exposure to a relative humidity of (75±1) % at a temperature of (40±1)° C. for an exposure time of (18±1) hours, relative to the amount of the polymorphic form before said exposure, as determined via X-ray powder diffraction pattern analysis at a temperature in the range of from 15 to 25° C. with Cu-Kalpha_{1,2} radiation having a wavelength of 0.15419 nm before the exposure and after the exposure, wherein the polymorphic form of crystalline sofosbuvir according to formula (I) is polymorphic form 6 or polymorphic form 7 or a combination of polymorphic form 6 and polymorphic form 7, wherein the tablet further comprises at most 60 weight-%, preferably <25 weight-% of at least one pharmaceutically acceptable excipient comprising, preferably being, a combination of at least one diluent and at least one disintegrant and at least one glidant and at least one lubricant, more preferably comprising, preferably being, a combination of mannitol, microcrystalline cellulose, croscarmellose sodium, colloidal silicon dioxide, and magnesium stearate, wherein the weight ratio of the at least one disintegrant, preferably croscarmellose sodium, relative to the at least diluent, preferably the combination of mannitol and microcrystalline cellulose, is in the range of from 0.01:1 to 0.5:1, preferably from 0.02:1 to 0.2:1, more preferably from 0.05:1 to 0.1:1, the weight ratio of the at least one glidant, preferably colloidal silica, relative to the at least diluent, preferably the combination of mannitol and microcrystalline cellulose, is in the range of from 0.001:1 to 0.1:1, preferably from 0.005:1 to 0.05:1, more preferably from 0.01:1 to 0.02:1, and the weight ratio of the at least one lubricant, preferably magnesium stearate, relative to the at least diluent, preferably the combination of mannitol and microcrystalline cellulose, is in the range of from 0.005:1 to 0.2:1, preferably from 0.01:1 to 0.1:1, more preferably from 0.02:1 to 0.05:1.

Thus, the present invention also relates to a tablet comprising a polymorphic form of crystalline sofosbuvir according to formula (I)

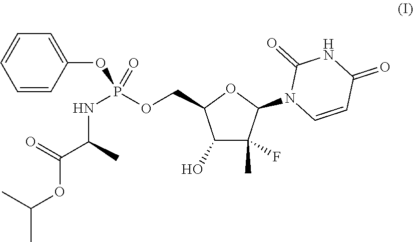 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

in an amount of at least 40 weight-%, preferably ≧75 weight-% based on the total weight of the tablet, wherein the polymorphic form has a moisture stability of at least 95%, wherein the moisture stability of the polymorphic form is defined as the amount of the polymorphic form which is present after an exposure to a relative humidity of (75±1) % at a temperature of (40±1)° C. for an exposure time of (18±1) hours, relative to the amount of the polymorphic form before said exposure, as determined via X-ray powder diffraction pattern analysis at a temperature in the range of from 15 to 25° C. with Cu-Kalpha_{1,2} radiation having a wavelength of 0.15419 nm before the exposure and after the exposure, wherein the polymorphic form of crystalline sofosbuvir according to formula (I) is polymorphic form 6, wherein the tablet further comprises at most 60 weight-%, preferably <25 weight-% of at least one pharmaceutically acceptable excipient comprising, preferably being, a combination of at least one diluent and at least one disintegrant and at least one glidant and at least one lubricant, more preferably comprising, preferably being, a combination of mannitol, microcrystalline cellulose, croscarmellose sodium, colloidal silicon dioxide, and magnesium stearate, wherein the weight ratio of the at least one disintegrant, preferably croscarmellose sodium, relative to the at least diluent, preferably the combination of mannitol and microcrystalline cellulose, is in the range of from 0.01:1 to 0.5:1, preferably from 0.02:1 to 0.2:1, more preferably from 0.05:1 to 0.1:1, the weight ratio of the at least one glidant, preferably colloidal silica, relative to the at least diluent, preferably the combination of mannitol and microcrystalline cellulose, is in the range of from 0.001:1 to 0.1:1, preferably from 0.005:1 to 0.05:1, more preferably from 0.01:1 to 0.02:1, and the weight ratio of the at least one lubricant, preferably magnesium stearate, relative to the at least diluent, preferably the combination of mannitol and microcrystalline cellulose, is in the range of from 0.005:1 to 0.2:1, preferably from 0.01:1 to 0.1:1, more preferably from 0.02:1 to 0.05:1.

Thus, the present invention also relates to a tablet comprising a polymorphic form of crystalline sofosbuvir according to formula (I)

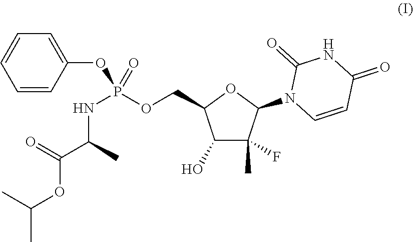 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

in an amount of at least 25 weight-%, of at least 40 weight-%, preferably ≧75 weight-% based on the total weight of the tablet, wherein the polymorphic form has a moisture stability of at least 95%, wherein the moisture stability of the polymorphic form is defined as the amount of the polymorphic form which is present after an exposure to a relative humidity of (75±1) % at a temperature of (40±1)° C. for an exposure time of (18±1) hours, relative to the amount of the polymorphic form before said exposure, as determined via X-ray powder diffraction pattern analysis at a temperature in the range of from 15 to 25° C. with Cu-Kalpha_{1,2} radiation having a wavelength of 0.15419 nm before the exposure and after the exposure, wherein the polymorphic form of crystalline sofosbuvir according to formula (I) is polymorphic form 7, wherein the tablet further comprises at most 60 weight-%, preferably <25 weight-% of at least one pharmaceutically acceptable excipient comprising, preferably being, a combination of at least one diluent and at least one disintegrant and at least one glidant and at least one lubricant, more preferably comprising, preferably being, a combination of mannitol, microcrystalline cellulose, croscarmellose sodium, colloidal silicon dioxide, and magnesium stearate, wherein the weight ratio of the at least one disintegrant, preferably croscarmellose sodium, relative to the at least diluent, preferably the combination of mannitol and microcrystalline cellulose, is in the range of from 0.01:1 to 0.5:1, preferably from 0.02:1 to 0.2:1, more preferably from 0.05:1 to 0.1:1, the weight ratio of the at least one glidant, preferably colloidal silica, relative to the at least diluent, preferably the combination of mannitol and microcrystalline cellulose, is in the range of from 0.001:1 to 0.1:1, preferably from 0.005:1 to 0.05:1, more preferably from 0.01:1 to 0.02:1, and the weight ratio of the at least one lubricant, preferably magnesium stearate, relative to the at least diluent, preferably the combination of mannitol and microcrystalline cellulose, is in the range of from 0.005:1 to 0.2:1, preferably from 0.01:1 to 0.1:1, more preferably from 0.02:1 to 0.05:1.

Thus, the present invention also relates to a tablet comprising a polymorphic form of crystalline sofosbuvir according to formula (I)

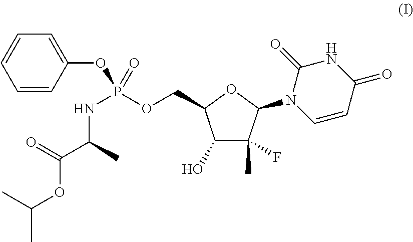 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

in an amount of at least 25 weight-%, of at least 40 weight-%, preferably ≧75 weight-% based on the total weight of the tablet, wherein the polymorphic form has a moisture stability of at least 95%, wherein the moisture stability of the polymorphic form is defined as the amount of the polymorphic form which is present after an exposure to a relative humidity of (75±1) % at a temperature of (40±1)° C. for an exposure time of (18±1) hours, relative to the amount of the polymorphic form before said exposure, as determined via X-ray powder diffraction pattern analysis at a temperature in the range of from 15 to 25° C. with Cu-Kalpha_{1,2} radiation having a wavelength of 0.15419 nm before the exposure and after the exposure, wherein the polymorphic form of crystalline sofosbuvir according to formula (I) is a combination of polymorphic form 6 and polymorphic form 7, wherein the tablet further comprises at most 60 weight-%, preferably <25 weight-% of at least one pharmaceutically acceptable excipient comprising, preferably being, a combination of at least one diluent and at least one disintegrant and at least one glidant and at least one lubricant, more preferably comprising, preferably being, a combination of mannitol, microcrystalline cellulose, croscarmellose sodium, colloidal silicon dioxide, and magnesium stearate, wherein the weight ratio of the at least one disintegrant, preferably croscarmellose sodium, relative to the at least diluent, preferably the combination of mannitol and microcrystalline cellulose, is in the range of from 0.01:1 to 0.5:1, preferably from 0.02:1 to 0.2:1, more preferably from 0.05:1 to 0.1:1, the weight ratio of the at least one glidant, preferably colloidal silica, relative to the at least diluent, preferably the combination of mannitol and microcrystalline cellulose, is in the range of from 0.001:1 to 0.1:1, preferably from 0.005:1 to 0.05:1, more preferably from 0.01:1 to 0.02:1, and the weight ratio of the at least one lubricant, preferably magnesium stearate, relative to the at least diluent, preferably the combination of mannitol and microcrystalline cellulose, is in the range of from 0.005:1 to 0.2:1, preferably from 0.01:1 to 0.1:1, more preferably from 0.02:1 to 0.05:1.

Preferably, the tablet comprises the polymorphic form 6 or the polymorphic form 7 or a combination of polymorphic form 6 and polymorphic form 7 in an amount of ≧75 weight-% based on the total weight of the tablet or in a range of from 75 to 90 weight-% or in a range of from 75 to 85 weight-%, or in a range of from 75 to 80 weight-% or in a range of from 80 to 90 weight-% or in a range of from 85 to 90 weight-% based on the total weight of the tablet.

Preferably, the tablet comprises the polymorphic form 6 in an amount of ≧75 weight-% based on the total weight of the tablet or in a range of from 75 to 90 weight-% or in a range of from 75 to 85 weight-%, or in a range of from 75 to 80 weight-% or in a range of from 80 to 90 weight-% or in a range of from 85 to 90 weight-% based on the total weight of the tablet.

The tablet according to the invention comprises the polymorphic form 7 in an amount of at least 25 weight-%, or at least 30 weight-%, at least 35 weight-%, preferably at least 40% more preferably ≧75 weight-% based on the total weight of the tablet or in a range of from 75 to 90 weight-% or in a range of from 75 to 85 weight-%, or in a range of from 75 to 80 weight-% or in a range of from 80 to 90 weight-% or in a range of from 85 to 90 weight-% based on the total weight of the tablet.

Preferably, the tablet comprises the combination of polymorphic form 6 and polymorphic form 7 in an amount of ≧75 weight-% based on the total weight of the tablet or in a range of from 75 to 90 weight-% or in a range of from 75 to 85 weight-%, or in a range of from 75 to 80 weight-% or in a range of from 80 to 90 weight-% or in a range of from 85 to 90 weight-% based on the total weight of the tablet.

Preferably, the tablet comprises the at least one pharmaceutically acceptable excipient in an amount of <25 weight-% based on the total weight of the tablet or in a range of from 25 to 10 weight-% or in a range of from 25 to 15 weight-%, or in a range of from 25 to 20 weight-% or in a range of from 20 to 10 weight-% or in a range of from 15 to 10 weight-%, based on the total weight of the tablet, wherein the at least one pharmaceutically acceptable excipient comprises, preferably is a combination of at least one diluent and at least one disintegrant and at least one glidant and at least one lubricant, more preferably comprises, preferably is, a combination of mannitol, microcrystalline cellulose, croscarmellose sodium, colloidal silicon dioxide, and magnesium stearate.

Preferably, at least 95 weight-%, preferably at least 97 weight-%, more preferably at least 99 weight-%, more preferably at least 99.5 weight-% of the tablet consist of the polymorphic form of crystalline sofosbuvir according to formula (I) and the at least one pharmaceutically acceptable excipient, wherein the polymorphic form is polymorphic form 6 or polymorphic form 7 or a combination of polymorphic form 6 and polymorphic form 7 of crystalline sofosbuvir of formula (I). More preferably, at least 99.6 weight-%, more preferably at least 99.7 weight-%, more preferably at least 99.8 weight-% of the tablet consist of the polymorphic form of crystalline sofosbuvir according to formula (I) and the at least one pharmaceutically acceptable excipient, wherein the polymorphic form is polymorphic form 6 or polymorphic form 7 or a combination of polymorphic form 6 and polymorphic form 7 of crystalline sofosbuvir of formula (I). More preferably, at least 99.9 weight-% of the tablet consist of the poly-morphic form of crystalline sofosbuvir according to formula (I) and the at least one pharmaceutically acceptable excipient, wherein the polymorphic form is polymorphic form 6 or polymorphic form 7 or a combination of polymorphic form 6 and polymorphic form 7 of crystalline sofosbuvir of formula (I).

Preferably, at least 95 weight-%, preferably at least 97 weight-%, more preferably at least 99 weight-%, more preferably at least 99.5 weight-% of the tablet consist of the polymorphic form 6 of crystalline sofosbuvir according to formula (I) and the at least one pharmaceutically acceptable excipient. More preferably, at least 99.6 weight-%, more preferably at least 99.7 weight-%, more preferably at least 99.8 weight-% of the tablet consist of the polymorphic form 6 of crystalline sofosbuvir according to formula (I) and the at least one pharmaceutically acceptable excipient. More preferably, at least 99.9 weight-% of the tablet consist of the polymorphic form 6 of crystalline sofosbuvir according to formula (I) and the at least one pharmaceutically acceptable excipient.

Preferably, at least 95 weight-%, preferably at least 97 weight-%, more preferably at least 99 weight-%, more preferably at least 99.5 weight-% of the tablet consist of the polymorphic form 7 of crystalline sofosbuvir according to formula (I) and the at least one pharmaceutically acceptable excipient. More preferably, at least 99.6 weight-%, more preferably at least 99.7 weight-%, more preferably at least 99.8 weight-% of the tablet consist of the polymorphic form 7 of crystalline sofosbuvir according to formula (I) and the at least one pharmaceutically acceptable excipient. More preferably, at least 99.9 weight-% of the tablet consist of the polymorphic form 7 of crystalline sofosbuvir according to formula (I) and the at least one pharmaceutically acceptable excipient.

Preferably, at least 95 weight-%, preferably at least 97 weight-%, more preferably at least 99 weight-%, more preferably at least 99.5 weight-% of the tablet consist of the polymorphic form of crystalline sofosbuvir according to formula (I) and the at least one pharmaceutically acceptable excipient, wherein the polymorphic form is a combination of polymorphic form 6 and polymorphic form 7 of crystalline sofosbuvir of formula (I). More preferably, at least 99.6 weight-%, more preferably at least 99.7 weight-%, more preferably at least 99.8 weight-% of the tablet consist of the polymorphic form of crystalline sofosbuvir according to formula (I) and the at least one pharmaceutically acceptable excipient, wherein the polymorphic form is a combination of polymorphic form 6 and polymorphic form 7 of crystalline sofosbuvir of formula (I). More preferably, at least 99.9 weight-% of the tablet consist of the polymorphic form of crystalline sofosbuvir according to formula (I) and the at least one pharmaceutically acceptable excipient, wherein the polymorphic form is a combination of polymorphic form 6 and polymorphic form 7 of crystalline sofosbuvir of formula (I).

Hence, the present invention also relates to a tablet comprising a polymorphic form of crystalline sofosbuvir according to formula (I)

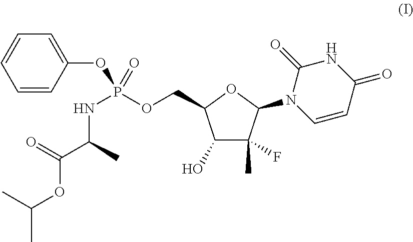 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

in an amount in the range of from 50 to 75 weight-%, preferably in an amount ≧75 weight-% based on the total weight of the tablet, wherein the polymorphic form has a moisture stability of at least 99%, wherein the moisture stability of the polymorphic form is defined as the amount of the polymorphic form which is present after an exposure to a relative humidity of (75±1) % at a temperature of (40±1)° C. for an exposure time of (18±1) hours, relative to the amount of the polymorphic form before said exposure, as determined via X-ray powder diffraction pattern analysis at a temperature in the range of from 15 to 25° C. with Cu-Kalpha_{1,2} radiation having a wavelength of 0.15419 nm before the exposure and after the exposure, wherein the polymorphic form of crystalline sofosbuvir according to formula (I) is polymorphic form 6 or polymorphic form 7 or a combination of polymorphic form 6 and polymorphic form 7, preferably polymorphic form 7, wherein the tablet further comprises at least one pharmaceutically acceptable excipient being a combination of mannitol, microcrystalline cellulose, croscarmellose sodium, colloidal silicon dioxide, and magnesium stearate, wherein the weight ratio of croscarmellose sodium relative to the combination of mannitol and microcrystalline cellulose, is in the range of from 0.05:1 to 0.1:1, the weight ratio of colloidal silica, relative to the combination of mannitol and microcrystalline cellulose is in the range of from 0.01:1 to 0.02:1, and the weight ratio of magnesium stearate relative to the combination of mannitol and microcrystalline cellulose is in the range of from 0.02:1 to 0.05:1.

Hence, the present invention also relates to a tablet comprising a polymorphic form of crystalline sofosbuvir according to formula (I)

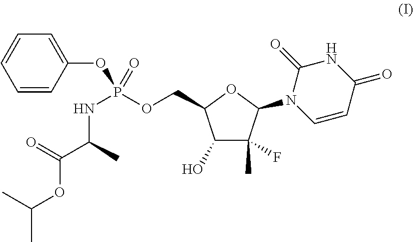 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

in an amount in the range of from 50 to 75 weight-%, preferably in an amount ≧75 weight-% based on the total weight of the tablet, wherein the polymorphic form has a moisture stability of at least 99%, wherein the moisture stability of the polymorphic form is defined as the amount of the polymorphic form which is present after an exposure to a relative humidity of (75±1) % at a temperature of (40±1)° C. for an exposure time of (18±1) hours, relative to the amount of the polymorphic form before said exposure, as determined via X-ray powder diffraction pattern analysis at a temperature in the range of from 15 to 25° C. with Cu-Kalpha_{1,2} radiation having a wavelength of 0.15419 nm before the exposure and after the exposure, wherein the polymorphic form of crystalline sofosbuvir according to formula (I) is polymorphic form 6, wherein the tablet further comprises at least one pharmaceutically acceptable excipient being a combination of mannitol, microcrystalline cellulose, croscarmellose sodium, colloidal silicon dioxide, and magnesium stearate, wherein the weight ratio of croscarmellose sodium relative to the combination of mannitol and microcrystalline cellulose, is in the range of from 0.05:1 to 0.1:1, the weight ratio of colloidal silica, relative to the combination of mannitol and microcrystalline cellulose is in the range of from 0.01:1 to 0.02:1, and the weight ratio of magnesium stearate relative to the combination of mannitol and microcrystalline cellulose is in the range of from 0.02:1 to 0.05:1.

Hence, the present invention also relates to a tablet comprising a polymorphic form of crystalline sofosbuvir according to formula (I)

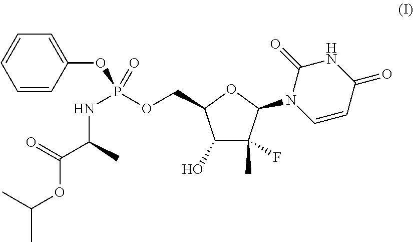 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

in an amount in the range of from 50 to 75 weight-%, preferably in an amount ≧75 weight-% based on the total weight of the tablet, wherein the polymorphic form has a moisture stability of at least 99%, wherein the moisture stability of the polymorphic form is defined as the amount of the polymorphic form which is present after an exposure to a relative humidity of (75±1) % at a temperature of (40±1)° C. for an exposure time of (18±1) hours, relative to the amount of the polymorphic form before said exposure, as determined via X-ray powder diffraction pattern analysis at a temperature in the range of from 15 to 25° C. with Cu-Kalpha_{1,2} radiation having a wavelength of 0.15419 nm before the exposure and after the exposure, wherein the polymorphic form of crystalline sofosbuvir according to formula (I) is polymorphic form 7 wherein the tablet further comprises at least one pharmaceutically acceptable excipient being a combination of mannitol, microcrystalline cellulose, croscarmellose sodium, colloidal silicon dioxide, and magnesium stearate, wherein the weight ratio of croscarmellose sodium relative to the combination of mannitol and microcrystalline cellulose, is in the range of from 0.05:1 to 0.1:1, the weight ratio of colloidal silica, relative to the combination of mannitol and microcrystalline cellulose is in the range of from 0.01:1 to 0.02:1, and the weight ratio of magnesium stearate relative to the combination of mannitol and microcrystalline cellulose is in the range of from 0.02:1 to 0.05:1.

Hence, the present invention also relates to a tablet comprising a polymorphic form of crystalline sofosbuvir according to formula (I)

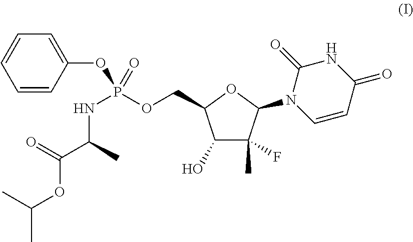 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

in an amount in the range of from 50 to 75 weight-%, preferably in an amount ≧75 weight-% based on the total weight of the tablet, wherein the polymorphic form has a moisture stability of at least 99%, wherein the moisture stability of the polymorphic form is defined as the amount of the polymorphic form which is present after an exposure to a relative humidity of (75±1) % at a temperature of (40±1)° C. for an exposure time of (18±1) hours, relative to the amount of the polymorphic form before said exposure, as determined via X-ray powder diffraction pattern analysis at a temperature in the range of from 15 to 25° C. with Cu-Kalpha_{1,2} radiation having a wavelength of 0.15419 nm before the exposure and after the exposure, wherein the polymorphic form of crystalline sofosbuvir according to formula (I) is a combination of polymorphic form 6 and polymorphic form 7, wherein the tablet further comprises at least one pharmaceutically acceptable excipient being a combination of mannitol, microcrystalline cellulose, croscarmellose sodium, colloidal silicon dioxide, and magnesium stearate, wherein the weight ratio of croscarmellose sodium relative to the combination of mannitol and microcrystalline cellulose, is in the range of from 0.05:1 to 0.1:1, the weight ratio of colloidal silica, relative to the combination of mannitol and microcrystalline cellulose is in the range of from 0.01:1 to 0.02:1, and the weight ratio of magnesium stearate relative to the combination of mannitol and microcrystalline cellulose is in the range of from 0.02:1 to 0.05:1.

Preferably, the tablet comprises the polymorphic form 6 or the polymorphic form 7 or a combination of polymorphic form 6 and polymorphic form 7 in an amount of ≧75 weight-% based on the total weight of the tablet or in a range of from 75 to 90 weight-% or in a range of from 75 to 85 weight-%, or in a range of from 75 to 80 weight-% or in a range of from 80 to 90 weight-% or in a range of from 85 to 90 weight-% based on the total weight of the tablet.

Preferably, the tablet comprises the polymorphic form 6 in an amount of ≧75 weight-% based on the total weight of the tablet or in a range of from 75 to 90 weight-% or in a range of from 75 to 85 weight-%, or in a range of from 75 to 80 weight-% or in a range of from 80 to 90 weight-% or in a range of from 85 to 90 weight-% based on the total weight of the tablet.

Preferably, the tablet comprises the polymorphic form 7 in an amount of ≧75 weight-% based on the total weight of the tablet or in a range of from 75 to 90 weight-% or in a range of from 75 to 85 weight-%, or in a range of from 75 to 80 weight-% or in a range of from 80 to 90 weight-% or in a range of from 85 to 90 weight-% based on the total weight of the tablet.

Preferably, the tablet comprises a combination of polymorphic form 6 and polymorphic form 7 in an amount of ≧75 weight-% based on the total weight of the tablet or in a range of from 75 to 90 weight-% or in a range of from 75 to 85 weight-%, or in a range of from 75 to 80 weight-% or in a range of from 80 to 90 weight-% or in a range of from 85 to 90 weight-% based on the total weight of the tablet.

Preferably, the tablet comprises the at least one pharmaceutically acceptable excipient in an amount of <25 weight-% based on the total weight of the tablet or in a range of from 25 to 10 weight-% or in a range of from 25 to 15 weight-%, or in a range of from 25 to 20 weight-% or in a range of from 20 to 10 weight-% or in a range of from 15 to 10 weight-%, based on the total weight of the tablet, wherein the at least one pharmaceutically acceptable excipient comprises, preferably is a combination of at least one diluent and at least one disintegrant and at least one glidant and at least one lubricant, more preferably comprising, preferably being, a combination of mannitol, microcrystalline cellulose, croscarmellose sodium, colloidal silicon dioxide, and magnesium stearate.

Preferably, at least 95 weight-%, preferably at least 97 weight-%, more preferably at least 99 weight-%, more preferably at least 99.5 weight-% of the tablet consist of the polymorphic form of crystalline sofosbuvir according to formula (I) and the at least one pharmaceutically acceptable excipient, wherein the polymorphic form is polymorphic form 6 or polymorphic form 7 or a combination of polymorphic form 6 and polymorphic form 7 of crystalline sofosbuvir of formula (I). More preferably, at least 99.6 weight-%, more preferably at least 99.7 weight-%, more preferably at least 99.8 weight-% of the tablet consist of the polymorphic form of crystalline sofosbuvir according to formula (I) and the at least one pharmaceutically acceptable excipient, wherein the polymorphic form is polymorphic form 6 or polymorphic form 7 or a combination of polymorphic form 6 and polymorphic form 7 of crystalline sofosbuvir of formula (I). More preferably, at least 99.9 weight-% of the tablet consist of the polymorphic form of crystalline sofosbuvir according to formula (I) and the at least one pharmaceutically acceptable excipient, wherein the polymorphic form is polymorphic form 6 or polymorphic form 7 or a combination of polymorphic form 6 and polymorphic form 7 of crystalline sofosbuvir of formula (I).

Preferably, at least 95 weight-%, preferably at least 97 weight-%, more preferably at least 99 weight-%, more preferably at least 99.5 weight-% of the tablet consist of the polymorphic form 6 of crystalline sofosbuvir according to formula (I) and the at least one pharmaceutically acceptable excipient. More preferably, at least 99.6 weight-%, more preferably at least 99.7 weight-%, more preferably at least 99.8 weight-% of the tablet consist of the polymorphic form 6 of crystalline sofosbuvir according to formula (I) and the at least one pharmaceutically acceptable excipient. More preferably, at least 99.9 weight-% of the tablet consist of the polymorphic form 6 of crystalline sofosbuvir according to formula (I) and the at least one pharmaceutically acceptable excipient.

Preferably, at least 95 weight-%, preferably at least 97 weight-%, more preferably at least 99 weight-%, more preferably at least 99.5 weight-% of the tablet consist of the polymorphic form 7 of crystalline sofosbuvir according to formula (I) and the at least one pharmaceutically acceptable excipient. More preferably, at least 99.6 weight-%, more preferably at least 99.7 weight-%, more preferably at least 99.8 weight-% of the tablet consist of the polymorphic form 7 of crystalline sofosbuvir according to formula (I) and the at least one pharmaceutically acceptable excipient. More preferably, at least 99.9 weight-% of the tablet consist of the polymorphic form 7 of crystalline sofosbuvir according to formula (I) and the at least one pharmaceutically acceptable excipient.

Preferably, at least 95 weight-%, preferably at least 97 weight-%, more preferably at least 99 weight-%, more preferably at least 99.5 weight-% of the tablet consist of the polymorphic form of crystalline sofosbuvir according to formula (I) and the at least one pharmaceutically acceptable excipient, wherein the polymorphic form is a combination of polymorphic form 6 and polymorphic form 7 of crystalline sofosbuvir of formula (I). More preferably, at least 99.6 weight-%, more preferably at least 99.7 weight-%, more preferably at least 99.8 weight-% of the tablet consist of the polymorphic form of crystalline sofosbuvir according to formula (I) and the at least one pharmaceutically acceptable excipient, wherein the polymorphic form is a combination of polymorphic form 6 and polymorphic form 7 of crystalline sofosbuvir of formula (I). More preferably, at least 99.9 weight-% of the tablet consist of the polymorphic form of crystalline sofosbuvir according to formula (I) and the at least one pharmaceutically acceptable excipient, wherein the polymorphic form is a combination of polymorphic form 6 and polymorphic form 7 of crystalline sofosbuvir of formula (I).

Hence, preferably the tablet of the invention is a tablet comprising a polymorphic form of crystalline sofosbuvir according to formula (I)

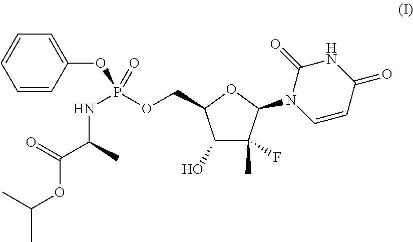 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

in an amount ≧75 weight-% based on the total weight of the tablet, wherein the polymorphic form has a moisture stability of at least 99%, wherein the moisture stability of the polymorphic form is defined as the amount of the polymorphic form which is present after an exposure to a relative humidity of (75±1) % at a temperature of (40±1)° C. for an exposure time of (18±1) hours, relative to the amount of the polymorphic form before said exposure, as determined via X-ray powder diffraction pattern analysis at a temperature in the range of from 15 to 25° C. with Cu-Kalpha_{1,2} radiation having a wavelength of 0.15419 nm before the exposure and after the exposure, wherein the polymorphic form of crystalline sofosbuvir according to formula (I) is polymorphic form 6 or polymorphic form 7 or a combination of polymorphic form 6 and polymorphic form 7, preferably polymorphic form 7, wherein the tablet further comprises at least one pharmaceutically acceptable excipient and wherein at least 95 weight-%, preferably at least 97 weight-%, more preferably at least 99 weight-%, more preferably at least 99.5 weight-% of the tablet consist of the polymorphic form of crystalline sofosbuvir, and the at least one pharmaceutically acceptable excipient.

Preferably, the tablet comprises the at least one pharmaceutically acceptable excipient in an amount of <25 weight-% based on the total weight of the tablet or in a range of from 25 to 10 weight-% or in a range of from 25 to 15 weight-%, or in a range of from 25 to 20 weight-% or in a range of from 20 to 10 weight-% or in a range of from 15 to 10 weight-%, based on the total weight of the tablet, wherein the at least one pharmaceutically acceptable excipient comprises, preferably is a combination of at least one diluent and at least one disintegrant and at least one glidant and at least one lubricant, more preferably comprises, preferably is, a combination of mannitol, microcrystalline cellulose, croscarmellose sodium, colloidal silicon dioxide, and magnesium stearate.

Hence, preferably the tablet of the invention is a tablet comprising a polymorphic form of crystalline sofosbuvir according to formula (I)

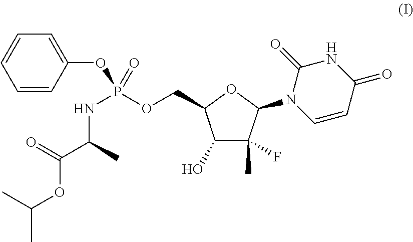 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

in an amount ≧75 weight-% based on the total weight of the tablet, wherein the polymorphic form has a moisture stability of at least 99%, wherein the moisture stability of the polymorphic form is defined as the amount of the polymorphic form which is present after an exposure to a relative humidity of (75±1) % at a temperature of (40±1)° C. for an exposure time of (18±1) hours, relative to the amount of the polymorphic form before said exposure, as determined via X-ray powder diffraction pattern analysis at a temperature in the range of from 15 to 25° C. with Cu-Kalpha_{1,2} radiation having a wavelength of 0.15419 nm before the exposure and after the exposure, wherein the polymorphic form of crystalline sofosbuvir according to formula (I) is polymorphic form 6, wherein the tablet further comprises at least one pharmaceutically acceptable excipient and wherein at least 95 weight-%, preferably at least 97 weight-%, more preferably at least 99 weight-%, more preferably at least 99.5 weight-% of the tablet consist of the polymorphic form 6 of crystalline sofosbuvir, and the at least one pharmaceutically acceptable excipient.

Hence, preferably the tablet of the invention is a tablet comprising a polymorphic form of crystalline sofosbuvir according to formula (I)

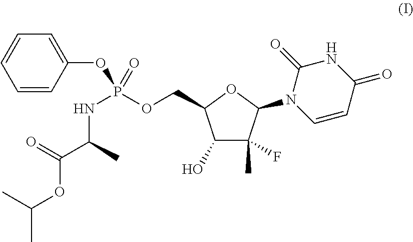 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

in an amount ≧75 weight-% based on the total weight of the tablet, wherein the polymorphic form has a moisture stability of at least 99%, wherein the moisture stability of the polymorphic form is defined as the amount of the polymorphic form which is present after an exposure to a relative humidity of (75±1) % at a temperature of (40±1)° C. for an exposure time of (18±1) hours, relative to the amount of the polymorphic form before said exposure, as determined via X-ray powder diffraction pattern analysis at a temperature in the range of from 15 to 25° C. with Cu-Kalpha_{1,2} radiation having a wavelength of 0.15419 nm before the exposure and after the exposure, wherein the polymorphic form of crystalline sofosbuvir according to formula (I) is polymorphic form 7 wherein the tablet further comprises at least one pharmaceutically acceptable excipient and wherein at least 95 weight-%, preferably at least 97 weight-%, more preferably at least 99 weight-%, more preferably at least 99.5 weight-% of the tablet consist of the polymorphic form 7 of crystalline sofosbuvir, and the at least one pharmaceutically acceptable excipient.

Hence, preferably the tablet of the invention is a tablet comprising a polymorphic form of crystalline sofosbuvir according to formula (I)

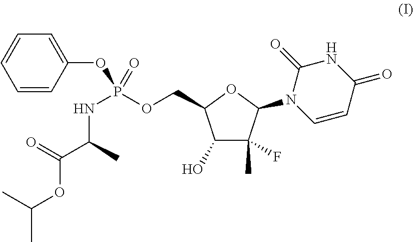 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

in an amount ≧75 weight-% based on the total weight of the tablet, wherein the polymorphic form has a moisture stability of at least 99%, wherein the moisture stability of the polymorphic form is defined as the amount of the polymorphic form which is present after an exposure to a relative humidity of (75±1) % at a temperature of (40±1)° C. for an exposure time of (18±1) hours, relative to the amount of the polymorphic form before said exposure, as determined via X-ray powder diffraction pattern analysis at a temperature in the range of from 15 to 25° C. with Cu-Kalpha_{1,2} radiation having a wavelength of 0.15419 nm before the exposure and after the exposure, wherein the polymorphic form of crystalline sofosbuvir according to formula (I) is a combination of polymorphic form 6 and polymorphic form 7, preferably polymorphic form 7, wherein the tablet further comprises at least one pharmaceutically acceptable excipient and wherein at least 95 weight-%, preferably at least 97 weight-%, more preferably at least 99 weight-%, more preferably at least 99.5 weight-% of the tablet consist of the polymorphic form of crystalline sofosbuvir, and the at least one pharmaceutically acceptable excipient.

Preferably, at least 95 weight-%, preferably at least 97 weight-%, more preferably at least 99 weight-%, more preferably at least 99.5 weight-% of the tablet consist of the polymorphic form of crystalline sofosbuvir according to formula (I) and the at least one pharmaceutically acceptable excipient, wherein the polymorphic form is polymorphic form 6 or polymorphic form 7 or a combination of polymorphic form 6 and polymorphic form 7 of crystalline sofosbuvir of formula (I). More preferably, at least 99.6 weight-%, more preferably at least 99.7 weight-%, more preferably at least 99.8 weight-% of the tablet consist of the polymorphic form of crystalline sofosbuvir according to formula (I) and the at least one pharmaceutically acceptable excipient, wherein the polymorphic form is polymorphic form 6 or polymorphic form 7 or a combination of polymorphic form 6 and polymorphic form 7 of crystalline sofosbuvir of formula (I). More preferably, at least 99.9 weight-% of the tablet consist of the polymorphic form of crystalline sofosbuvir according to formula (I) and the at least one pharmaceutically acceptable excipient, wherein the polymorphic form is polymorphic form 6 or polymorphic form 7 or a combination of polymorphic form 6 and polymorphic form 7 of crystalline sofosbuvir of formula (I).

Preferably, at least 95 weight-%, preferably at least 97 weight-%, more preferably at least 99 weight-%, more preferably at least 99.5 weight-% of the tablet consist of the polymorphic form 6 of crystalline sofosbuvir according to formula (I) and the at least one pharmaceutically acceptable excipient. More preferably, at least 99.6 weight-%, more preferably at least 99.7 weight-%, more preferably at least 99.8 weight-% of the tablet consist of the polymorphic form 6 of crystalline sofosbuvir according to formula (I) and the at least one pharmaceutically acceptable excipient. More preferably, at least 99.9 weight-% of the tablet consist of the polymorphic form 6 of crystalline sofosbuvir according to formula (I) and the at least one pharmaceutically acceptable excipient.

Preferably, at least 95 weight-%, preferably at least 97 weight-%, more preferably at least 99 weight-%, more preferably at least 99.5 weight-% of the tablet consist of the polymorphic form 7 of crystalline sofosbuvir according to formula (I) and the at least one pharmaceutically acceptable excipient. More preferably, at least 99.6 weight-%, more preferably at least 99.7 weight-%, more preferably at least 99.8 weight-% of the tablet consist of the polymorphic form 7 of crystalline sofosbuvir according to formula (I) and the at least one pharmaceutically acceptable excipient. More preferably, at least 99.9 weight-% of the tablet consist of the polymorphic form 7 of crystalline sofosbuvir according to formula (I) and the at least one pharmaceutically acceptable excipient.

Preferably, at least 95 weight-%, preferably at least 97 weight-%, more preferably at least 99 weight-%, more preferably at least 99.5 weight-% of the tablet consist of the polymorphic form of crystalline sofosbuvir according to formula (I) and the at least one pharmaceutically acceptable excipient, wherein the polymorphic form is a combination of polymorphic form 6 and polymorphic form 7 of crystalline sofosbuvir of formula (I). More preferably, at least 99.6 weight-%, more preferably at least 99.7 weight-%, more preferably at least 99.8 weight-% of the tablet consist of the polymorphic form of crystalline sofosbuvir according to formula (I) and the at least one pharmaceutically acceptable excipient, wherein the polymorphic form is a combination of polymorphic form 6 and polymorphic form 7 of crystalline sofosbuvir of formula (I). More preferably, at least 99.9 weight-% of the tablet consist of the polymorphic form of crystalline sofosbuvir according to formula (I) and the at least one pharmaceutically acceptable excipient, wherein the polymorphic form is a combination of polymorphic form 6 and polymorphic form 7 of crystalline sofosbuvir of formula (I).

The tablet of the present invention may have a mass in the range of from 0.3 to 2.0 g, preferably from 0.4 to 1.5 g. More preferably, the tablet has a mass in the range of from 0.45 to 1.00 g, more preferably from 0.455 to 0.70 g, more preferably from 0.50 to 0.80 g.

Further, the tablet of the invention is preferably a round or an oval tablet, having a longest dimension of at most 20 mm, preferably at most 19, more preferably at most 18 mm. Longest dimensions of at most 17 or of at most 16 mm or of at most 15 mm are also conceivable.

### Dissolution Profile

The tablet of the present invention has advantageous dissolution characteristics.

preferably, the tablet exhibits a dissolution of at least 40%, preferably at least 45%, more preferably at least 50%, more preferably at least 55%, more preferably at least 60%, after 5 minutes in the mean dissolution profile as determined with a USP dissolution paddle apparatus 2 at 37° C., 75 r.p.m., using a phosphate buffer having a pH of 6.8 and a vessel having a volume of 900 ml, described in Reference Example 8 hereinbelow.

Preferably, the tablet exhibits a dissolution of at least 60%, preferably at least 65%, more preferably at least 70%, more preferably at least 75%, more preferably at least 80%, after 10 minutes in the mean dissolution profile as determined with a USP dissolution paddle apparatus 2 at 37° C., 75 r.p.m., using a phosphate buffer having a pH of 6.8 and a vessel having a volume of 900 ml, described in Reference Example 8 hereinbelow.

Preferably, the tablet exhibits a dissolution of at least 80%, preferably at least 85%, more preferably at least 90%, more preferably at least 95%, after 15 minutes in the mean dissolution profile as determined with a USP dissolution paddle apparatus 2 at 37° C., 75 r.p.m., using a phosphate buffer having a pH of 6.8 and a vessel having a volume of 900 ml, described in Reference Example 8 hereinbelow.

More preferably, the tablet exhibits a dissolution of at least 60% after 5 minute, of at least 80% after 10 minutes, and of at least 90% after 15 minutes in the mean dissolution profile as determined with a USP dissolution paddle apparatus 2 at 37° C., 75 r.p.m., using a phosphate buffer having a pH of 6.8 and a vessel having a volume of 900 ml, described in Reference Example 8 hereinbelow.

More preferably, the tablet of at least 60% after 5 minute, of at least 80% after 10 minutes, and of at least 95% after 15 minutes in the mean dissolution profile as determined with a USP dissolution paddle apparatus 2 at 37° C., 75 r.p.m., using a phosphate buffer having a pH of 6.8 and a vessel having a volume of 900 ml, described in Reference Example 8 hereinbelow.

Therefore, the present invention also relates to a tablet comprising a polymorphic form of crystalline sofosbuvir according to formula (I)

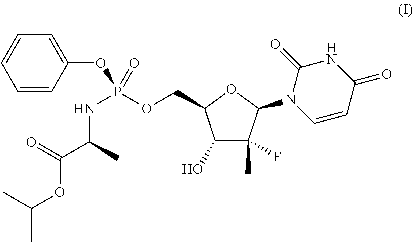 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

in an amount in the range of from 50 to 75 weight-%, preferably in an amount ≧75 weight-% based on the total weight of the tablet, wherein the polymorphic form has a moisture stability of at least 99%, wherein the moisture stability of the polymorphic form is defined as the amount of the polymorphic form which is present after an exposure to a relative humidity of (75±1) % at a temperature of (40±1)° C. for an exposure time of (18±1) hours, relative to the amount of the polymorphic form before said exposure, as determined via X-ray powder diffraction pattern analysis at a temperature in the range of from 15 to 25° C. with Cu-Kalpha_{1,2} radiation having a wavelength of 0.15419 nm before the exposure and after the exposure, wherein the polymorphic form of crystalline sofosbuvir according to formula (I) is polymorphic form 6 or polymorphic form 7 or a combination of polymorphic form 6 and polymorphic form 7, preferably polymorphic form 7, wherein the tablet further comprises at least one pharmaceutically acceptable excipient being a combination of mannitol, microcrystalline cellulose, croscarmellose sodium, colloidal silicon dioxide, and magnesium stearate, wherein the weight ratio of croscarmellose sodium relative to the combination of mannitol and microcrystalline cellulose, is in the range of from 0.05:1 to 0.1:1, the weight ratio of colloidal silica, relative to the combination of mannitol and microcrystalline cellulose is in the range of from 0.01:1 to 0.02:1, and the weight ratio of magnesium stearate relative to the combination of mannitol and microcrystalline cellulose is in the range of from 0.02:1 to 0.05:1, wherein the tablet exhibits a dissolution of at least 60% after 5 minute, of at least 80% after 10 minutes, and of at least 90% after 15 minutes in the mean dissolution profile as determined with a USP dissolution paddle apparatus 2 at 37° C., 75 r.p.m., using a phosphate buffer having a pH of 6.8 and a vessel having a volume of 900 ml, described in Reference Example 8 hereinbelow, and wherein the tablet has a mass in the range of from 0.50 to 0.80 g.

### Process for the Preparation of the Tablet

Generally, the tablet of the present invention can be prepared by all conceivable processes. Preferably, as mentioned above, the tablet of the present invention comprises at least one pharmaceutically acceptable excipient. The choice of particular types and amounts of excipients, and tableting technique employed generally depends on the properties of the polymorphic form of sofosbuvir and the excipients such as compressibility, flowability, particle size, compatibility, and density. In this regard, reference is made Remington: The Science and Practice of Pharmacy 2006, 21^{st} edition, Lippincott Williams & Wilkins. Reference is also made to the Handbook of Pharmaceutical Excipients 1994, edited by A. Wade and P. J. Weller, The Pharmaceutical Press, 2^{nd} Edition, London. The tablets may be prepared according to methods known in the art, including dry granulation, such as via roller compaction, wet granulation, such as via fluid bed granulation and high shear granulation, or direct compression, and the type of excipients used will vary accordingly. Therefore, the present invention relates to a process for preparing the tablet of the invention, comprising
(a) providing the polymorphic form of crystalline sofosbuvir according to formula (I),

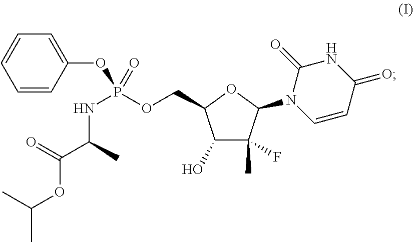 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

(b) blending the polymorphic form provided in (a) with at least one pharmaceutically acceptable excipient;
(c) preparing the tablet based on the blend obtained in (b).

Dry granulation is preferred for preparing the tablets of the invention. A preferred dry granu-lated tablet comprises granules comprising the polymorphic form of sofosbuvir and at least one diluent and/or at least one disintegrant and/or at least one glidant and/or at least one lubricant, preferably a combination of at least one diluent and at least one disintegrant and at least one glidant and at least one lubricant, more preferably a combination of mannitol, microcrystalline cellulose, croscarmellose sodium, colloidal silicon dioxide, and magnesium stearate, wherein the granules are mixed with at least one diluent and/or at least one disintegrant and/or at least one glidant and/or at least one lubricant, preferably with a combination of at least one diluent and at least one disintegrant and at least one glidant and at least one lubricant, more preferably with a combination of microcrystalline cellulose, croscarmellose sodium, colloidal silicon dioxide, and magnesium stearate.

Therefore, the present invention also relates to the process as described above, wherein (b) comprises
(b.1) blending the polymorphic form provided in (a) with a first portion of the at least one pharmaceutically acceptable excipient;
(b.2) compressing the blend obtained in (b.1), obtaining a molding, preferably a tablet;
(b.3) crushing the molding, preferably the tablet, obtained in (b.2), preferably over a sieve;
(b.4) blending the particles obtained in (b.3) with a second portion of the at least one pharmaceutically acceptable excipient;
   and wherein (c) comprises
(c) compressing the blend obtained in (b.4), obtaining the tablet.

Preferably, the total weight contained in the first portion in (b.1) relative to the total weight of the at least one pharmaceutically acceptable excipient contained in the first portion in (b.1) and the second portion in (b.4) is in the range of from 30 to 99%, more preferably from 35 to 98%, more preferably from 40 to 97%, more preferably from 45 to 96%. More preferably, the total weight of the at least one pharmaceutically acceptable excipient contained in the first portion in (b.1) relative to the total weight of the at least one pharmaceutically acceptable excipient contained in the first portion in (b.1) and the second portion in (b.4) is in the range of from 50 to 95%, more preferably from 55 to 94%, more preferably from 60 to 93%, more preferably from 65 to 92%, more preferably from 70 to 90%. Preferred ranges include a range of from 70 to 80% or from 75 to 85% or from 80 to 90%.

Preferably, the first portion in (b.1) comprises, preferably is, a combination of mannitol, microcrystalline cellulose, croscarmellose sodium, colloidal silicon dioxide, and magnesium stearate, and the second portion in (b.2) comprises, preferably is, a combination of microcrystalline cellulose, croscarmellose sodium, colloidal silicon dioxide, and magnesium stearate.

Preferably, the compressing in (b.2) is carried out under a pressure in the range of from 50 to 250 bar, more preferably from 75 to 200 bar, more preferably from 100 to 150 bar. Preferred ranges include a range of from 100 to 120 bar or a range of from 110 to 130 bar or a range of from 120 to 140 bar or a range of from 130 to 150 bar.

Preferably, in (b.3), the sieve has mesh size in the range of from 0.5 to 1.5 mm, more preferably from 0.7 to 1.2 mm, more preferably from 0.75 to 1.25 mm. Preferred ranges include a range of from 0.75 to 0.95 mm or a range of from 0.85 to 1.05 mm or a range of from 0.95 to 1.15 mm or a range of from 1.05 to 1.25 mm.

Preferably, the compressing in (c) is carried out under a pressure in the range of from 50 to 300 bar, more preferably from 75 to 275 bar, preferably from 100 to 250 bar. Preferred ranges include a range of from 100 to 150 bar or a range of from 125 to 175 bar or a range of from 150 to 200 bar or a range of from 175 to 225 bar or a range of from 200 to 250 bar.

Preferably, the tablet of (c) comprises the polymorphic form in an amount of ≧75 weight-% based on the total weight of the tablet or in a range of from 75 to 90 weight-% or in a range of from 75 to 85 weight-%, or in a range of from 75 to 80 weight-% or in a range of from 80 to 90 weight-% or in a range of from 85 to 90 weight-% based on the total weight of the tablet.

Preferably, the tablet of (c) comprises the polymorphic form in an amount of ≧75 weight-% based on the total weight of the tablet or in a range of from 75 to 90 weight-% or in a range of from 75 to 85 weight-%, or in a range of from 75 to 80 weight-% or in a range of from 80 to 90 weight-% or in a range of from 85 to 90 weight-% based on the total weight of the tablet wherein the polymorphic form is polymorphic form 6 or polymorphic form 7 or a combination of polymorphic form 6 and polymorphic form 7 of crystalline sofosbuvir of formula (I). Preferably, the tablet of (c) comprises the polymorphic form 6 in an amount of ≧75 weight-% based on the total weight of the tablet or in a range of from 75 to 90 weight-% or in a range of from 75 to 85 weight-%, or in a range of from 75 to 80 weight-% or in a range of from 80 to 90 weight-% or in a range of from 85 to 90 weight-% based on the total weight of the tablet.

Preferably, the tablet of (c) comprises the polymorphic form 7 in an amount of ≧75 weight-% based on the total weight of the tablet or in a range of from 75 to 90 weight-% or in a range of from 75 to 85 weight-%, or in a range of from 75 to 80 weight-% or in a range of from 80 to 90 weight-% or in a range of from 85 to 90 weight-% based on the total weight of the tablet.

It is conceivable, that the tablet of (c) comprises the polymorphic form 7 in an amount of weight-at least 25 weight-%, of at least 30 weight-% of at least 35-weight-% of at least 40 weight-% based on the total weight of the tablet.

Preferably, the tablet of (c) comprises the polymorphic form in an amount of ≧75 weight-% based on the total weight of the tablet or in a range of from 75 to 90 weight-% or in a range of from 75 to 85 weight-%, or in a range of from 75 to 80 weight-% or in a range of from 80 to 90 weight-% or in a range of from 85 to 90 weight-% based on the total weight of the tablet wherein the polymorphic form is a combination of polymorphic form 6 and polymorphic form 7 of crystalline sofosbuvir of formula (I).

Preferably, at least 95 weight-%, preferably at least 97 weight-%, more preferably at least 99 weight-%, more preferably at least 99.5 weight-% of the tablet of (c) consist of the polymorphic form of crystalline sofosbuvir according to formula (I) and the at least one pharmaceutically acceptable excipient, wherein the polymorphic form is polymorphic form 6 or polymorphic form 7 or a combination of polymorphic form 6 and polymorphic form 7 of crystalline sofosbuvir of formula (I). More preferably, at least 99.6 weight-%, more preferably at least 99.7 weight-%, more preferably at least 99.8 weight-% of the tablet consist of the polymorphic form of crystalline sofosbuvir according to formula (I) and the at least one pharmaceutically acceptable excipient, wherein the polymorphic form is polymorphic form 6 or polymorphic form 7 or a combination of polymorphic form 6 and polymorphic form 7 of crystalline sofosbuvir of formula (I). More preferably, at least 99.9 weight-% of the tablet consist of the polymorphic form of crystalline sofosbuvir according to formula (I) and the at least one pharmaceutically acceptable excipient, wherein the polymorphic form is polymorphic form 6 or polymorphic form 7 or a combination of polymorphic form 6 and polymorphic form 7 of crystalline sofosbuvir of formula (I).

Preferably, at least 95 weight-%, preferably at least 97 weight-%, more preferably at least 99 weight-%, more preferably at least 99.5 weight-% of the tablet of (c) consist of the polymorphic form 6 of crystalline sofosbuvir according to formula (I) and the at least one pharmaceutically acceptable excipient. More preferably, at least 99.6 weight-%, more preferably at least 99.7 weight-%, more preferably at least 99.8 weight-% of the tablet consist of the polymorphic form 6 of crystalline sofosbuvir according to formula (I) and the at least one pharmaceutically acceptable excipient. More preferably, at least 99.9 weight-% of the tablet consist of the polymorphic form 6 of crystalline sofosbuvir according to formula (I) and the at least one pharmaceutically acceptable excipient.

Preferably, at least 95 weight-%, preferably at least 97 weight-%, more preferably at least 99 weight-%, more preferably at least 99.5 weight-% of the tablet of (c) consist of the polymorphic form 7 of crystalline sofosbuvir according to formula (I) and the at least one pharmaceutically acceptable excipient. More preferably, at least 99.6 weight-%, more preferably at least 99.7 weight-%, more preferably at least 99.8 weight-% of the tablet consist of the polymorphic form 7 of crystalline sofosbuvir according to formula (I) and the at least one pharmaceutically acceptable excipient. More preferably, at least 99.9 weight-% of the tablet consist of the polymorphic form 7 of crystalline sofosbuvir according to formula (I) and the at least one pharmaceutically acceptable excipient.

Preferably, at least 95 weight-%, preferably at least 97 weight-%, more preferably at least 99 weight-%, more preferably at least 99.5 weight-% of the tablet of (c) consist of the polymorphic form of crystalline sofosbuvir according to formula (I) and the at least one pharmaceutically acceptable excipient, wherein the polymorphic form is a combination of polymorphic form 6 and polymorphic form 7 of crystalline sofosbuvir of formula (I). More preferably, at least 99.6 weight-%, more preferably at least 99.7 weight-%, more preferably at least 99.8 weight-% of the tablet consist of the polymorphic form of crystalline sofosbuvir according to formula (I) and the at least one pharmaceutically acceptable excipient, wherein the polymorphic form is a combination of polymorphic form 6 and polymorphic form 7 of crystalline sofosbuvir of formula (I). More preferably, at least 99.9 weight-% of the tablet consist of the polymorphic form of crystalline sofosbuvir according to formula (I) and the at least one pharmaceutically acceptable excipient, wherein the polymorphic form is a combination of polymorphic form 6 and polymorphic form 7 of crystalline sofosbuvir of formula (I). As mentioned above, generally no specific restrictions exists how a polymorphic form exhibiting the moisture stability as defined above is provided or prepared. Preferably, the polymorphic form is obtainable or obtained by a process comprising crystallization of the polymorphic form in the presence of at least one organic solvent and preferably at least one organic anti-solvent.

Therefore, the present invention relates to the process as defined above, wherein providing the polymorphic form of crystalline sofosbuvir according to formula (I) in (a) comprises crystallizing the polymorphic form in the presence of at least one organic solvent and preferably at least one organic anti-solvent.

Further, the present invention relates to the process as defined above, wherein the polymorphic form of crystalline sofosbuvir according to formula (I) provided in (a) is obtainable or obtained by a process comprising crystallizing the polymorphic form in the presence of at least one organic solvent and preferably at least one organic anti-solvent.

With regard to conceivable and preferred solvent and anti-solvents, reference is made to the respective disclosure hereinabove and to the respective embodiments 83 and 84 in the embodiment section hereinbelow.

Therefore, in case the polymorphic form comprised in the tablet is polymorphic form 6 of sofosbuvir, providing the polymorphic form in (a) preferably comprises
(i) providing sofosbuvir according to formula (I) in crystalline form, pseudo-crystalline form, amorphous form, or as a mixture of two or more of these forms;
(ii) preparing a mixture comprising the sofosbuvir provided in (i) and at least one organic solvent, wherein (ii) preferably either comprises
   (ii.1) preparing a solution of the sofosbuvir provided in (i) in at least one organic solvent, preferably in the at least one organic solvent as defined herein; (ii.2) adding at least one organic anti-solvent to the solution obtained in (ii.1), preferably the at least one organic anti-solvent as defined herein;
   or comprises suspending the sofosbuvir provided in (i) in a mixture of the at least one organic solvent, preferably the at least one organic solvent as defined herein, and at least one organic anti-solvent, preferably the at least one organic anti-solvent as defined herein;
(iii) subjecting the mixture obtained in (ii) to crystallization conditions, obtaining polymorphic form 6 of sofosbuvir in its mother liquor;
(iv) separating the polymorphic form 6 of sofosbuvir from its mother liquor, preferably comprising
   (iv.1) subjecting the mother liquor containing the polymorphic form 6 of sofosbuvir to a solids separation process, obtaining the polymorphic form 6;
   (iv.2) optionally washing the polymorphic form 6 of sofosbuvir obtained from (iv.1);
   (iv.3) drying the polymorphic form 6 of sofosbuvir obtained from (iv.1), optionally from (iv.2).

Therefore, in case the polymorphic form comprised in the tablet is polymorphic form 7 of sofosbuvir, providing the polymorphic form in (a) preferably comprises
(i) providing sofosbuvir in crystalline form, pseudo-crystalline form, amorphous form, or as a mixture of two or more of these forms;
(ii) preparing seed crystals comprising the polymorphic form 7 of sofosbuvir by a method comprising
   (ii.1) providing sofosbuvir in crystalline form, pseudo-crystalline form, amorphous form, or as a mixture of two or more of these forms;
   (ii.2) providing seed crystals of polymorphic form 6 of crystalline sofosbuvir;
   (ii.3) preparing a solution of the sofosbuvir provided in (ii.1) in a C₂-C₁₀ alcohol or in a mixture of two or more thereof;
   (ii.4) subjecting the solution provided in (ii.3) to crystallization conditions, comprising seeding the solution with the seed crystals provided in (ii.2), wherein during crystallization, the solution is not stirred;
   (ii.5) separating at least a portion of the crystalline form 7 of sofosbuvir from its mother liquor;
(iii) preparing a solution of sofosbuvir provided in (i) in an organic solvent being a C₂-C₅ alcohol or in a mixture of two or more thereof, and in at least one organic anti-solvent, preferably the at least one organic anti-solvent as defined herein;
(iv) subjecting the solution provided in (iii) to crystallization conditions, comprising seeding the solution with the seed crystals prepared in (ii), wherein during crystallization, at least a portion of the seeded solution, preferably at least the portion of the seeded solution containing most of the seed crystals, more preferably at least the portion of the seeded solution containing most of the seed crystals and the polymorphic form 7 formed, in particular at least the bottom of the seeded solution containing most of the seed crystals, preferably essentially all seed crystals, and the polymorphic form 7 formed, obtaining the polymorphic form 7 of sofosbuvir in its mother liquor;
(v) separating the polymorphic form 7 of sofosbuvir from its mother liquor, comprising
   (v.1) subjecting the mother liquor comprising the polymorphic form 7 of sofosbuvir to a solids separation process, preferably filtration, obtaining at least a portion of the polymorphic form 7 of sofosbuvir;
   (v.2) optionally washing the polymorphic form 7 of sofosbuvir obtained in (v.1);
   (v.3) drying the polymorphic form 7 of sofosbuvir obtained in (v.1) or (v.2), optionally in (v.2).

Optionally, the process of the present invention further comprises a step (d) of coating the tablet obtained in (c) with at least one coating agent, preferably comprising a polyvinylalcohol and optionally comprising at least one taste-masking agent. Regarding step (d), reference is also made to the section “Coating agent” hereinabove.

The present invention also relates to a tablet which is obtainable or obtained by the process as described above.

### Preferred Uses

The tablet of the present invention is preferably used for the treatment of hepatitis C in a human. Due to the high drug load of the tablet compared to the tablet described in the prior art, and thus, the provision of smaller tablets having the same drug load compared to the tablet of the prior art, the tablet of the invention allows, for example, for an easier swallowing and thus significantly contributes to heighten the patient's convenience.

Further, the present invention also relates to the use of a polymorphic form of crystalline sofosbuvir according to formula (I)

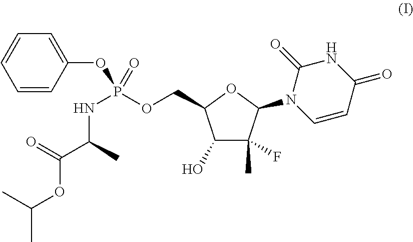 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

having a moisture stability of at least 95% for the preparation of tablet comprising the polymorphic form in an amount of at least 40 weight-%, based on the total weight of the tablet, wherein the moisture stability of the polymorphic form is defined as the amount of the polymorphic form which is present after an exposure to a relative humidity of (75±1) % at a temperature of (40±1)° C. for an exposure time of (18±1) hours, relative to the amount of the polymorphic form before said exposure, as determined via X-ray powder diffraction pattern analysis at a temperature in the range of from 15 to 25° C. with Cu-Kalpha_{1,2} radiation having a wavelength of 0.15419 nm before the exposure and after the exposure.

The present invention is further illustrated by the following embodiments and combinations of embodiments as indicated by the dependencies and references.
1. A tablet comprising a polymorphic form of crystalline sofosbuvir according to formula (I)

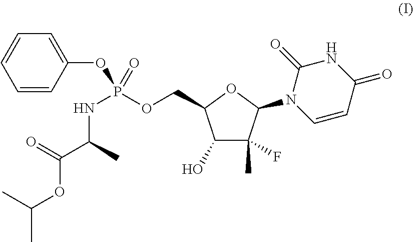 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

in an amount of at least 40 weight-%, based on the total weight of the tablet, wherein the polymorphic form has a moisture stability of at least 95%, wherein the moisture stability of the polymorphic form is defined as the amount of the polymorphic form which is present after an exposure to a relative humidity of (75±1) % at a temperature of (40±1)° C. for an exposure time of (18±1) hours, relative to the amount of the polymorphic form before said exposure, as determined via X-ray powder diffraction pattern analysis at a temperature in the range of from 15 to 25° C. with Cu-Kalpha_{1,2} radiation having a wavelength of 0.15419 nm before the exposure and after the exposure.
2. A tablet comprising a polymorphic form of crystalline sofosbuvir according to formula (I)

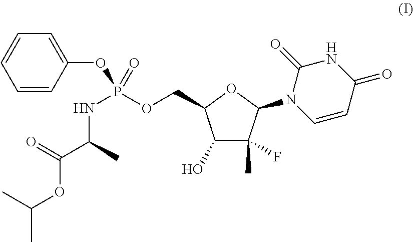 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

in an amount of at least 40 weight-%, preferably in an amount ≧75 weight-% based on the total weight of the tablet, wherein the polymorphic form has a moisture stability of at least 95%, wherein the moisture stability of the polymorphic form is defined as the amount of the polymorphic form which is present after an exposure to a relative humidity of (75±1) % at a temperature of (40±1)° C. for an exposure time of (18±1) hours, relative to the amount of the polymorphic form before said exposure, as determined via X-ray powder diffraction pattern analysis at a temperature in the range of from 15 to 25° C. with Cu-Kalpha_{1,2} radiation having a wavelength of 0.15419 nm before the exposure and after the exposure.
3. A tablet comprising a polymorphic form of crystalline sofosbuvir according to formula (I)

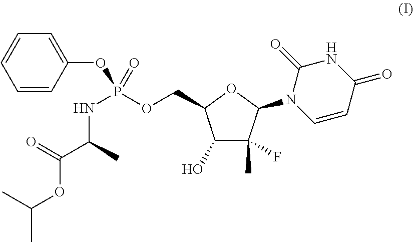 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

in an amount of at least 40 weight-%, preferably in an amount ≧75 weight-% based on the total weight of the tablet, wherein the polymorphic form has a moisture stability of at least 95%, wherein the moisture stability of the polymorphic form is defined as the amount of the polymorphic form which is present after an exposure to a relative humidity of (75±1) % at a temperature of (40±1)° C. for an exposure time of (18±1) hours, relative to the amount of the polymorphic form before said exposure, as determined via X-ray powder diffraction pattern analysis before the exposure and after the exposure and wherein the polymorphic form of crystalline sofosbuvir is polymorphic form 6 having an X-ray powder diffraction pattern with reflections at 2-theta values of (6.1±0.2)°, (8.2±0.2)°, (10.4±0.2)°, (12.7±0.2)°, (17.2±0.2)°, (17.7±0.2)°, (18.0±0.2)°, (18.8±±0.2)°, (19.4±0.2)°, (19.8±0.2)°, (20.1±0.2)°, (20.8±0.2)°, (21.8±0.2)°, (23.3±0.2)°, wherein the X-ray powder diffraction pattern are when measured at a temperature in the range of from 15 to 25° C. with Cu-Kalpha_{1,2} radiation having a wavelength of 0.15419 nm.
4. A tablet comprising a polymorphic form of crystalline sofosbuvir according to formula (I)

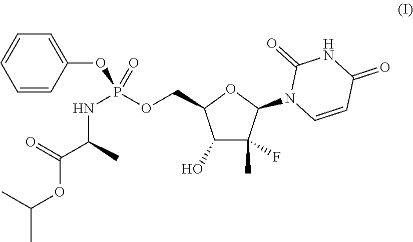 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

in an amount of at least 25 weight-%, or at least 35 weight-% or at least 40 weight-%, preferably in an amount ≧75 weight-% based on the total weight of the tablet, wherein the polymorphic form has a moisture stability of at least 95%, wherein the moisture stability of the polymorphic form is defined as the amount of the polymorphic form which is present after an exposure to a relative humidity of (75±1) % at a temperature of (40±1)° C. for an exposure time of (18±1) hours, relative to the amount of the polymorphic form before said exposure, as determined via X-ray powder diffraction pattern analysis before the exposure and after the exposure, wherein the polymorphic form is polymorphic form 7 having an X-ray powder diffraction pattern comprising no reflection at 2-theta angles in the range of from 2.0 to 7.8°, preferably comprising reflections at 2-theta values of (8.1±0.2)°, (10.4±0.2)°, (12.4±0.2)°, (17.3±0.2)°, (19.4±0.2)°, the X-ray powder diffraction pattern are when measured at a temperature in the range of from 15 to 25° C. with Cu-Kalpha_{1,2} radiation having a wavelength of 0.15419 nm.
5. The tablet of any of embodiments 1 to 4, wherein the polymorphic form is a combination of polymorphic form 6 and polymorphic form 7.
6. The tablet of any of embodiments 1 to 5, comprising the polymorphic form in an amount in the range of from 40 to 90 weight-%, preferably from 45 to 80 weight-%, based on the total weight of the tablet.
7. The tablet of any of embodiments 1 to 6, comprising the polymorphic form in an amount in the range of from 50 to 75 weight-%, based on the total weight of the tablet.
8. The tablet of any embodiments 1 to 7, comprising the polymorphic form in an amount of ≧75 weight-% based on the total weight of the tablet or in a range of from 75 to 90 weight-% or in a range of from 75 to 85 weight-%, or in a range of from 75 to 80 weight-% or in a range of from 80 to 90 weight-% or in a range of from 85 to 90 weight-%.
9. The tablet of any of embodiments 1 or 3, 5 to 8, comprising the polymorphic form in an amount of ≧75 weight-% based on the total weight of the tablet or in a range of from 75 to 90 weight-% or in a range of from 75 to 85 weight-%, or in a range of from 75 to 80 weight-% or in a range of from 80 to 90 weight-% or in a range of from 85 to 90 weight-% and wherein the polymorphic form is form 6.
10. The tablet of any of embodiments 1 or 2, 4 to 8, comprising the polymorphic form in an amount of at least ≧75 weight-% based on the total weight of the tablet or in a range of from 75 to 90 weight-% or in a range of from 75 to 85 weight-%, or in a range of from 75 to 80 weight-% or in a range of from 80 to 90 weight-% or in a range of from 85 to 90 weight-% and wherein the polymorphic form is form 7.
11. The tablet of any of embodiments 1 to 10, comprising the polymorphic form in an amount of ≧75 weight-% based on the total weight of the tablet or in a range of from 75 to 90 weight-% or in a range of from 75 to 85 weight-%, or in a range of from 75 to 80 weight-% or in a range of from 80 to 90 weight-% or in a range of from 85 to 90 weight-% and wherein the polymorphic form is a combination of form 6 and form 7.
12. The tablet of any of embodiments 1 to 11, further comprising at least one pharmaceutically acceptable excipient.
13. The tablet of embodiment 12, wherein at least 95 weight-%, preferably at least 97 weight-%, more preferably at least 99 weight-%, more preferably at least 99.5 weight-% of the tablet consist of the polymorphic form of crystalline sofosbuvir according to formula (I) and the at least one pharmaceutically acceptable excipient.
14. The tablet of embodiment 12 or 13, wherein at least 95 weight-%, preferably at least 97 weight-%, more preferably at least 99 weight-%, more preferably at least 99.5 weight-% of the tablet consist of the polymorphic form of crystalline sofosbuvir according to formula (I) and the at least one pharmaceutically acceptable excipient wherein the polymorphic form is form 6.
15. The tablet of embodiment 12 or 13, wherein at least 95 weight-%, preferably at least 97 weight-%, more preferably at least 99 weight-%, more preferably at least 99.5 weight-% of the tablet consist of the polymorphic form of crystalline sofosbuvir according to formula (I) and the at least one pharmaceutically acceptable excipient wherein the polymorphic form is form 7.
16. The tablet of any of embodiments 12 to 15, wherein at least 95 weight-%, preferably at least 97 weight-%, more preferably at least 99 weight-%, more preferably at least 99.5 weight-% of the tablet consist of the polymorphic form of crystalline sofosbuvir according to formula (I) and the at least one pharmaceutically acceptable excipient wherein the polymorphic form is a combination of form 6 and form 7.
17. The tablet of any of embodiments 12 to 16, wherein at least 99.9 weight-% of the tablet consist of the polymorphic form of crystalline sofosbuvir according to formula (I) and the at least one pharmaceutically acceptable excipient.
18. The tablet of embodiment 17, wherein at least 99.9 weight-% of the tablet consist of the polymorphic form of crystalline sofosbuvir according to formula (I) and the at least one pharmaceutically acceptable excipient wherein the polymorphic form is form 6.
19. The tablet of embodiment 17, wherein at least 99.9 weight-% of the tablet consist of the polymorphic form of crystalline sofosbuvir according to formula (I) and the at least one pharmaceutically acceptable excipient wherein the polymorphic form is form 7.
20. The tablet of any of embodiments 17 to 19, wherein at least 99.9 weight-% of the tablet consist of the polymorphic form of crystalline sofosbuvir according to formula (I) and the at least one pharmaceutically acceptable excipient wherein the polymorphic form is a combination of form 6 and form 7.
21. The tablet of any of embodiments 1 to 20, wherein the polymorphic form has a moisture stability of at least 96%, preferably at least 97%, more preferably at least 98%.
22. The tablet of any of embodiments 1 to 21, wherein the polymorphic form has a moisture stability of at least 99%.
23. The tablet of any of embodiments 1 to 22, wherein the polymorphic form is obtainable or obtained by a process comprising crystallization of the polymorphic form in the presence of at least one organic solvent and preferably at least one organic anti-solvent.
24. The tablet of any of embodiments 1 to 23, comprising at least one organic solvent.
25. The tablet of embodiment 24, preferably the tablet of embodiment 7 insofar as embodiment 7 is dependent of embodiment 6, wherein the at least one organic solvent comprises, preferably is, the at least one organic solvent in the presence of which the polymorphic form is crystallized.
26. The tablet of embodiment 24 or 25, comprising the at least one organic solvent in an amount in the range of from 0.002 to 0.3 weight-%, preferably of from 0.005 to 0.2 weight-%, based on the total weight of the tablet.
27. The tablet of embodiment 26, comprising the at least one organic solvent in an amount in the range of from 0.01 to 0.1 weight-%, based on the total weight of the tablet.
28. The tablet of any of embodiments 23 to 27, wherein the at least one organic solvent comprises an aliphatic alcohol, preferably a C₂ alcohol, a C₃ alcohol, a C₄ alcohol, a C₅ alcohol, or a mixture of two or more thereof, more preferably a C₂ alcohol, a C₃ alcohol, a C₄ alcohol, or a mixture of two or more thereof, more preferably ethanol, n-propanol, n-butanol, or a mixture of two or more thereof
29. The tablet of embodiment 28, wherein the at least one organic solvent comprises, preferably consists of, ethanol or n-butanol.
30. The tablet of any of embodiments 1 to 29, preferably according to any of embodiments 23 to 29, more preferably according to any of embodiments 24 to 29, more preferably according to any of embodiments 25 to 29, comprising at least one organic anti-solvent, preferably the at least one organic anti-solvent in the presence of which the polymorphic form is preferably crystallized.
31. The tablet of embodiment 30, comprising the at least one organic anti-solvent in an amount in the range of from 0.0002 to 0.05 weight-%, preferably of from 0.0005 to 0.03 weight-%, based on the total weight of the tablet.
32. The tablet of embodiment 31, comprising the at least one organic anti-solvent in an amount in the range of from 0.001 to 0.01 weight-%, based on the total weight of the tablet.
33. The tablet of any of embodiments 23 to 31, preferably according to any of embodiments 30 to 32, wherein the at least one organic anti-solvent comprises an alkane, preferably a C₅ alkane, a C₆ alkane, a C₇ alkane, a C₈ alkane, or a mixture of two or more thereof, more preferably a C₇ alkane.
34. The tablet of embodiment 33, wherein the at least one organic anti-solvent comprises, preferably consists of, n-heptane.
35. A tablet comprising a polymorphic form of crystalline sofosbuvir according to formula (I)

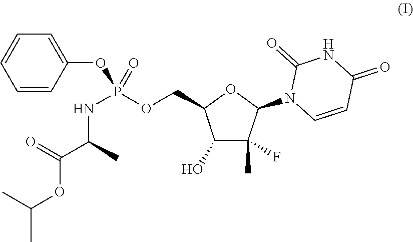 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

in an amount of at least 40 weight-%, preferably from 40 to 90 weight-%, more preferably from 45 to 80 weight-%, more preferably from 50 to 75 weight-%, based on the total weight of the tablet, wherein the polymorphic form is obtainable or obtained by a process comprising crystallization of the polymorphic form in the presence of at least one organic solvent and preferably at least one organic anti-solvent.
36. A tablet comprising a polymorphic form of crystalline sofosbuvir according to formula (I)

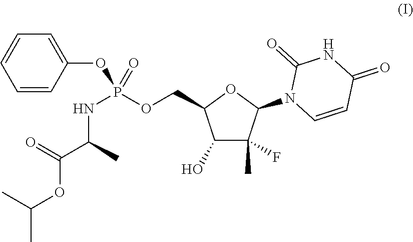 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

in an amount of at least 40 weight-%, preferably from 40 to 90 weight-%, more preferably from 45 to 80 weight-%, more preferably from 50 to 75 weight-%, based on the total weight of the tablet, wherein the polymorphic form is obtainable or obtained by a process comprising crystallization of the polymorphic form in the presence of at least one organic solvent and preferably at least one organic anti-solvent, wherein the polymorphic form of crystalline sofosbuvir is polymorphic form 6 having an X-ray powder diffraction pattern with reflections at 2-theta values of (6.1±0.2)°, (8.2±0.2)°, (10.4±0.2)°, (12.7±0.2)°, (17.2±0.2)°, (17.7±0.2)°, (18.0±0.2)°, (18.8±0.2)°, (19.4±0.2)°, (19.8±0.2)°, (20.1±0.2)°, (20.8±0.2)°, (21.8±0.2)°, (23.3±0.2)°, wherein the X-ray powder diffraction pattern are when measured at a temperature in the range of from 15 to 25° C. with Cu-Kalpha_{1,2} radiation having a wavelength of 0.15419 nm.
37. A tablet comprising a polymorphic form of crystalline sofosbuvir according to formula (I)

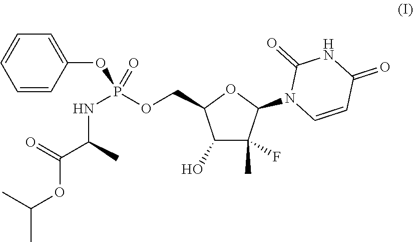 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

in an amount of at least 25 weight-%, or at least 35 weight-% or at least 40 weight-%, preferably from 40 to 90 weight-%, more preferably from 45 to 80 weight-%, more preferably from 50 to 75 weight-% based on the total weight of the tablet, wherein the polymorphic form is obtainable or obtained by a process comprising crystallization of the polymorphic form in the presence of at least one organic solvent and preferably at least one organic anti-solvent, wherein the polymorphic form is polymorphic form 7 having an X-ray powder diffraction pattern comprising no reflection at 2-theta angles in the range of from 2.0 to 7.8°, preferably comprising reflections at 2-theta values of (8.1±0.2)°, (10.4±0.2)°, (12.4±0.2)°, (17.3±0.2)°, (19.4±0.2)°, the X-ray powder diffraction pattern are when measured at a temperature in the range of from 15 to 25° C. with Cu-Kalpha_{1,2} radiation having a wavelength of 0.15419 nm.
38. The tablet of any of embodiments 35 to 37 comprising the polymorphic form in an amount of ≧75 weight-% based on the total weight of the tablet or in a range of from 75 to 90 weight-% or in a range of from 75 to 85 weight-%, or in a range of from 75 to 80 weight-% or in a range of from 80 to 90 weight-% or in a range of from 85 to 90 weight-%.
39. The tablet of any of embodiments 35 to 38, comprising the polymorphic form in an amount of ≧75 weight-% based on the total weight of the tablet or in a range of from 75 to 90 weight-% or in a range of from 75 to 85 weight-%, or in a range of from 75 to 80 weight-% or in a range of from 80 to 90 weight-% or in a range of from 85 to 90 weight-% and wherein the polymorphic form is form 6.
40. The tablet of any of embodiments 35 to 38, comprising the polymorphic form in an amount of at least ≧75 weight-% based on the total weight of the tablet or in a range of from 75 to 90 weight-% or in a range of from 75 to 85 weight-%, or in a range of from 75 to 80 weight-% or in a range of from 80 to 90 weight-% or in a range of from 85 to 90 weight-% and wherein the polymorphic form is form 7.
41. The tablet of any of embodiments 35 to 40, further comprising at least one pharmaceutically acceptable excipient.
42. The tablet of embodiment 41, wherein at least 95 weight-%, preferably at least 97 weight-%, more preferably at least 99 weight-%, more preferably at least 99.5 weight-% of the tablet consist of the polymorphic form of crystalline sofosbuvir according to formula (I) and the at least one pharmaceutically acceptable excipient.
43. The tablet of embodiment 41 or 42, wherein at least 95 weight-%, preferably at least 97 weight-%, more preferably at least 99 weight-%, more preferably at least 99.5 weight-% of the tablet consist of the polymorphic form of crystalline sofosbuvir according to formula (I) and the at least one pharmaceutically acceptable excipient wherein the polymorphic form is form 6.
44. The tablet of embodiment 41 or 42, wherein at least 95 weight-%, preferably at least 97 weight-%, more preferably at least 99 weight-%, more preferably at least 99.5 weight-% of the tablet consist of the polymorphic form of crystalline sofosbuvir according to formula (I) and the at least one pharmaceutically acceptable excipient wherein the polymorphic form is form 7.
45. The tablet of any of embodiments 41 to 44, wherein the polymorphic form is a combination of polymorphic form 6 and polymorphic form 7.
46. The tablet of any embodiment 41 to 45, wherein at least 99.9 weight-% of the tablet consist of the polymorphic form of crystalline sofosbuvir according to formula (I) and the at least one pharmaceutically acceptable excipient.
47. The tablet of any of embodiments 41 to 43, 45 to 46, wherein at least 99.9 weight-% of the tablet consist of the polymorphic form of crystalline sofosbuvir according to formula (I) and the at least one pharmaceutically acceptable excipient wherein the polymorphic form is form 6.
48. The tablet of any of embodiments 41 to 42, 44 to 46, wherein at least 99.9 weight-% of the tablet consist of the polymorphic form of crystalline sofosbuvir according to formula (I) and the at least one pharmaceutically acceptable excipient wherein the polymorphic form is form 7.
49. The tablet of any of embodiments 46 to 48, wherein the polymorphic form is a combination of polymorphic form 6 and polymorphic form 7.
50. The tablet of any of embodiments 35 to 49, comprising at least one organic solvent.
51. The tablet of embodiment 50, wherein the at least one organic solvent comprised in the tablet comprises, preferably is, the at least one organic solvent in the presence of which the polymorphic form is crystallized.
52. The tablet of embodiment 50 or 51, comprising the at least one organic solvent in an amount in the range of from 0.002 to 0.3 weight-%, preferably of from 0.005 to 0.2 weight-%, more preferably from 0.01 to 0.1 weight-%, based on the total weight of the tablet.
53. The tablet of any of embodiments 35 to 52, wherein the at least one organic solvent comprises an aliphatic alcohol, preferably a C₂ alcohol, a C₃ alcohol, a C₄ alcohol, a C₅ alcohol, or a mixture of two or more thereof, more preferably a C₂ alcohol, a C₃ alcohol, a C₄ alcohol, or a mixture of two or more thereof, more preferably ethanol, n-propanol, n-butanol, or a mixture of two or more thereof, wherein more preferably, the at least one organic solvent comprises, preferably consists of, ethanol or n-butanol.
54. The tablet of any of embodiments 35 to 53, preferably according to any of embodiments 19 to 22, more preferably according to any of embodiments 20 to 22, comprising at least one organic anti-solvent, preferably the at least one organic anti-solvent in the presence of which the polymorphic form is preferably crystallized.
55. The tablet of embodiment 54 comprising the at least one organic anti-solvent in an amount in the range of from 0.0002 to 0.05 weight-%, preferably of from 0.0005 to 0.03 weight-%, more preferably from 0.001 to 0.01 weight-%, based on the total weight of the tablet.
56. The tablet of any of embodiments 35 to 55, preferably according to any of embodiments 23 to 25, wherein the at least one organic anti-solvent comprises an alkane, preferably a C₅ alkane, a C₆ alkane, a C₇ alkane, a C₈ alkane, or a mixture of two or more thereof, more preferably a C₇ alkane, wherein more preferably, the at least one organic anti-solvent comprises, preferably consists of, n-heptane.
57. A tablet comprising a polymorphic form of crystalline sofosbuvir according to formula (I)

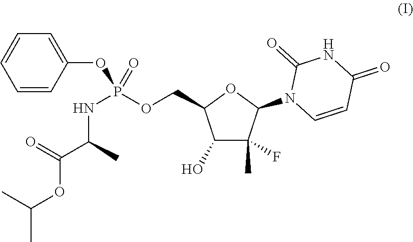 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

in an amount of at least 40 weight-%, preferably from 40 to 90 weight-%, more preferably from 45 to 80 weight-%, more preferably from 50 to 75 weight-%, preferably in an amount ≧75 weight-% based on the total weight of the tablet, comprising at least one organic solvent.
58. A tablet comprising a polymorphic form of crystalline sofosbuvir according to formula (I)

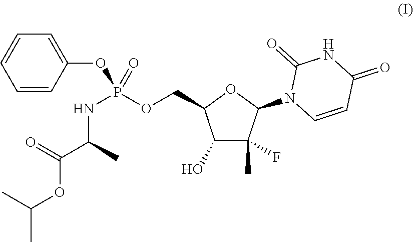 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

in an amount of at least 40 weight-%, preferably from 40 to 90 weight-%, more preferably from 45 to 80 weight-%, more preferably from 50 to 75 weight-%, preferably in an amount ≧75 weight-% based on the total weight of the tablet, comprising at least one organic solvent and wherein the polymorphic form of crystalline sofosbuvir is polymorphic form 6 having an X-ray powder diffraction pattern with reflections at 2-theta values of (6.1±0.2)°, (8.2±0.2)°, (10.4±0.2)°, (12.7±0.2)°, (17.2±0.2)°, (17.7±0.2)°, (18.0±0.2)°, (18.8±0.2)°, (19.4±0.2)°, (19.8±0.2)°, (20.1±0.2)°, (20.8±0.2)°, (21.8±0.2)°, (23.3±0.2)°, wherein the X-ray powder diffraction pattern are when measured at a temperature in the range of from 15 to 25° C. with Cu-Kalpha_{1,2} radiation having a wavelength of 0.15419 nm.
59. A tablet comprising a polymorphic form of crystalline sofosbuvir according to formula (I)

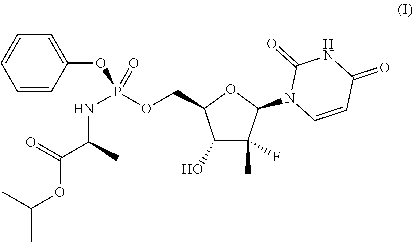 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

in an amount of at least 25 weight-%, of at least 30 weight-%, of at least 35 weight-% of at least 40 weight-%, preferably from 40 to 90 weight-%, more preferably from 45 to 80 weight-%, more preferably from 50 to 75 weight-%, preferably in an amount ≧75 weight-% based on the total weight of the tablet, comprising at least one organic solvent wherein the polymorphic form is polymorphic form 7 having an X-ray powder diffraction pattern comprising no reflection at 2-theta angles in the range of from 2.0 to 7.8°, preferably comprising reflections at 2-theta values of (8.1±0.2)°, (10.4±0.2)°, (12.4±0.2)°, (17.3±0.2)°, (19.4±0.2)°, the X-ray powder diffraction pattern are when measured at a temperature in the range of from 15 to 25° C. with Cu-Kalpha_{1,2} radiation having a wavelength of 0.15419 nm.
60. The tablet of any of embodiments 57 to 59, comprising the polymorphic form in an amount of ≧75 weight-% based on the total weight of the tablet or in a range of from 75 to 90 weight-% or in a range of from 75 to 85 weight-%, or in a range of from 75 to 80 weight-% or in a range of from 80 to 90 weight-% or in a range of from 85 to 90 weight-% and wherein the polymorphic form is form 6.
61. The tablet of any of embodiments 57 to 59, comprising the polymorphic form in an amount of at least ≧75 weight-% based on the total weight of the tablet or in a range of from 75 to 90 weight-% or in a range of from 75 to 85 weight-%, or in a range of from 75 to 80 weight-% or in a range of from 80 to 90 weight-% or in a range of from 85 to 90 weight-% and wherein the polymorphic form is form 7.
62. The tablet of any of embodiments 57 to 61, further comprising at least one pharmaceutically acceptable excipient.
63. The tablet of embodiment 62, wherein at least 95 weight-%, preferably at least 97 weight-%, more preferably at least 99 weight-%, more preferably at least 99.5 weight-% of the tablet consist of the polymorphic form of crystalline sofosbuvir according to formula (I) and the at least one pharmaceutically acceptable excipient.
64. The tablet of embodiment 62 or 63 wherein at least 95 weight-%, preferably at least 97 weight-%, more preferably at least 99 weight-%, more preferably at least 99.5 weight-% of the tablet consist of the polymorphic form of crystalline sofosbuvir according to formula (I) and the at least one pharmaceutically acceptable excipient wherein the polymorphic form is form 6.
65. The tablet of embodiment 62 or 63 wherein at least 95 weight-%, preferably at least 97 weight-%, more preferably at least 99 weight-%, more preferably at least 99.5 weight-% of the tablet consist of the polymorphic form of crystalline sofosbuvir according to formula (I) and the at least one pharmaceutically acceptable excipient wherein the polymorphic form is form 7.
66. The tablet of any of embodiments 62 to 65, wherein at least 99.9 weight-% of the tablet consist of the polymorphic form of crystalline sofosbuvir according to formula (I) and the at least one pharmaceutically acceptable excipient.
67. The tablet of any of embodiments 62 to 64, 66, wherein at least 99.9 weight-% of the tablet consist of the polymorphic form of crystalline sofosbuvir according to formula (I) and the at least one pharmaceutically acceptable excipient wherein the polymorphic form is form 6.
68. The tablet of any of embodiments 62 to 63, 65 to 66, wherein at least 99.9 weight-% of the tablet consist of the polymorphic form of crystalline sofosbuvir according to formula (I) and the at least one pharmaceutically acceptable excipient wherein the polymorphic form is form 7.
69. The tablet of any of embodiments 57 to 68, wherein the polymorphic form is a combination of polymorphic form 6 and polymorphic form 7.
70. The tablet of any of embodiment 57 to 69, comprising the at least one organic solvent in an amount in the range of from 0.002 to 0.3 weight-%, preferably of from 0.005 to 0.2 weight-%, more preferably from 0.01 to 0.1 weight-%, based on the total weight of the tablet.
71. The tablet of any of embodiments 57 to 70, wherein the at least one organic solvent comprises an aliphatic alcohol, preferably a C₂ alcohol, a C₃ alcohol, a C₄ alcohol, a C₅ alcohol, or a mixture of two or more thereof, more preferably a C₂ alcohol, a C₃ alcohol, a C₄ alcohol, or a mixture of two or more thereof, more preferably ethanol, n-propanol, n-butanol, or a mixture of two or more thereof, wherein more preferably, the at least one organic solvent comprises, preferably consists of, ethanol or n-butanol.
72. The tablet of any of embodiments 57 to 71, comprising at least one organic anti-solvent.
73. The tablet of embodiment 72, comprising the at least one organic anti-solvent in an amount in the range of from 0.0002 to 0.05 weight-%, preferably of from 0.0005 to 0.03 weight-%, more preferably from 0.001 to 0.01 weight-%, based on the total weight of the tablet.
74. The tablet of any of embodiments 71 to 73, wherein the at least one organic anti-solvent comprises an alkane, preferably a C₅ alkane, a C₆ alkane, a C₇ alkane, a C₈ alkane, or a mixture of two or more thereof, more preferably a C₇ alkane, wherein more preferably, the at least one organic anti-solvent comprises, preferably consists of, n-heptane.
75. The tablet of any of embodiments 1 to 34 or any of embodiment 35 to 56 or any of embodiments 57 to 74, wherein the polymorphic form is polymorphic form 6 having an X-ray powder diffraction pattern with reflections at 2-theta values of (6.1±0.2)°, (8.2±0.2)°, (10.4±0.2)°, (12.7±0.2)°, (17.2±0.2)°, (17.7±0.2)°, (18.0±0.2)°, (18.8±0.2)°, (19.4±0.2)°, (19.8±0.2)°, (20.1±0.2)°, (20.8±0.2)°, (21.8±0.2)°, (23.3±0.2)°, when measured at a temperature in the range of from 15 to 25° C. with Cu-Kalpha_{1,2} radiation having a wavelength of 0.15419 nm.
76. The tablet of embodiment 75, wherein the polymorphic form 6 is obtainable or obtained by a process according to any of embodiments 1 to 38 as described hereinabove in the respective embodiment section, preferably by a process comprising
   (i) providing sofosbuvir according to formula (I) in crystalline form, pseudo-crystalline form, amorphous form, or as a mixture of two or more of these forms;
   (ii) preparing a mixture comprising the sofosbuvir provided in (i) and at least one organic solvent, wherein (ii) preferably either comprises
      (ii.1) preparing a solution of the sofosbuvir provided in (i) in at least one organic solvent, preferably in the at least one organic solvent as defined in embodiment 11 or 12 hereinabove;
      (ii.2) adding at least one organic anti-solvent to the solution obtained in (ii.1), preferably the at least one organic anti-solvent as defined in embodiment 16 or 17 hereinabove;
   or comprises suspending the sofosbuvir provided in (i) in a mixture of the at least one organic solvent, preferably the at least one organic solvent as defined in embodiment 11 or 12 hereinabove, and at least one organic anti-solvent, preferably the at least one organic anti-solvent as defined in embodiment 16 or 17 hereinabove;
   (iii) subjecting the mixture obtained in (ii) to crystallization conditions, obtaining polymorphic form 6 of sofosbuvir in its mother liquor;
   (iv) separating the polymorphic form 6 of sofosbuvir from its mother liquor, preferably comprising
      (iv.1) subjecting the mother liquor containing the polymorphic form 6 of sofosbuvir to a solids separation process, obtaining the polymorphic form 6;
      (iv.2) optionally washing the polymorphic form 6 of sofosbuvir obtained from (iv.1);
      (iv.3) drying the polymorphic form 6 of sofosbuvir obtained from (iv.1), optionally from (iv.2).
77. The tablet of any of embodiments 1 to 34 or any of embodiment 35 to 56 or any of embodiments 57 to 74, wherein the polymorphic form is polymorphic form 7 having an X-ray powder diffraction pattern comprising no reflection at 2-theta angles in the range of from 2.0 to 7.8°, preferably comprising reflections at 2-theta values of (8.1±0.2)°, (10.4±0.2)°, (12.4±0.2)°, (17.3±0.2)°, (19.4±0.2)°, when measured at a temperature in the range of from 15 to 25° C. with Cu-Kalpha_{1,2} radiation having a wavelength of 0.15419 nm.
78. The tablet of embodiment 77, wherein the polymorphic form 7 is obtainable or obtained by a process according to any of embodiments 8 to 62 as described hereinabove in the respective embodiment section, preferably by a process comprising
   (i) providing sofosbuvir in crystalline form, pseudo-crystalline form, amorphous form, or as a mixture of two or more of these forms;
   (ii) preparing seed crystals comprising the polymorphic form 7 of sofosbuvir by a method comprising
      (ii.1) providing sofosbuvir in crystalline form, pseudo-crystalline form, amorphous form, or as a mixture of two or more of these forms;
      (ii.2) providing seed crystals of polymorphic form 6 of crystalline sofosbuvir, having an X-ray powder diffraction pattern with reflections at 2-theta values of (6.1±0.2)°, (8.2±0.2)°, (10.4±0.2)°, (12.7±0.2)°, (20.8±0.2)°, when measured at a temperature in the range of from 15 to 25° C. with Cu-Kalpha_{1,2} radiation having a wavelength of 0.15419 nm;
      (ii.3) preparing a solution of the sofosbuvir provided in (ii.1) in a C₂-C₁₀ alcohol or in a mixture of two or more thereof;
      (ii.4) subjecting the solution provided in (ii.3) to crystallization conditions, comprising seeding the solution with the seed crystals provided in (ii.2), wherein during crystallization, the solution is not stirred;
      (ii.5) separating at least a portion of the crystalline form 7 of sofosbuvir from its mother liquor;
   (iii) preparing a solution of sofosbuvir provided in (i) in an organic solvent being a C₂-C₅ alcohol or in a mixture of two or more thereof, and in at least one organic anti-solvent, preferably the at least one organic anti-solvent as defined in embodiment 16 or 17 hereinabove;
   (iv) subjecting the solution provided in (iii) to crystallization conditions, comprising seeding the solution with the seed crystals prepared in (ii), wherein during crystallization, at least a portion of the seeded solution, preferably at least the portion of the seeded solution containing most of the seed crystals, more preferably at least the portion of the seeded solution containing most of the seed crystals and the polymorphic form 7 formed, in particular at least the bottom of the seeded solution containing most of the seed crystals, preferably essentially all seed crystals, and the polymorphic form 7 formed, obtaining the polymorphic form 7 of sofosbuvir in its mother liquor;
   (v) separating the polymorphic form 7 of sofosbuvir from its mother liquor, comprising
      (v.1) subjecting the mother liquor comprising the polymorphic form 7 of sofosbuvir to a solids separation process, preferably filtration, obtaining at least a portion of the polymorphic form 7 of sofosbuvir;
      (v.2) optionally washing the polymorphic form 7 of sofosbuvir obtained in (v.1);
      (v.3) drying the polymorphic form 7 of sofosbuvir obtained in (v.1) or (v.2), optionally in (v.2).
79. The tablet of any of embodiments 1 to 34 or any of embodiments 35 to 56 or any of embodiments 57 to 74, wherein the polymorphic form is a mixture of polymorphic form 6 and polymorphic form 7, the polymorphic form 6 having an X-ray powder diffraction pattern with reflections at 2-theta values of (6.1±0.2)°, (8.2±0.2)°, (10.4±0.2)°, (12.7±0.2)°, (17.2±0.2)°, (17.7±0.2)°, (18.0±0.2)°, (18.8±0.2)°, (19.4±0.2)°, (19.8±0.2)°, (20.1±0.2)°, (20.8±0.2)°, (21.8±0.2)°, (23.3±0.2)°, when measured at a temperature in the range of from 15 to 25° C. with Cu-Kalpha_{1,2} radiation having a wavelength of 0.15419 nm, and the polymorphic form 7 having an X-ray powder diffraction pattern comprising no reflection at 2-theta angles in the range of from 2.0 to 7.8°, preferably comprising reflections at 2-theta values of (8.1±0.2)°, (10.4±0.2)°, (12.4±0.2)°, (17.3±0.2)°, (19.4±0.2)°, when measured at a temperature in the range of from 15 to 25° C. with Cu-Kalpha_{1,2} radiation having a wavelength of 0.15419 nm, wherein the polymorphic form 6 is preferably obtainable or obtained by a process as defined in embodiment 33 and/or wherein the polymorphic form 7 is preferably obtainable or obtained by a process as defined in embodiment 35.
80. The tablet of any of embodiments 1 to 79, further comprising at least one pharmaceutically acceptable excipient.
81. The tablet of embodiment 80, wherein the at least one pharmaceutically acceptable excipient comprises at least one diluent, or at least one disintegrant, or at least one glidant, or at least one lubricant, or a combination of at least one diluent and at least one disintegrant, or a combination of at least one diluent and at least one glidant, or a combination of at least one disintegrant and at least one lubricant, or a combination of at least one diluent and at least one disintegrant and at least one glidant, or a combination of at least one diluent and at least one disintegrant and at least one lubricant, or a combination of at least one disintegrant and at least one glidant and at least one lubricant, or a combination of at least one diluent and at least one disintegrant and at least one glidant and at least one lubricant, wherein the at least one pharmaceutically acceptable excipient preferably comprises a combination of at least one diluent and at least one disintegrant and at least one glidant and at least one lubricant.
82. The tablet of embodiment 81, wherein the at least one diluent comprises, preferably is, at least one of calcium carbonate, dicalcium phosphate, dry starch, calcium sulfate, cellulose, compressible sugars, confectioner's sugar, dextrates, dextrin, dextrose, dibasic calcium phosphate dihydrate, glyceryl palmitostearate, hydrogenated vegetable oil type I, inositol, kaolin, lactose, magnesium carbonate, magnesium oxide, maltodextrin, mannitol, microcrystalline cellulose, polymethacrylates, potassium chloride, powdered cellulose, powdered sugar, pregelatinized starch, sodium chloride, sorbitol, starch, sucrose, sugar spheres, talc, tribasic calcium phosphate, preferably at least one of dicalcium phosphate, cellulose, compressible sugars, dibasic calcium phosphate dehydrate, lactose, mannitol, microcrystalline cellulose, starch, tribasic calcium phosphate, more preferably at least one of mannitol, microcrystalline cellulose.
83. The tablet of embodiment 82, wherein the at least one diluent comprises, preferably is, a combination of mannitol and microcrystalline cellulose.
84. The tablet of any of embodiments 81 to 83, wherein the at least disintegrant comprises, preferably is, at least one of agar, alginic acid, bentonite, carboxymethylcellulose calcium, carboxymethylcellulose sodium, carboxymethylcellulose, cellulose, a cation exchange resin, cellulose, gums, citrus pulp, colloidal silicon dioxide, corn starch, croscarmellose sodium, crospovidone, guar gum, hydrous aluminum silicate, an ion exchange resin (e.g., polyacrin potassium), magnesium aluminum silicate, methyl cellulose, microcrystalline cellulose, modified cellulose gum, modified corn starch, montmorillonite clay, natural sponge, polyacrilin potassium, potato starch, powdered cellulose, povidone, pregelatinized starch, sodium alginate, sodium bicarbonate in admixture with an acidulant such as tartaric acid or citric acid, sodium starch glycolate, starch, silicates, preferably at least one of croscarmellose sodium, crospovidone, microcrystalline cellulose, modified corn starch, povidone, pregelatinized starch, sodium starch glycolate.
85. The tablet of embodiment 84, wherein the at least one disintegrant comprises, preferably is, croscarmellose sodium.
86. The tablet of any of embodiments 79 to 85, wherein the at least one glidant comprises, preferably is, is at least one of colloidal silicon dioxide, talc, starch, starch derivatives.
87. The tablet of embodiment 86, wherein the at least one glidant comprises, preferably is, colloidal silicon dioxide.
88. The tablet of any of embodiments 79 to 87, wherein the at least one lubricant comprises, preferably is, at least one of calcium stearate, glyceryl monostearate, glyceryl palmitostearate, hydrogenated castor oil, hydrogenated vegetable oil, light mineral oil, magnesium stearate, mineral oil, polyethylene glycol, sodium benzoate, sodium lauryl sulfate, sodium stearyl fumarate, stearic acid, talc, zinc stearate, preferably at least one of calcium stearate, magnesium stearate, polyethylene glycol, sodium stearyl fumarate, stearic acid, talc.
89. The tablet of embodiment 88, wherein the at least one lubricant comprises, preferably is, magnesium stearate.
90. The tablet of any of embodiments 79 to 89, wherein the at least one pharmaceutically acceptable excipient comprises mannitol, microcrystalline cellulose, croscarmellose sodium, colloidal silicon dioxide, and magnesium stearate.
91. The tablet of any of embodiments 79 to 90, wherein the at least one pharmaceutically acceptable excipient is a combination of mannitol, microcrystalline cellulose, croscarmellose sodium, colloidal silicon dioxide, and magnesium stearate.
92. The tablet of any of embodiments 79 to 91, wherein the at least one pharmaceutically acceptable excipient further comprises at least one coating agent.
93. The tablet of embodiment 92, wherein the at least one coating agent comprises, preferably is, at least one of hydroxypropylmethylcellulose, hydroxypropylcellulose, methylcellulose, ethylcellulose, hydroxyethylcellulose, cellulose acetate phthalate, sodium ethyl cellulose sulfate, carboxymethyl cellulose, polyvinylpyrolidone, zein, an acrylic polymer including methacrylic acid or methacrylic acid ester copolymers including methacrylic acid or methylmethacrylate copolymers, a polyvinyl alcohol.
94. The tablet of embodiment 93, wherein the at least one coating agent comprises, preferably is, a polyvinyl alcohol.
95. The tablet of a embodiment 93 or 94, wherein the at least one coating agent comprises at least one taste-masking agent.
96. The tablet of embodiment 95, wherein the at least one coating agent comprises, preferably is, a combination of a polyvinyl alcohol and at least one taste-masking agent.
97. The tablet of any of embodiments 80 to 96, wherein at least 95 weight-%, preferably at least 97 weight-%, more preferably at least 99 weight-%, more preferably at least 99.5 weight-% of the tablet consist of the polymorphic form of crystalline sofosbuvir according to formula (I) and the at least one pharmaceutically acceptable excipient.
98. The tablet of embodiment 97, wherein at least 99.9 weight-% of the tablet consist of the polymorphic form of crystalline sofosbuvir according to formula (I) and the at least one pharmaceutically acceptable excipient.
99. The tablet of any of embodiments 80 to 98, wherein the tablet contains the at least one pharmaceutically acceptable excipient in an amount of at most 60 weight-%, preferably in the range of from 60 to 10 weight-%, more preferably from 55 to 20 weight-%, more preferably from 50 to 30 weight-%, based on the total weight of the tablet.
100. The tablet of any of embodiments 80 to 99, wherein the tablet contains the at least one pharmaceutically acceptable excipient in an amount of <25 weight-% based on the total weight of the tablet or in a range of from 25 to 10 weight-% or in a range of from 25 to 15 weight-%, or in a range of from 25 to 20 weight-% or in a range of from 20 to 10 weight-% or in a range of from 15 to 10 weight-% based on the total weight of the tablet
101. The tablet of any of embodiments 80 to 100, wherein at least 95 weight-%, preferably at least 97 weight-%, more preferably at least 99 weight-%, more preferably at least 99.5 weight-% of the tablet consist of the polymorphic form of crystalline sofosbuvir according to formula (I), mannitol, microcrystalline cellulose, croscarmellose sodium, colloidal silicon dioxide, and magnesium stearate.
102. The tablet of embodiment 101, wherein at least 99.9 weight-% of the tablet consist of the polymorphic form of crystalline sofosbuvir according to formula (I), mannitol, microcrystalline cellulose, croscarmellose sodium, colloidal silicon dioxide, and magnesium stearate.
103. The tablet of any of embodiments 80 to 102, wherein the tablet contains mannitol, microcrystalline cellulose, croscarmellose sodium, colloidal silicon dioxide, and magnesium stearate in an amount of at most 60 weight-%, preferably in the range of from 60 to 10 weight-%, more preferably from 55 to 20 weight-%, more preferably from 50 to 30 weight-%, based on the total weight of the tablet.
104. The tablet of any of embodiments 80 to 103, wherein the tablet contains mannitol, microcrystalline cellulose, croscarmellose sodium, colloidal silicon dioxide, and magnesium stearate in an amount of <25 weight-% or in a range of from 25 to 10 weight-% or in a range of from 25 to 15 weight-%, or in a range of from 25 to 20 weight-% or in a range of from 20 to 10 weight-% or in a range of from 15 to 10 weight-% based on the total weight of the tablet.
105. The tablet of any of embodiments 80 to 104, wherein the at least one excipient comprises, preferably is, a combination of at least one diluent and at least one disintegrant and at least one glidant and at least one lubricant, and wherein the weight ratio of the at least one disintegrant relative to the at least diluent is in the range of from 0.01:1 to 0.5:1, preferably from 0.02:1 to 0.2:1, the weight ratio of the at least one glidant relative to the at least diluent is in the range of from 0.001:1 to 0.1:1, preferably from 0.005:1 to 0.05:1, and the weight ratio of the at least one lubricant relative to the at least diluent is in the range of from 0.005:1 to 0.2:1, preferably from 0.01:1 to 0.1:1.
106. The tablet of embodiment 105, wherein the weight ratio of the at least one disintegrant relative to the at least diluent is in the range of from 0.05:1 to 0.1:1, the weight ratio of the at least one glidant relative to the at least diluent is in the range of from 0.01:1 to 0.02:1, and the weight ratio of the at least one lubricant relative to the at least diluent is in the range of from 0.02:1 to 0.05:1.
107. The tablet of embodiment 105 or 106, wherein the at least one diluent comprises, preferably is, a combination of mannitol and microcrystalline cellulose, the at least one disintegrant comprises, preferably is, croscarmellose sodium, the at least one glidant comprises, preferably is, colloidal silicon dioxide, and the at least one lubricant comprises, preferably is, magnesium stearate.
108. The tablet of any of embodiments 1 to 107, having a mass in the range of from 0.45 to 1.00 g, preferably from 0.50 to 0.80 g.
109. The tablet of any of embodiments 1 to 108, preferably a round or oval tablet, having a longest dimension of at most 20 mm, preferably at most 18 mm.
110. The tablet of any of embodiments 1 to 109, exhibiting a dissolution of at least 40%, preferably at least 50% after 5 minutes in the mean dissolution profile as determined with a USP dissolution paddle apparatus 2 at 37° C., 75 r.p.m., using a phosphate buffer having a pH of 6.8 and a vessel having a volume of 900 ml.
111. The tablet of embodiment 110, exhibiting a dissolution of at least 60% after 5 minutes in the mean dissolution profile as determined with a USP dissolution paddle apparatus 2 at 37° C., 75 r.p.m., using a phosphate buffer having a pH of 6.8 and a vessel having a volume of 900 ml.
112. The tablet of any of embodiments 1 to 111, preferably of embodiment 110 or 111, exhibiting a dissolution of at least 60%, preferably at least 70% after 10 minutes in the mean dissolution profile as determined with a USP dissolution paddle apparatus 2 at 37° C., 75 r.p.m., using a phosphate buffer having a pH of 6.8 and a vessel having a volume of 900 ml.
113. The tablet of embodiment 112, exhibiting a dissolution of at least 80% after 10 minutes in the mean dissolution profile as determined with a USP dissolution paddle apparatus 2 at 37° C., 75 r.p.m., using a phosphate buffer having a pH of 6.8 and a vessel having a volume of 900 ml.
114. The tablet of any of embodiments 1 to 113, preferably of embodiment 112 or 113, exhibiting a dissolution of at least 80%, preferably at least 85% after 15 minutes in the mean dissolution profile as determined with a USP dissolution paddle apparatus 2 at 37° C., 75 r.p.m., using a phosphate buffer having a pH of 6.8 and a vessel having a volume of 900 ml.
115. The tablet of embodiment 114, exhibiting a dissolution of at least 90%, preferably at least 95% after 15 minutes in the mean dissolution profile as determined with a USP dissolution paddle apparatus 2 at 37° C., 75 r.p.m., using a phosphate buffer having a pH of 6.8 and a vessel having a volume of 900 ml.
116. The tablet of any of embodiments 1 to 115, wherein the moisture stability is defined as the amount in mg of the polymorphic form which is present after an exposure of an amount of 30 mg of the polymorphic form to a relative humidity of (75±1) % at a temperature of (40±1)° C. for an exposure time of (18±1) hours in a humidity chamber, relative to the amount of 30 mg of the polymorphic form before said exposure, as determined via X-ray powder diffraction pattern analysis at a temperature in the range of from 15 to 25° C. with Cu-Kalpha_{1,2} radiation having a wavelength of 0.15419 nm of the polymorphic form before the exposure and via said X-ray powder diffraction pattern analysis after the exposure.
117. A process for preparing a tablet according to any of embodiments 1 to 116, preferably a tablet according to any of embodiments 80 to 116, comprising at least one pharmaceutically acceptable excipient, said process comprising
   (a) providing the polymorphic form of crystalline sofosbuvir according to formula (I),

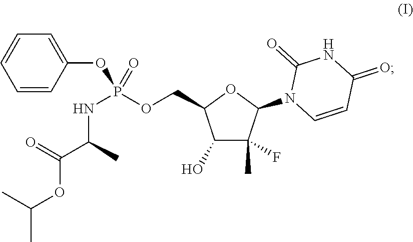 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

(b) blending the polymorphic form provided in (a) with at least one pharmaceutically acceptable excipient;
(c) preparing the tablet based on the blend obtained in (b).
118. A process for preparing a tablet according to any of embodiments 1 to 117, preferably a tablet according to any of embodiments 80 to 117, comprising at least one pharmaceutically acceptable excipient, said process comprising
   (a) providing the polymorphic form of crystalline sofosbuvir according to formula (I),

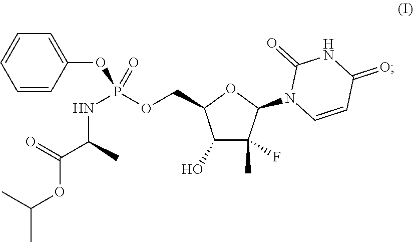 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

(b) blending the polymorphic form provided in (a) with at least one pharmaceutically acceptable excipient;
(c) preparing the tablet based on the blend obtained in (b), wherein the polymorphic form of crystalline sofosbuvir is polymorphic form 6 having an X-ray powder diffraction pattern with reflections at 2-theta values of (6.1±0.2)°, (8.2±0.2)°, (10.4±0.2)°, (12.7±0.2)°, (17.2±0.2)°, (17.7±0.2)°, (18.0±0.2)°, (18.8±0.2)°, (19.4±0.2)°, (19.8±0.2)°, (20.1±0.2)°, (20.8±0.2)°, (21.8±0.2)°, (23.3±0.2)°, wherein the X-ray powder diffraction pattern are when measured at a temperature in the range of from 15 to 25° C. with Cu-Kalpha_{1,2} radiation having a wavelength of 0.15419 nm.
119. A process for preparing a tablet according to any of embodiments 1 to 117, preferably a tablet according to any of embodiments 80 to 117, comprising at least one pharmaceutically acceptable excipient, said process comprising
   (a) providing the polymorphic form of crystalline sofosbuvir according to formula (I),

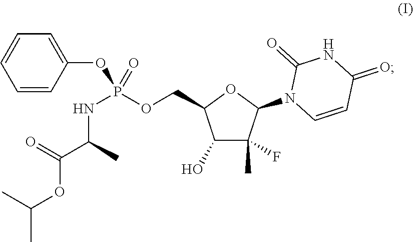 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

(b) blending the polymorphic form provided in (a) with at least one pharmaceutically acceptable excipient;
(c) preparing the tablet based on the blend obtained in (b) wherein the polymorphic form is polymorphic form 7 has an X-ray powder diffraction pattern comprising no reflection at 2-theta angles in the range of from 2.0 to 7.8°, preferably comprising reflections at 2-theta values of (8.1±0.2)°, (10.4±0.2)°, (12.4±0.2)°, (17.3±0.2)°, (19.4±0.2)°, the X-ray powder diffraction pattern are when measured at a temperature in the range of from 15 to 25° C. with Cu-Kalpha_{1,2} radiation having a wavelength of 0.15419 nm.
120. The process of any of embodiments 117 to 119, wherein the tablet of (c) comprises the polymorphic form in an amount ≧75 weight-%, preferably in the range of from 80 to 90 weight-%, more preferably in the range of from 85 to 90 weight-%, based on the total weight of the tablet.
121. The process of any of embodiments 117 to 120, wherein the tablet of (c) comprises the polymorphic form in an amount of ≧75 weight-% based on the total weight of the tablet or in a range of from 75 to 90 weight-% or in a range of from 75 to 85 weight-%, or in a range of from 75 to 80 weight-% or in a range of from 80 to 90 weight-% or in a range of from 85 to 90 weight-% and wherein the polymorphic form is form 6.
122. The process of any of embodiments 117 to 120, wherein the tablet of (c) comprises the polymorphic form in an amount of at least 25 weight-%, or at least 35 weight-% or at least 40 weight-% or at least ≧75 weight-% based on the total weight of the tablet or in a range of from 75 to 90 weight-% or in a range of from 75 to 85 weight-%, or in a range of from 75 to 80 weight-% or in a range of from 80 to 90 weight-% or in a range of from 85 to 90 weight-% and wherein the polymorphic form is form 7.
123. The process of any of embodiments 117 to 122 wherein the polymorphic form is a combination of polymorphic form 6 and polymorphic form 7.
124. The process of any of embodiments 117 to 123, wherein the at least one pharmaceutically acceptable excipient comprises, preferably is, a combination of at least one diluent and at least one disintegrant and at least one glidant and at least one lubricant.
125. The process of any of embodiment 117 to 124, wherein the at least one pharmaceutically acceptable excipient comprises, preferably is, a combination of mannitol, microcrystalline cellulose, croscarmellose sodium, colloidal silicon dioxide, and magnesium stearate.
126. The process of embodiment 124 or 125, wherein at least 95 weight-%, preferably at least 97 weight-%, more preferably at least 99 weight-%, more preferably at least 99.5 weight-% of the tablet of (c) consist of the polymorphic form of crystalline sofosbuvir according to formula (I) and the at least one pharmaceutically acceptable excipient.
127. The process of any of embodiments 124 to 126, wherein at least 95 weight-%, preferably at least 97 weight-%, more preferably at least 99 weight-%, more preferably at least 99.5 weight-% of the tablet of (c) consist of the polymorphic form of crystalline sofosbuvir according to formula (I) and the at least one pharmaceutically acceptable excipient wherein the polymorphic form is form 6.
128. The process of any of embodiments 124 to 126 wherein at least 95 weight-%, preferably at least 97 weight-%, more preferably at least 99 weight-%, more preferably at least 99.5 weight-% of the tablet of (c) consist of the polymorphic form of crystalline sofosbuvir according to formula (I) and the at least one pharmaceutically acceptable excipient wherein the polymorphic form is form 7.
129. The process of any of embodiments 124 to 128, wherein at least 99.9 weight-% of the tablet of (c) consist of the polymorphic form of crystalline sofosbuvir according to formula (I) and the at least one pharmaceutically acceptable excipient.
130. The process of any of embodiment 124 to 127, 129 wherein at least 99.9 weight-% of the tablet of (c) consist of the polymorphic form of crystalline sofosbuvir according to formula (I) and the at least one pharmaceutically acceptable excipient wherein the polymorphic form is form 6.
131. The process of any of embodiments 124 to 126, 128 to 129, wherein at least 99.9 weight-% of the tablet of (c) consist of the polymorphic form of crystalline sofosbuvir according to formula (I) and the at least one pharmaceutically acceptable excipient wherein the polymorphic form is form 7.
132. The process of any of embodiments 117 to 122 wherein the polymorphic form is a combination of polymorphic form 6 and polymorphic form 7.
133. The process of any of embodiments 124 to 132, wherein the blending according to (b) is carried out by wet granulation or by dry granulation, preferably by dry granulation.
134. The process of any of embodiments 117 to 133, wherein (b) comprises
   (b.1) blending the polymorphic form provided in (a) with a first portion of the at least one pharmaceutically acceptable excipient;
   (b.2) compressing the blend obtained in (b.1), obtaining a molding, preferably a tablet;
   (b.3) crushing the molding, preferably the tablet, obtained in (b.2), preferably over a sieve;
   (b.4) blending the particles obtained in (b.3) with a second portion of the at least one pharmaceutically acceptable excipient;
   and wherein (c) comprises
   (c) compressing the blend obtained in (b.4), obtaining the tablet.
135. The process of embodiment 134, wherein the total weight of the at least one pharmaceutically acceptable excipient contained in the first portion in (b.1) relative to the total weight of the at least one pharmaceutically acceptable excipient contained in the first portion in (b.1) and the second portion in (b.4) is in the range of from 50 to 95%, preferably from 70 to 90%.
136. The process of embodiment 134 or 135, the at least one pharmaceutically acceptable excipient comprising, preferably being, a combination of mannitol, microcrystalline cellulose, croscarmellose sodium, colloidal silicon dioxide, and magnesium stearate, wherein the first portion in (b.1) comprises, preferably is, a combination of mannitol, microcrystalline cellulose, croscarmellose sodium, colloidal silicon dioxide, and magnesium stearate, and wherein the second portion in (b.2) comprises, preferably is, a combination of microcrystalline cellulose, croscarmellose sodium, colloidal silicon dioxide, and magnesium stearate.
137. The process of any of embodiments 134 to 136, wherein the compressing in (b.2) is carried out under a pressure in the range of from 50 to 250 bar, preferably from 100 to 150 bar.
138. The process of any of embodiments 134 to 137, wherein in (b.3), the sieve has mesh size in the range of from 0.5 to 1.5 mm, preferably from 0.75 to 1.25 mm.
139. The process of any of embodiments 134 to 138, wherein the compressing in (c) is carried out under a pressure in the range of from 50 to 300 bar, preferably from 100 to 250 bar.
140. The process of any of embodiments 134 to 139, wherein providing the polymorphic form of crystalline sofosbuvir according to formula (I) in (a) comprises crystallizing the polymorphic form in the presence of at least one organic solvent and preferably at least one organic anti-solvent.
141. The process of embodiment 140, wherein the at least one organic solvent comprises an aliphatic alcohol, preferably a C₂ alcohol, a C₃ alcohol, a C₄ alcohol, a C₅ alcohol, or a mixture of two or more thereof, more preferably a C₂ alcohol, a C₃ alcohol, a C₄ alcohol, or a mixture of two or more thereof, more preferably ethanol, n-propanol, n-butanol, or a mixture of two or more thereof, wherein more preferably, the at least one organic solvent comprises, preferably consists of, ethanol or n-butanol.
142. The process of embodiment 140 or 141, wherein providing the polymorphic form of crystalline sofosbuvir according to formula (I) in (a) comprises crystallizing the polymorphic form in the presence of at least one organic solvent and at least one organic anti-solvent, wherein the at least one organic anti-solvent comprises an alkane, preferably a C₅ alkane, a C₆ alkane, a C₇ alkane, a C₈ alkane, or a mixture of two or more thereof, more preferably a C₇ alkane, wherein more preferably, the at least one organic anti-solvent comprises, preferably consists of, n-heptane.
143. The process of any of embodiments 140 to 142, wherein the polymorphic form of crystalline sofosbuvir is polymorphic form 6 having an X-ray powder diffraction pattern with reflections at 2-theta values of (6.1±0.2)°, (8.2±0.2)°, (10.4±0.2)°, (12.7±0.2)°, (17.2±0.2)°, (17.7±0.2)°, (18.0±0.2)°, (18.8±0.2)°, (19.4±0.2)°, (19.8±0.2)°, (20.1±0.2)°, (20.8±0.2)°, (21.8±0.2)°, (23.3±0.2)°, when measured at a temperature in the range of from 15 to 25° C. with Cu-Kalpha_{1,2} radiation having a wavelength of 0.15419 nm, wherein providing the polymorphic form in (a) comprises
   (i) providing sofosbuvir according to formula (I) in crystalline form, pseudo-crystalline form, amorphous form, or as a mixture of two or more of these forms;
   (ii) preparing a mixture comprising the sofosbuvir provided in (i) and at least one organic solvent, wherein (ii) preferably either comprises
      (ii.1) preparing a solution of the sofosbuvir provided in (i) in at least one organic solvent, preferably in the at least one organic solvent as defined in embodiment 11 or 12 hereinabove;
      (ii.2) adding at least one organic anti-solvent to the solution obtained in (ii.1), preferably the at least one organic anti-solvent as defined in embodiment 16 or 17 hereinabove;
   or comprises suspending the sofosbuvir provided in (i) in a mixture of the at least one organic solvent, preferably the at least one organic solvent as defined in embodiment 11 or 12 hereinabove, and at least one organic anti-solvent, preferably the at least one organic anti-solvent as defined in embodiment 16 or 17 hereinabove;
   (iii) subjecting the mixture obtained in (ii) to crystallization conditions, obtaining polymorphic form 6 of sofosbuvir in its mother liquor;
   (iv) separating the polymorphic form 6 of sofosbuvir from its mother liquor, preferably comprising
      (iv.1) subjecting the mother liquor containing the polymorphic form 6 of sofosbuvir to a solids separation process, obtaining the polymorphic form 6;
      (iv.2) optionally washing the polymorphic form 6 of sofosbuvir obtained from (iv.1);
      (iv.3) drying the polymorphic form 6 of sofosbuvir obtained from (iv.1), optionally from (iv.2).
144. The process of any of embodiments 140 to 142, wherein the polymorphic form of crystalline sofosbuvir is polymorphic form 7 having an X-ray powder diffraction pattern comprising no reflection at 2-theta angles in the range of from 2.0 to 7.8°, preferably comprising reflections at 2-theta values of (8.1±0.2)°, (10.4±0.2)°, (12.4±0.2)°, (17.3±0.2)°, (19.4±0.2)°, when measured at a temperature in the range of from 15 to 25° C. with Cu-Kalpha_{1,2} radiation having a wavelength of 0.15419 nm, wherein providing the polymorphic form in (a) comprises
   (i) providing sofosbuvir in crystalline form, pseudo-crystalline form, amorphous form, or as a mixture of two or more of these forms;
   (ii) preparing seed crystals comprising the polymorphic form 7 of sofosbuvir according by a method comprising
      (ii.1) providing sofosbuvir in crystalline form, pseudo-crystalline form, amorphous form, or as a mixture of two or more of these forms;
      (ii.2) providing seed crystals of polymorphic form 6 of crystalline sofosbuvir, having an X-ray powder diffraction pattern with reflections at 2-theta values of (6.1±0.2)°, (8.2±0.2)°, (10.4±0.2)°, (12.7±0.2)°, (20.8±0.2) °, when measured at a temperature in the range of from 15 to 25° C. with Cu-Kalpha_{1,2} radiation having a wavelength of 0.15419 nm;
      (ii.3) preparing a solution of the sofosbuvir provided in (ii.1) in a C₂-C₁₀ alcohol or in a mixture of two or more thereof;
      (ii.4) subjecting the solution provided in (ii.3) to crystallization conditions, comprising seeding the solution with the seed crystals provided in (ii.2), wherein during crystallization, the solution is not stirred;
      (ii.5) separating at least a portion of the crystalline form 7 of sofosbuvir from its mother liquor;
   (iii) preparing a solution of sofosbuvir provided in (i) in an organic solvent being a C₂-C₅ alcohol or in a mixture of two or more thereof, and in at least one organic anti-solvent, preferably the at least one organic anti-solvent as defined in embodiment 16 or 17 hereinabove;
   (iv) subjecting the solution provided in (iii) to crystallization conditions, comprising seeding the solution with the seed crystals prepared in (ii), wherein during crystallization, at least a portion of the seeded solution, preferably at least the portion of the seeded solution containing most of the seed crystals, more preferably at least the portion of the seeded solution containing most of the seed crystals and the polymorphic form 7 formed, in particular at least the bottom of the seeded solution containing most of the seed crystals, preferably essentially all seed crystals, and the polymorphic form 7 formed, obtaining the polymorphic form 7 of sofosbuvir in its mother liquor;
   (v) separating the polymorphic form 7 of sofosbuvir from its mother liquor, comprising
   (v.1) subjecting the mother liquor comprising the polymorphic form 7 of sofosbuvir to a solids separation process, preferably filtration, obtaining at least a portion of the polymorphic form 7 of sofosbuvir;
   (v.2) optionally washing the polymorphic form 7 of sofosbuvir obtained in (v.1);
   (v.3) drying the polymorphic form 7 of sofosbuvir obtained in (v.1), optionally in (v.2).
145. The process of any of embodiments 117 to 146, further comprising
   (d) coating the tablet obtained in (c) with at least one coating agent, preferably comprising a polyvinyl alcohol and optionally comprising at least one taste-masking agent.
146. A tablet, obtainable of obtained by the process according to any of embodiments 117 to 145.
147. A tablet according to any of embodiments 1 to 116 or according to embodiment 146 for for use in a method for treating hepatitis C in a human.
148. A tablet according to any of embodiments 1 to 116 or according to embodiment 146 for treating hepatitis C in a human.
149. Use of a tablet according to any of embodiments 1 to 116 or according to embodiment 146 for the preparation of a medicament for treating hepatitis C in a human.
150. A method for treating hepatitis C comprising administering a tablet according to any of embodiments 1 to 116 or according to embodiment 146 to a human in need thereof.
151. Use of a polymorphic form of crystalline sofosbuvir according to formula (I)

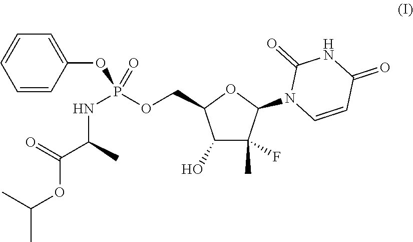 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

having a moisture stability of at least 95% for the preparation of tablet comprising the polymorphic form in an amount of at least 40 weight-%, preferably in an amount ≧75 weight-% based on the total weight of the tablet, wherein the moisture stability of the polymorphic form is defined as the amount of the polymorphic form which is present after an exposure to a relative humidity of (75±1) % at a temperature of (40±1)° C. for an exposure time of (18±1) hours, relative to the amount of the polymorphic form before said exposure, as determined via X-ray powder diffraction pattern analysis at a temperature in the range of from 15 to 25° C. with Cu-Kalpha_{1,2} radiation having a wavelength of 0.15419 nm before the exposure and after the exposure.
152. Use of a polymorphic form of crystalline sofosbuvir according to formula (I)

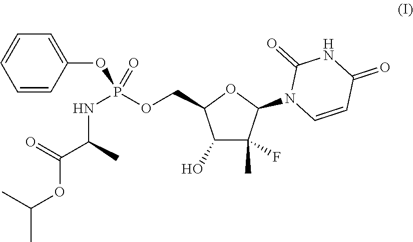 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

having a moisture stability of at least 95% for the preparation of tablet comprising the polymorphic form in an amount of at least 40 weight-%, preferably in an amount ≧75 weight-% based on the total weight of the tablet, wherein the moisture stability of the polymorphic form is defined as the amount of the polymorphic form which is present after an exposure to a relative humidity of (75±1) % at a temperature of (40±1)° C. for an exposure time of (18±1) hours, relative to the amount of the polymorphic form before said exposure, as determined via X-ray powder diffraction pattern analysis before the exposure and after the exposure wherein the polymorphic form of crystalline sofosbuvir is polymorphic form 6 having an X-ray powder diffraction pattern with reflections at 2-theta values of (6.1±0.2)°, (8.2±0.2)°, (10.4±0.2)°, (12.7±0.2)°, (17.2±0.2)°, (17.7±0.2)°, (18.0±0.2)°, (18.8±0.2)°, (19.4±0.2)°, (19.8±0.2)°, (20.1±0.2)°, (20.8±0.2)°, (21.8±0.2)°, (23.3±0.2)°, wherein the X-ray powder diffraction pattern are when measured at a temperature in the range of from 15 to 25° C. with Cu-Kalpha_{1,2} radiation having a wavelength of 0.15419 nm.
153. Use of a polymorphic form of crystalline sofosbuvir according to formula (I)

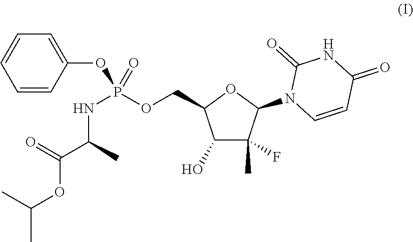 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

having a moisture stability of at least 95% for the preparation of tablet comprising the polymorphic form in an amount of at least 25% or of at least 30% or at least 35% or at least 40 weight-%, preferably in an amount ≧75 weight-% based on the total weight of the tablet, wherein the moisture stability of the polymorphic form is defined as the amount of the polymorphic form which is present after an exposure to a relative humidity of (75±1) % at a temperature of (40±1)° C. for an exposure time of (18±1) hours, relative to the amount of the polymorphic form before said exposure, as determined via X-ray powder diffraction pattern analysis at a temperature in the range of from 15 to 25° C. with Cu-Kalpha_{1,2} radiation having a wavelength of 0.15419 nm before the exposure and after the exposure wherein the polymorphic form is polymorphic form 7 having an X-ray powder diffraction pattern comprising no reflection at 2-theta angles in the range of from 2.0 to 7.8°, preferably comprising reflections at 2-theta values of (8.1±0.2)°, (10.4±0.2)°, (12.4±0.2)°, (17.3±0.2)°, (19.4±0.2)°, the X-ray powder diffraction pattern are when measured at a temperature in the range of from 15 to 25° C. with Cu-Kalpha_{1,2} radiation having a wavelength of 0.15419 nm.
154. The use of any of embodiments 151 to 153, wherein the tablet comprises the polymorphic form in an amount in the range of from 40 to 90 weight-%, preferably from 45 to 80 weight-%, more preferably from 50 to 75 weight-%, based on the total weight of the tablet.
155. The use of any of embodiments 151 to 154, wherein the tablet comprises the polymorphic form in an amount of ≧75 weight-% based on the total weight of the tablet or in a range of from 75 to 90 weight-% or in a range of from 75 to 85 weight-%, or in a range of from 75 to 80 weight-% or in a range of from 80 to 90 weight-% or in a range of from 85 to 90 weight-% based on the total weight of the tablet.
156. The use of any of embodiments 151 to 155, wherein the tablet further comprises at least one pharmaceutically acceptable excipient.
157. The use of embodiment 156 wherein at least 95 weight-%, preferably at least 97 weight-%, more preferably at least 99 weight-%, more preferably at least 99.5 weight-% of the tablet of (c) consist of the polymorphic form of crystalline sofosbuvir according to formula (I) and the at least one pharmaceutically acceptable excipient.
158. The use of embodiment 156 or 157, wherein at least 95 weight-%, preferably at least 97 weight-%, more preferably at least 99 weight-%, more preferably at least 99.5 weight-% of the tablet of (c) consist of the polymorphic form of crystalline sofosbuvir according to formula (I) and the at least one pharmaceutically acceptable excipient wherein the polymorphic form is form 6.
159. The use of embodiment 156 or 157, wherein at least 95 weight-%, preferably at least 97 weight-%, more preferably at least 99 weight-%, more preferably at least 99.5 weight-% of the tablet of (c) consist of the polymorphic form of crystalline sofosbuvir according to formula (I) and the at least one pharmaceutically acceptable excipient wherein the polymorphic form is form 7
160. The use of any embodiments 156 to 159, wherein at least 99.9 weight-% of the tablet of (c) consist of the polymorphic form of crystalline sofosbuvir according to formula (I) and the at least one pharmaceutically acceptable excipient.
161. The use of any embodiments 156 to 160, wherein at least 99.9 weight-% of the tablet of (c) consist of the polymorphic form of crystalline sofosbuvir according to formula (I) and the at least one pharmaceutically acceptable excipient wherein the polymorphic form is form 6.
162. The use of embodiment 156 to 160, wherein at least 99.9 weight-% of the tablet of (c) consist of the polymorphic form of crystalline sofosbuvir according to formula (I) and the at least one pharmaceutically acceptable excipient wherein the polymorphic form is form 7.
163. The use of embodiment 151 to 162, wherein the polymorphic form is a combination of polymorphic form 6 and polymorphic form 7.
164. The use of embodiment 151 to 163, wherein the polymorphic form has a moisture stability of at least 96%, preferably at least 97%, more preferably at least 98%, more preferably at least 99%.
165. The use of any of embodiments 151 to 164, wherein the tablet exhibits a dissolution of at least 40%, preferably at least 50%, more preferably at least 60%, after 5 minutes, at least 60%, preferably at least 70%, more preferably at least 80% after 10 minutes, and at least 80%, preferably at least 85%, more preferably at least 90%, more preferably at least 95%, after 15 minutes, in the mean dissolution profile as determined with a USP dissolution paddle apparatus 2 at 37° C., 75 r.p.m., using a phosphate buffer having a pH of 6.8 and a vessel having a volume of 900 ml.

The present invention is further illustrated by the following reference examples, examples, and comparative examples.

### Reference Example 1: Preparation of Amorphous Sofosbuvir

RE1.1 Amorphous sofosbuvir was prepared according to the following recipe: To 400 mg of sofosbuvir crystalline Form 1 prepared according to WO 2011/123645 A, Example 10, 3.5 mL ethanol were added, followed by 12 mL DI water. The mixture was subjected to sonication (2 to 5 minutes at room temperature in a VWR Ultrasonic Cleaner apparatus) to accelerate the dissolution of the solid material. The homogeneous solution was frozen in a bath of liquid nitrogen and lyophilized at −36° C. at a pressure of from 0 to 2 mbar, yielding amorphous sofosbuvir. It was characterized via XRPD analysis according to Reference Example 6. The respectively obtained XRPD pattern is shown in FIG. 1.
RE1.2 Amorphous sofosbuvir was prepared according to WO 2011/123645 A, Example 10, following by material dissolution in acetone (3 mL/g sofosbuvir) and solvent removal to dryness via evaporation at a temperature of 40° C. and a pressure of from 400 to 15 mbar. 5.00 g of this amorphous material was then suspended in 100 mL of heptane. The mixture was stirred at room temperature for 24 hours. The suspension was filtered on a medium-porosity glass fritted funnel and the solid residue dried at a temperature of 40° C. and a pressure of 50 mbar for 15 hours, yielding 3.95 g of amorphous sofosbuvir.

### Reference Example 2: Preparation of Polymorphic Form 1 of Sofosbuvir

RE2.1 Crystalline form 1 was prepared according to WO 2011/123645 A, Example 10-7. Work up and material purification were slightly modified: after 72 h at 5° C. the reaction mixture was cooled to a temperature of −5° C. and quenched by addition of satu-rated ammonium chloride and concentrated under vacuum to give brown foam. The residue was suspended in ethyl acetate, washed with water, NaHCO₃ and water. After concentration under vacuum, the crude product was purified by column chromatography using ethyl acetate/hexane mixtures (2:1 v/v to 1:0 v/v). The resulting white foam was dissolved in dichloromethane and stirred overnight. Crystalline product was collected by filtration and dried under vacuum.
RE2.2 Amorphous sofosbuvir was prepared according to WO 2011/123645 A, Example 10, following by material dissolution in acetone (3 mL/g sofosbuvir) and solvent removal to dryness via evaporation at a temperature of 40° C. and a pressure of from 400 to 15 mbar. 14.40 g of amorphous sofosbuvir were dissolved in 50 mL of dichloromethane at room temperature. After two minutes precipitation occurred and the mixture was diluted with additional 20 mL of dichloromethane. The suspension was stirred for 3 hours at room temperature, followed by slow addition of 50 mL of heptane. After complete heptane addition, the mixture was stirred at 0° C. for 1 hour and the suspension was filtered on a medium-porosity glass fitted funnel. The filter cake was washed with 10 mL of a 1:1 mixture of MTBE and dichloromethane and dried at a temperature of 40° C. and a pressure of 50 mbar for 12 hours, yielding 10.30 g of crystalline form 1 of sofosbuvir.
   It was characterized via XRPD analysis according to Reference Example 6. The respectively obtained XRPD pattern is shown in FIG. 3.

### Reference Example 3: Preparation of Polymorphic Form 6 of Sofosbuvir

In a vial equipped with a magnetic stirring bar were added 1.00 g of sofosbuvir (crystalline form 1 prepared according to WO 2011/123645 A, Example 10) and 2.5 mL of n-butanol (≧99.5%, Merck KGaA). The solution was heated to a temperature of from 50 to 60° C. to allow solid dissolution. 5.0 mL of n-heptane (≧99%, Sigma-Aldrich) were added under stirring conditions (500 r.p.m.) at a temperature of from 50 to 60° C. The solution was further stirred at a velocity of from 300 to 400 r.p.m. and a temperature of from 25 to 28° C. for 6 h to allow crystallization to take place. The obtained suspension was filtered on a medium-porosity glass fitted funnel and the solid residue was washed with n-heptane and dried under vacuum at a pressure of from 20 to 30 mbar and a temperature of 40° C. for 3 h whereafter heating was stopped and the temperature of the solid residue was allowed to decrease slowly to 25° C. at a pressure of from 20 to 30 mbar for 12 h, yielding 0.96 g (96% yield) of crystalline form 6 of sofosbuvir. It was characterized via XRPD analysis according to Reference Example 6. The respectively obtained XRPD pattern is shown in FIG. 5.

### Reference Example 4: Preparation of Polymorphic Form 7 of Sofosbuvir

RE4.1 In a first step, see crystals were prepared. Crystalline sofosbuvir of polymorphic form 1 (0.095 g, prepared according to WO 2011/123645 A1, example 10) was dissolved in 4 mL 2-octanol upon heating to a temperature in the range of from 50 to 60° C. The hot solution was filtered and allowed to cool to room temperature. Thereafter, sofosbuvir form 6 seed crystals (5 mg, prepared according to WO 2011/123645 A1, example 21) were added. The mixture was allowed to stand at room temperature without mechanical agitation and after one week, crystals deposited at the solution/air boundary. The crystals were collected and air dried. The XRPD pattern confirmed that sofosbuvir of the crystalline form 7 was obtained. These crystals were used as seed crystals for the following process:
   A 50 mL reactor equipped with a pitched-blade stirrer was charged with 1.00 g of sofosbuvir of crystalline form 1 (prepared according to WO 2011/123645 A1, Example 10) and 10 mL of n-butanol. The solution was heated to 40-45° C. under stirring (300 r.p.m.) to allow solid dissolution, followed by addition of 26 mL of n-heptane at 40-45° C. The solution was cooled to 25° C. After 10 minutes, the stirring was stopped and the pitched-blade stirrer placed on the upper part of the solution, followed by addition of seed of polymorphic form 7 (50 mg, prepared according to the process above in RE4.1). After 10 minutes, the upper part of the solution was slowly stirred (100 r.p.m.). No motion was applied to the lower part of the solution containing the seed crystals, allowing crystal formation on the bottom of the reactor. After 6 hours the reaction was stopped. The solvent was filtered out and the solid residue was collected, washed with n-heptane and dried under vacuum at a pressure of from 20 to 30 mbar for 4 h whereafter heating was stopped and the temperature of the solid residue was allowed to decrease slowly to 25° C. at a pressure of from 20 to 30 mbar for 16 h, yielding 0.75 g of crystalline form 7 of sofosbuvir. It was characterized via XRPD analysis according to Reference Example 6. The respectively obtained XRPD pattern is shown in FIG. 7.
RE4.2 Seed crystals of sofosbuvir of polymorphic form 7 were prepared according to the following method: Sofosbuvir of crystalline form 1 (0.095 g, prepared according to WO 2011/123645 A1, example 10) was dissolved in 4 mL 2-octanol upon heating to a temperature in the range of from 50 to 60° C. The hot solution was filtered and allowed to cool to room temperature. Thereafter, sofosbuvir form 6 seed crystals (5 mg, prepared according to WO 2011/123645 A1, example 21) were added. The mixture was allowed to stand at room temperature without mechanical agitation and after one week, crystals deposited at the solution/air boundary. The crystals were collected and air dried.
   5.00 g of sofosbuvir (polymorphic form 1 prepared according to WO 2011/123645 A, Example 10) were dissolved in 50 mL of n-butanol in a 250 mL Erlenmeyer at 40° C. 130 mL of n-heptane were added at 40° C. and the homogeneous solution cooled to room temperature, followed by addition of seeds of polymorphic form 7 (250 mg, prepared as described above in RE4.2). The mixture was slowly shaken (150 r.p.m.) at room temperature. Under these conditions, only the upper part of the solution was in motion. No motion was applied to the lower part of the solution containing the seed crystals, allowing crystal formation on the bottom of the Erlenmeyer. After 90 minutes, the solution was poured into another flask and used for further crystallization process. The resulting solid material formed of the wall of the Erlenmeyer was collected, washed with n-heptane and dried under vacuum at a pressure of from 20 to 30 mbar for 4 h whereafter heating was stopped and the temperature of the solid residue was allowed to decrease slowly to 25° C. at a pressure of from 20 to 30 mbar for 20 h, yielding 2.18 g of crystalline form 7 of sofosbuvir.

### Reference Example 5: Determination of the Moisture Stability of a Polymorphic Form of Sofosbuvir and Amorphous Sofosbuvir

30 mg of a given polymorphic form of sofosbuvir or of amorphous sofosbuvir prepared according to Reference Examples 1 to 4 were analyzed via XRPD as described in Reference Example 6. Then, these polymorphic forms of sofosbuvir or of amorphous sofosbuvir were exposed to an atmosphere having a relative humidity (RH) of 75% and a temperature of 40° C. for 18 hours in a humidity chamber and analyzed via XRPD as described in Reference Example 6. The results obtained are shown in Table 1 below:

TABLE 1
Results of the determination of the moisture stability

| | Sample before storage | | Sample after storage | |
|---|---|---|---|---|
| | at 40° C./75% RH | | at 40° C./75% RH for 18 h | |
| Sample | Visual aspect | XRPD analysis | Visual aspect | XRPD analysis |
| Amorphous | white powder | amorphous | gel/gum*⁾ | amorphous |
| | | (see FIG. 1) | | (see FIG. 2) |
| Form 1 | white powder | crystalline, Form 1 | gel/gum*⁾ | amorphous |
| | | (see FIG. 3) | | (see FIG. 4) |
| Form 6 | white powder | crystalline, Form 6 | white powder | crystalline, Form 6 |
| | | (see FIG. 5) | | (see FIG. 6) |
| Form 7 | white powder | crystalline, Form 7 | white powder | crystalline, Form 7 |
| | | (see FIG. 7) | | (see FIG. 8) |

*⁾Sample deliquesced (formation of a hard, transparent and pasty gel/gum)

### Reference Example 6: Determination of XRPD Patterns

XRPD patterns were obtained with an X'Pert PRO diffractometer (PANalytical, Almelo, The Netherlands) equipped with a theta/theta coupled goniometer in transmission geometry, pro-grammable XYZ stage with well plate holder, Cu-Kalpha_{1,2} radiation source (wavelength 0.15419 nm) with a focusing mirror, a 0.5° divergence slit, a 0.02° soller slit collimator and a 0.5° anti-scattering slit on the incident beam side, a 2 mm anti-scattering slit, a 0.02° soller slit collimator, a Ni-filter and a solid state PIXcel detector on the diffracted beam side. The diffractogram was recorded at room temperature at a tube voltage of 40 kV, tube current of 40 mA, applying a stepsize of 0.013° 2-theta with 40 sec per step in the angular range of 2° to 40° 2-theta. A typical precision of the 2-theta values is in the range of ±0.2° 2-theta. Thus, a diffraction peak that appears for example at 9.4° 2-theta can appear between 9.2 and 9.6° 2-theta on most X-ray diffractometers under standard conditions.

### Reference Example 7: Determination of Characteristics of Polymorphic Form 7

### 7.1 FTIR Spectra

Fourier transform infrared (FTIR) spectra were recorded with a Bruker IFS 25 spectrometer (Bruker Analytische Messtechnik GmbH, Karlsruhe, D) connected to a Bruker IR microscope I (15×-Cassegrain-objective). The samples were prepared on ZnSe-disks and measured at room temperature in transmission mode (spectral range (4000-600) cm⁻¹, resolution 4 cm⁻¹, 64 interferograms per spectrum). To record a spectrum a spatula tip of a sample was applied to the surface of the ZnSe in powder form. Then the sample was pressed onto the ZnSe and the spectrum was recorded. A spectrum of the clean ZnSe was used as background spectrum. A typical precision of the wavenumber values is in the range of about ±2 cm⁻¹. Thus, an infrared peak that appears for example at 1668 cm⁻¹ can appear between 1666 and 1670 cm⁻¹ on most infrared spectrometers under standard conditions.

### 7.2 Melting Point

Differential scanning calorimetry (DSC) was performed with a DSC 7 (Perkin-Elmer, Norwalk, Ct., USA) using a Pyris 2.0 software and with a DSC 204F1 (Netzsch GmbH & Co. Holding AG, Selb, Germany). Approximately (1 to 5±0.005) mg sample (using a UM3 ultramicrobalance, Mettler, Greifensee, CH) was weighed into an Al-pan (30 microL), sealed with a cover, which was perforated by a needle and heated from 25 to 150° C. at a rate of 10 K/min. Dry nitrogen was used as the purge gas (purge: 20 mL/min).

### 7.3 Organic Solvent Content

Thermogravimetric analysis (TGA) was performed using the following equipment/conditions: Thermogravimetric-system TGA-7, Pyris-Software for Windows NT, (Perkin-Elmer, Norwalk, Ct., USA), Platinum-sample holder (50 microL), nitrogen as the purge gas (sample purge: 20 mL/min, balance purge: 40 mL/min). Heating rate: 10 K/min; heating range: 25-145° C.

### 7.4 Water Content

The moisture sorption desorption isotherms were acquired using a SPS-11 moisture sorption analyzer (MD Messtechnik, Ulm, D). The samples were weighed into Aluminium sample holders. The measurement cycles for the novel crystalline form according to the present invention were started at 43% RH, decreased to 40% RH (relative humidity), further decreased in 10% steps to 10% RH, decreased in 5% steps to 0% RH, increased in 5% steps to 10% RH, further increased in 10% steps to 90% RH and subsequently increased to 95% RH, decreased again to 90% RH, decreased in 10% steps to 10% RH, further decreased in 5% steps to 0% RH, again increased in 5% steps to 10%, subsequently increased in 10% steps to 40% RH and finally increased to 43% RH. The measurement cycles for form 1 were started at 43% RH, decreased to 40% RH, further decreased in 10% steps to 10% RH, decreased in 5% steps to 0% RH, increased in 5% steps to 10% RH, further increased in 10% steps to 90% RH and subsequently increased to 91% RH, decreased again to 90% RH, decreased in 10% steps to 10% RH, further decreased in 5% steps to 0% RH, again increased in 5% steps to 10%, subsequently increased in 10% steps to 40% RH and finally increased to 43% RH. The equilibrium condition for each step was set to a mass constancy of ±0.005% over 60 min. The temperature was (25±0.1)° C. The water content of the samples was determined after the moisture sorption/desorption experiments with a TGA 7 system (Perkin Elmer, Norwalk, Conn., USA) using the Pyris 2.0 software. The samples were weighed into Aluminium pans (50 microL). Dry nitrogen was used as purge gas (purge rate: 20 mL/min). The samples were heated from 25 to 200° C. using a heating rate of 10 K/min.

### 7.5 Unit Cell

Intensity data for the crystal structure were recorded at 120 K on a Rigaku AFC12 goniometer driven by the CrystalClear-SM Expert 3.1 b27 software (Rigaku, 2012) and equipped with an enhanced sensitivity (HG) Saturn724+ detector mounted at the window of an FR-E+ Super Bright Mo rotating anode generator (lambda=0.71075 Angstrom) with HFVarimax optics. The structure was solved using the direct methods procedure in SHELXS97 and refined by full-matrix least squares on F² using SHELXL97. All non-hydrogen atoms were refined anisotropically.

### Reference Example 8: Determination of the Mean Dissolution of the Tablets

The dissolution properties of the tablets prepared according to the present invention were determined using a USP dissolution paddle apparatus 2 at 37° C., 75 r.p.m., using a phosphate buffer having a pH of 6.8 and a vessel having a volume of 900 ml. A given obtained dissolution profiles was obtained based on the analysis of one tablet.

### Example 1: Preparation of a Tablet Containing Polymorphic Form 6 of Sofosbuvir

### E1.1 Preparation of a Tablet Having a Sofosbuvir Content of 50 Weight-%

Sofosbuvir of polymorphic form 6 was prepared according to Reference Example 3 above. 2.5 g of polymorphic form 6 were blended with 1.117 g mannitol, 0.919 g microcrystalline cellulose, 0.093 g crosscarmellose sodium, 0.017 g colloidal silica, and 0.028 g magnesium stearate. For blending the compounds, an overhead reax mixture was used. The obtained mixture was compressed under a pressure of 140 bar to obtain flat tablets having a diameter of 15 mm. These tablets were crushed over a sieve having a mesh size of 1 mm. The obtained granulate was admixed with 0.186 g microcrystalline cellulose, 0.093 g crosscarmellose sodium, 0.019 g colloidal silica, and 0.028 g magnesium stearate. For admixing the granulate with the excipients, am overhead reax mixture was used. The obtained mixture was compressed under a pressure of 200 bar to obtain 800 mg oblong tablets of dimensions 18×8 mm. A respectfully prepared tablet had the following composition: 0.400 g polymorphic form 6 of sofosbuvir, 0.179 g mannitol, 0.177 g microcrystalline cellulose, 0.030 g crosscarmellose sodium, 0.006 g colloidal silica, 0.008 g magnesium stearate. In particular, the tablet had the following composition shown in Table 2.1, divided in intragranular and extragranular portion:

TABLE 2.1
Composition of Tablet of Example E1.1

| | |
|---|---|
| Component | content/g |

intragranular

| | |
|---|---|
| sofosbuvir polymorphic form 6 | 0.400 |
| mannitol | 0.179 |
| microcrystalline cellulose (Avicel ®) | 0.147 |
| crosscarmellose sodium (Primojel ®) | 0.015 |
| colloidal silicon dioxide (Aerosil ®) | 0.003 |
| magnesium stearate | 0.004 |

extragranular

| | |
|---|---|
| microcrystalline cellulose (Avicel ®) | 0.030 |
| crosscarmellose sodium (Primojel ®) | 0.015 |
| colloidal silicon dioxide (Aerosil ®) | 0.003 |
| magnesium stearate | 0.004 |

These tablets were subjected to dissolution properties testing according to Reference Example 8 above. The results are described in section “Results of the Examples and the Comparative Examples” below and shown in FIG. 9.

### E1.2 Preparation of a Tablet Having a Sofosbuvir Content of 75 Weight-%

Sofosbuvir of polymorphic form 6 was prepared according to Reference Example 3 above. 3.750 g of polymorphic form 6 were blended with 0.559 g mannitol, 0.459 g microcrystalline cellulose, 0.047 g crosscarmellose sodium, 0.008 g colloidal silica, and 0.014 g magnesium stearate. For blending the compounds, an overhead reax mixture was used. The obtained mixture was compressed under a pressure of 140 bar to obtain flat tablets having a diameter of 15 mm. These tablets were crushed over a sieve having a mesh size of 1 mm. The obtained granulate was admixed with 0.093 g microcrystalline cellulose, 0.047 g crosscarmellose sodium, 0.009 g colloidal silica, and 0.014 g magnesium stearate. For admixing the granulate with the excipients, am overhead reax mixture was used. The obtained mixture was compressed under a pressure of 200 bar to obtain 533 mg oblong tablets of dimensions 18×8 mm. A respectfully prepared tablet had the following composition: 0.4000 g polymorphic form 6 of sofosbuvir, 0.0596 g mannitol, 0.0590 g microcrystalline cellulose, 0.0100 g crosscarmellose sodium, 0.0019 g colloidal silica, 0.0030 g magnesium stearate. In particular, the tablet had the following composition shown in Table 2.2, divided in intragranular and extragranular portion:

TABLE 2.2
Composition of Tablet of Example E1.2

| | |
|---|---|
| Component | content/g |

intragranular

| | |
|---|---|
| sofosbuvir polymorphic form 6 | 0.4000 |
| mannitol | 0.0596 |
| microcrystalline cellulose (Avicel ®) | 0.0490 |
| crosscarmellose sodium (Primojel ®) | 0.0050 |
| colloidal silicon dioxide (Aerosil ®) | 0.0009 |
| magnesium stearate | 0.0015 |

extragranular

| | |
|---|---|
| microcrystalline cellulose (Avicel ®) | 0.0099 |
| crosscarmellose sodium (Primojel ®) | 0.0050 |
| colloidal silicon dioxide (Aerosil ®) | 0.0010 |
| magnesium stearate | 0.0015 |

These tablets were subjected to dissolution properties testing according to Reference Example 8 above. The results are described in section “Results of the Examples and the Comparative Examples” below and shown in FIG. 10.

### E1.3 Preparation of a Tablet Having a Sofosbuvir Content of 60 Weight-%

TABLE 2.2
Composition of Tablet of Example E1.3

| | |
|---|---|
| Component | content/g |

intragranular

| | |
|---|---|
| sofosbuvir polymorphic form 6 | 0.400 |
| mannitol | 0.119 |
| microcrystalline cellulose (Avicel ®) | 0.098 |
| crosscarmellose sodium (Primojel ®) | 0.010 |
| colloidal silicon dioxide (Aerosil ®) | 0.002 |
| magnesium stearate | 0.003 |

extragranular

| | |
|---|---|
| microcrystalline cellulose (Avicel ®) | 0.020 |
| crosscarmellose sodium (Primojel ®) | 0.010 |
| colloidal silicon dioxide (Aerosil ®) | 0.002 |
| magnesium stearate | 0.003 |

The tablet of example E1.3 has been prepared with the same procedure of example E1.2.

### Example 2: Preparation of a Tablet Containing Polymorphic Form 7 of Sofosbuvir

### E2.1 Preparation of a Tablet Having a Sofosbuvir Content of 50 Weight-%

Sofosbuvir of polymorphic form 7 was prepared according to Reference Example RE4.2 above. 2.5 g of polymorphic form 6 were blended with 1.117 g mannitol, 0.919 g microcrystalline cellulose, 0.093 g crosscarmellose sodium, 0.017 g colloidal silica, and 0.028 g magnesium stearate. For blending the compounds, an overhead reax mixture was used. The obtained mixture was compressed under a pressure of 140 bar to obtain flat tablets having a diameter of 15 mm. These tablets were crushed over a sieve having a mesh size of 1 mm. The obtained granulate was admixed with 0.186 g microcrystalline cellulose, 0.093 g crosscarmellose sodium, 0.019 g colloidal silica, and 0.028 g magnesium stearate. For admixing the granulate with the excipients, am overhead reax mixture was used. The obtained mixture was compressed under a pressure of 200 bar to obtain 800 mg oblong tablets of dimensions 18×8 mm. A respectfully prepared tablet had the following composition: 0.400 g polymorphic form 7 of sofosbuvir, 0.179 g mannitol 0.177 g microcrystalline cellulose, 0.030 g crosscarmellose sodium, 0.006 g colloidal silica, 0.008 g magnesium stearate. In particular, the tablet had the following composition shown in Table 3.1, divided in intragranular and extragranular portion:

TABLE 3.1
Composition of Tablet of Example E2.1

| | |
|---|---|
| Component | content/g |

intragranular

| | |
|---|---|
| sofosbuvir polymorphic form 7 | 0.400 |
| mannitol | 0.179 |
| microcrystalline cellulose (Avicel ®) | 0.147 |
| crosscarmellose sodium (Primojel ®) | 0.015 |
| colloidal silicon dioxide (Aerosil ®) | 0.003 |
| magnesium stearate | 0.004 |

extragranular

| | |
|---|---|
| microcrystalline cellulose (Avicel ®) | 0.030 |
| crosscarmellose sodium (Primojel ®) | 0.015 |
| colloidal silicon dioxide (Aerosil ®) | 0.003 |
| magnesium stearate | 0.004 |

These tablets were subjected to dissolution properties testing according to Reference Example 8 above. The results are described in section “Results of the Examples and the Comparative Examples” below and shown in FIG. 9.

### Comparative Example 1: Preparation of a Tablet Containing Polymorphic Form 1 of Sofosbuvir

### CE1.1 Preparation of a Tablet Having a Sofosbuvir Content of 30 Weight-%

Sofosbuvir of polymorphic form 1 was prepared according to Reference Example 2.1 above. 0.995 g of polymorphic form 1 were blended with 0.896 g mannitol, 0.737 g microcrystalline cellulose, 0.075 g crosscarmellose sodium, 0.013 g colloidal silica, and 0.022 g magnesium stearate. For blending the compounds, an overhead reax mixture was used. The obtained mixture was compressed under a pressure of 140 bar to obtain flat tablets having a diameter of 15 mm. These tablets were crushed over a sieve having a mesh size of 1 mm. The obtained granulate was admixed with 0.149 g microcrystalline cellulose, 0.075 g crosscarmellose sodium, 0.015 g colloidal silica, and 0.022 g magnesium stearate. For admixing the granulate with the excipients, an overhead reax mixture was used. The obtained mixture was compressed under a pressure of 200 bar to obtain 800 mg oblong tablets of dimensions 20×9 mm. A respectfully prepared tablet had the following composition: 0.4000 g polymorphic form 1 of sofosbuvir, 0.3600 g mannitol, 0.3561 g microcrystalline cellulose, 0.0600 g crosscarmellose sodium, 0.0114 g colloidal silica, 0.0180 g magnesium stearate. In particular, the tablet had the following composition shown in Table 4.1, divided in intragranular and extragranular portion:

TABLE 4.1
Composition of Tablet of Comparative Example CE1.1

| | |
|---|---|
| Component | content/g |

intragranular

| | |
|---|---|
| sofosbuvir polymorphic form 1 | 0.4000 |
| mannitol | 0.3600 |
| microcrystalline cellulose (Avicel ®) | 0.2961 |
| crosscarmellose sodium (Primojel ®) | 0.0300 |
| colloidal silicon dioxide (Aerosil ®) | 0.054 |
| magnesium stearate | 0.090 |

extragranular

| | |
|---|---|
| microcrystalline cellulose (Avicel ®) | 0.0600 |
| crosscarmellose sodium (Primojel ®) | 0.0300 |
| colloidal silicon dioxide (Aerosil ®) | 0.0060 |
| magnesium stearate | 0.0090 |

These tablets were subjected to dissolution properties testing according to Reference Example 8 above. The results are described in section “Results of the Examples and the Comparative Examples” below and shown in FIG. 9.

### CE1.2 Preparation of a Tablet Having a Sofosbuvir Content of 50 Weight-%

Sofosbuvir of polymorphic form 1 was prepared according to Reference Example 2.2 above. 2.5 g of polymorphic form 1 were blended with 1.117 g mannitol, 0.919 g microcrystalline cellulose, 0.093 g crosscarmellose sodium, 0.017 g colloidal silica, and 0.028 g magnesium stearate. For blending the compounds, an overhead reax mixture was used. The obtained mixture was compressed under a pressure of 140 bar to obtain flat tablets having a diameter of 15 mm. These tablets were crushed over a sieve having a mesh size of 1 mm. The obtained granulate was admixed with 0.186 g microcrystalline cellulose, 0.093 g crosscarmellose sodium, 0.019 g colloidal silica, and 0.028 g magnesium stearate. For admixing the granulate with the excipients, am overhead reax mixture was used. The obtained mixture was compressed under a pressure of 200 bar to obtain 800 mg oblong tablets of dimensions 18×8 mm. A respectfully prepared tablet had the following composition: 0.400 g polymorphic form 1 of sofosbuvir, 0.179 g mannitol, 0.177 g microcrystalline cellulose, 0.030 g crosscarmellose sodium, 0.006 g colloidal silica, 0.008 g magnesium stearate. In particular, the tablet had the following composition shown in Table 4.2, divided in intragranular and extragranular portion:

TABLE 4.2
Composition of Tablet of Comparative Example CE1.2

| | |
|---|---|
| Component | content/g |

intragranular

| | |
|---|---|
| sofosbuvir polymorphic form 1 | 0.400 |
| mannitol | 0.179 |
| microcrystalline cellulose (Avicel ®) | 0.147 |
| crosscarmellose sodium (Primojel ®) | 0.015 |
| colloidal silicon dioxide (Aerosil ®) | 0.003 |
| magnesium stearate | 0.004 |

extragranular

| | |
|---|---|
| microcrystalline cellulose (Avicel ®) | 0.030 |
| crosscarmellose sodium (Primojel ®) | 0.015 |
| colloidal silicon dioxide (Aerosil ®) | 0.003 |
| magnesium stearate | 0.004 |

These tablets were subjected to dissolution properties testing according to Reference Example 8 above. The results are described in section “Results of the Examples and the Comparative Examples” below and shown in FIG. 9.

### Comparative Example 2: Preparation of a Tablet Containing Amorphous Sofosbuvir

### CE2.1 Preparation of a Tablet Having a Sofosbuvir Content of 50 Weight-%

Amorphous sofosbuvir was prepared according to Reference Example RE1.2 above. 2.5 g of amorphous sofosbuvir were blended with 1.117 g mannitol, 0.919 g microcrystalline cellulose, 0.093 g crosscarmellose sodium, 0.017 g colloidal silica, and 0.028 g magnesium stearate. For blending the compounds, an overhead reax mixture was used. The obtained mixture was compressed under a pressure of 140 bar to obtain flat tablets having a diameter of 15 mm. These tablets were crushed over a sieve having a mesh size of 1 mm. The obtained granulate was admixed with 0.186 g microcrystalline cellulose, 0.093 g crosscarmellose sodium, 0.019 g colloidal silica, and 0.028 g magnesium stearate. For admixing the granulate with the excipients, am overhead reax mixture was used. The obtained mixture was compressed under a pressure of 200 bar to obtain 800 mg oblong tablets of dimensions 18×8 mm. A respectfully prepared tablet had the following composition: 0.400 g amorphous sofosbuvir, 0.179 g mannitol, 0.177 g microcrystalline cellulose, 0.030 g crosscarmellose sodium, 0.006 g colloidal silica, 0.008 g magnesium stearate. In particular, the tablet had the following composition shown in Table 5.1, divided in intragranular and extragranular portion:

TABLE 5.1
Composition of Tablet of Comparative Example CE2.1

| | |
|---|---|
| Component | content/g |

intragranular

| | |
|---|---|
| amorphous sofosbuvir | 0.400 |
| mannitol | 0.179 |
| microcrystalline cellulose (Avicel ®) | 0.147 |
| crosscarmellose sodium (Primojel ®) | 0.015 |
| colloidal silicon dioxide (Aerosil ®) | 0.003 |
| magnesium stearate | 0.004 |

extragranular

| | |
|---|---|
| microcrystalline cellulose (Avicel ®) | 0.030 |
| crosscarmellose sodium (Primojel ®) | 0.015 |
| colloidal silicon dioxide (Aerosil ®) | 0.003 |
| magnesium stearate | 0.004 |

These tablets were subjected to dissolution properties testing according to Reference Example 8 above. The results are described in section “Results of the Examples and the Comparative Examples” below and shown in FIG. 9.

### Results of the Examples and the Comparative Examples

As shown in FIG. 9, a low drug load tablet according to the prior art, having a content of polymorphic form 1 of sofosbuvir of 30 weight-%, shows a good dissolution profile up to about 5 minutes.

A respective high drug load tablet comprising said polymorphic form 1 of sofosbuvir having a drug content of 50 weight-%, however, shows a very disadvantageous dissolution profile, as also depicted in FIG. 9. Such a disadvantageous dissolution profile is also obtained when a high drug load tablet is prepared, containing 50 weight-% of amorphous sofosbuvir; again, reference is made to the respective graph in FIG. 9.

Surprisingly, high drug load tablets with a drug content of 50 weight-% were obtained when a polymorphic form of sofosbuvir was used as drug, which polymorphic form has a moisture stability of at least 95%. This general concept is shown for polymorphic form 6 and polymorphic form 7 of sofosbuvir; reference is made to FIG. 9. Even more surprisingly, as can be taken from FIG. 9, the dissolution profile of said high drug load tablets containing 50 weight-% of a polymorphic form having a moisture stability of at least 95% is advantageous even compared to a low drug load tablet according to the prior art for higher dissolution times, starting, for polymorphic form 7, at a dissolution time of about 10 minutes, for polymorphic form 6 at a dissolution time of about 15 minutes.

Also for high drug load tablets with a drug content of 60 or 75 weight-%, the advantageous properties described above were obtained. For example, FIG. 11 and FIG. 10 show a high drug load tablet comprising polymorphic form 6 of sofosbuvir in an amount of 60 and 75 weight-%. The very advantageous tablets dissolution properties as already observed for the respective 50 weight-% tablet were obtained.

### BRIEF DESCRIPTION OF THE FIGURES

FIG. 1 shows the XRPD pattern, obtained via XRPD analysis according to Reference Example 6, of amorphous sofosbuvir prepared according to Reference Example 1. The x axis shows 2 theta values in °, with tick marks at 5, 10, 15, 20, 25, 30, 35. The y axis shows the intensity in counts, with tick marks at 0, 100, 200, 300, 400, 500.

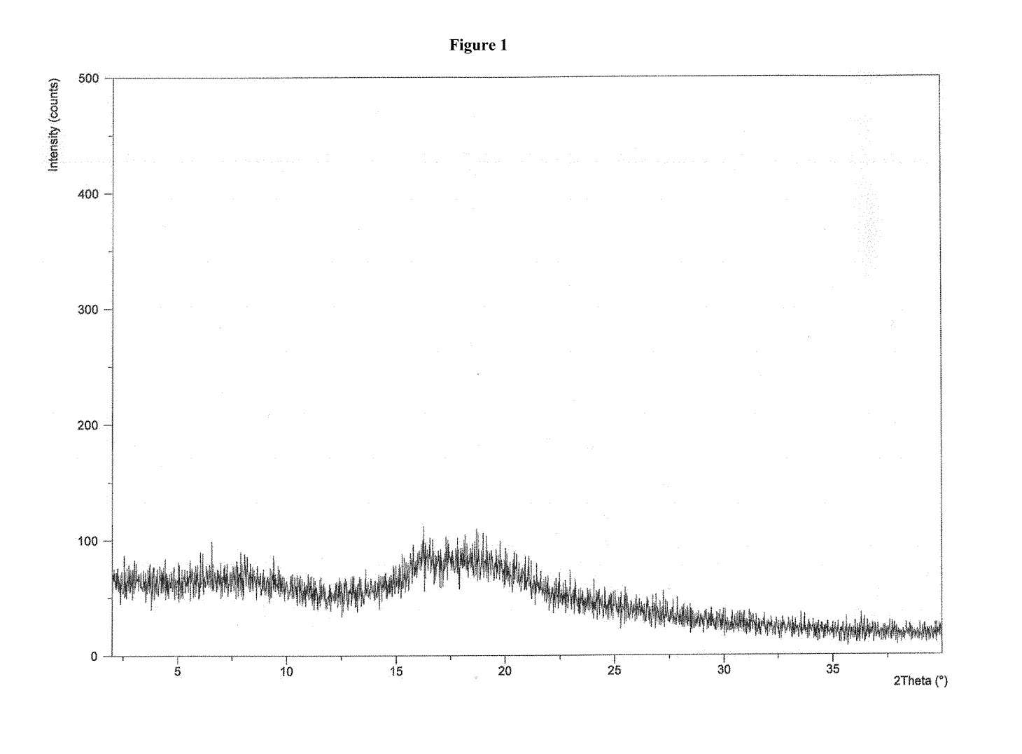

FIG. 2 shows the XRPD pattern, obtained via XRPD analysis according to Reference Example 6, of sofosbuvir prepared according to Reference Example 1 after exposure to humidity according to Reference Example 5. The x axis shows 2 theta values in °, with tick marks at 5, 10, 15, 20, 25, 30, 35. The y axis shows the intensity in counts, with tick marks at 0, 100, 200, 300, 400, 500.

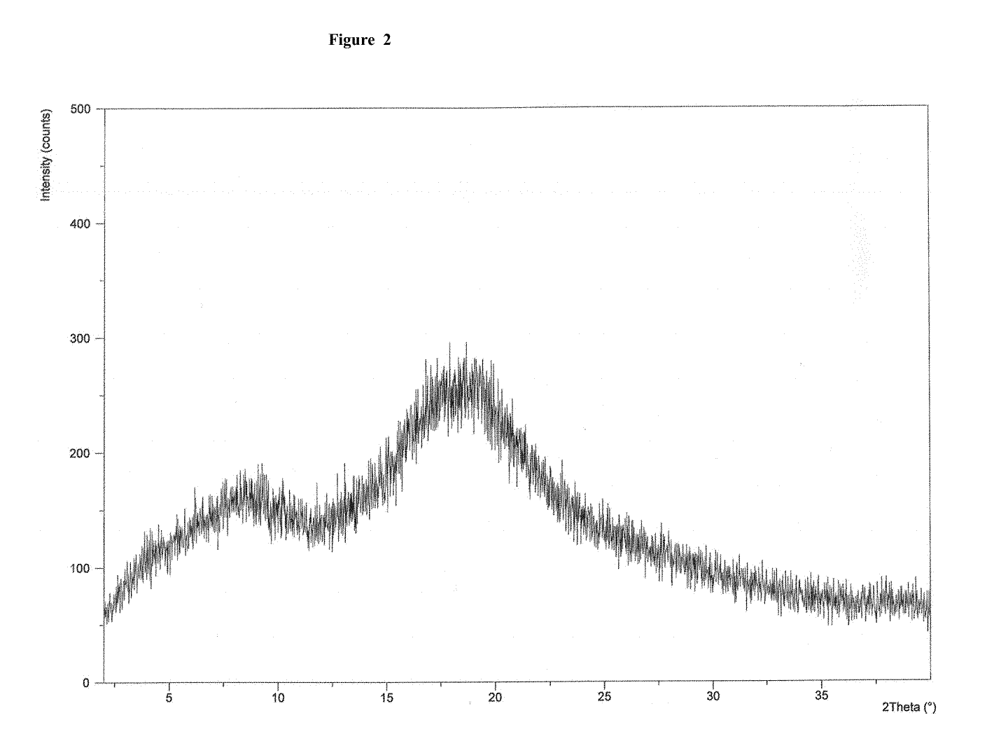

FIG. 3 shows the XRPD pattern, obtained via XRPD analysis according to Reference Example 6, of sofosbuvir of polymorphic form 1 prepared according to Reference Example 2. The x axis shows 2 theta values in °, with tick marks at 5, 10, 15, 20, 25, 30, 35. The y axis shows the intensity in counts, with tick marks at 0, 1000, 2000, 3000, 4000.

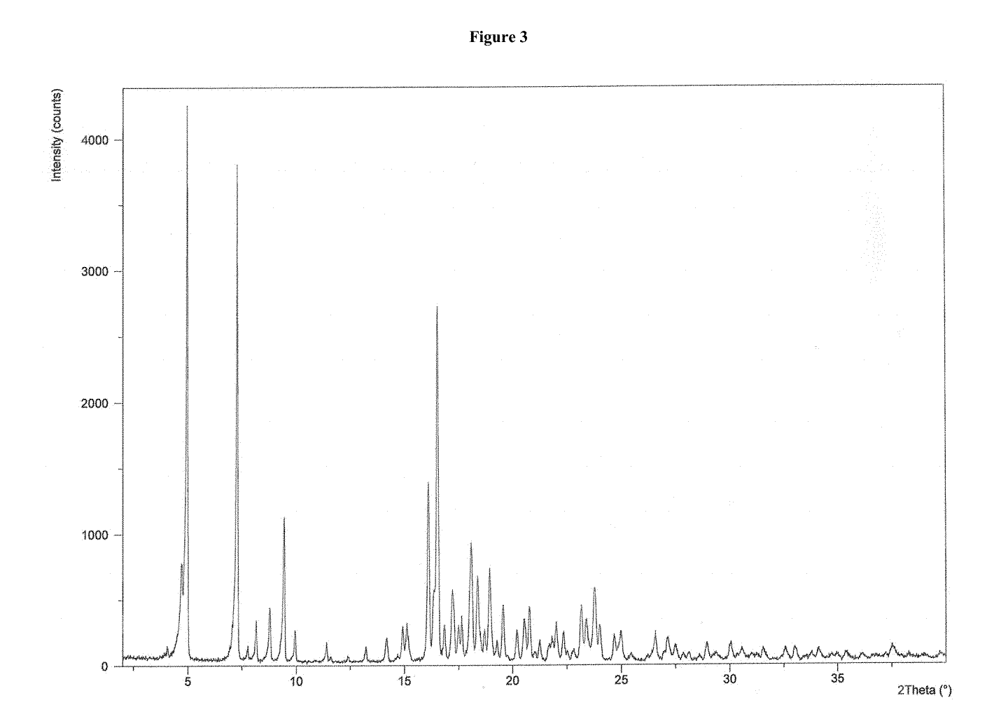

FIG. 4 shows the XRPD pattern, obtained via XRPD analysis according to Reference Example 6, of sofosbuvir prepared according to Reference Example 2 after exposure to humidity according to Reference Example 5. The x axis shows 2 theta values in °, with tick marks at 5, 10, 15, 20, 25, 30, 35. The y axis shows the intensity in counts, with tick marks at 0, 100, 200, 300, 400, 500.

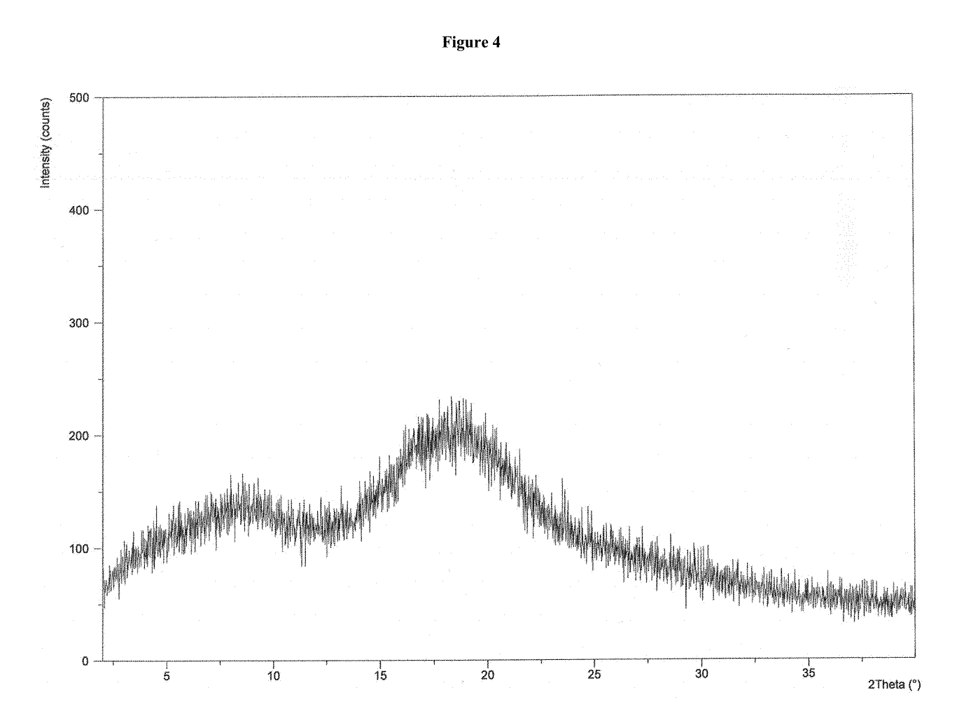

FIG. 5 shows the XRPD pattern, obtained via XRPD analysis according to Reference Example 6, of sofosbuvir of polymorphic form 6 prepared according to Reference Example 3. The x axis shows 2 theta values in °, with tick marks at 5, 10, 15, 20, 25, 30, 35. The y axis shows the intensity in counts, with tick marks at 0, 200, 400, 600, 800.

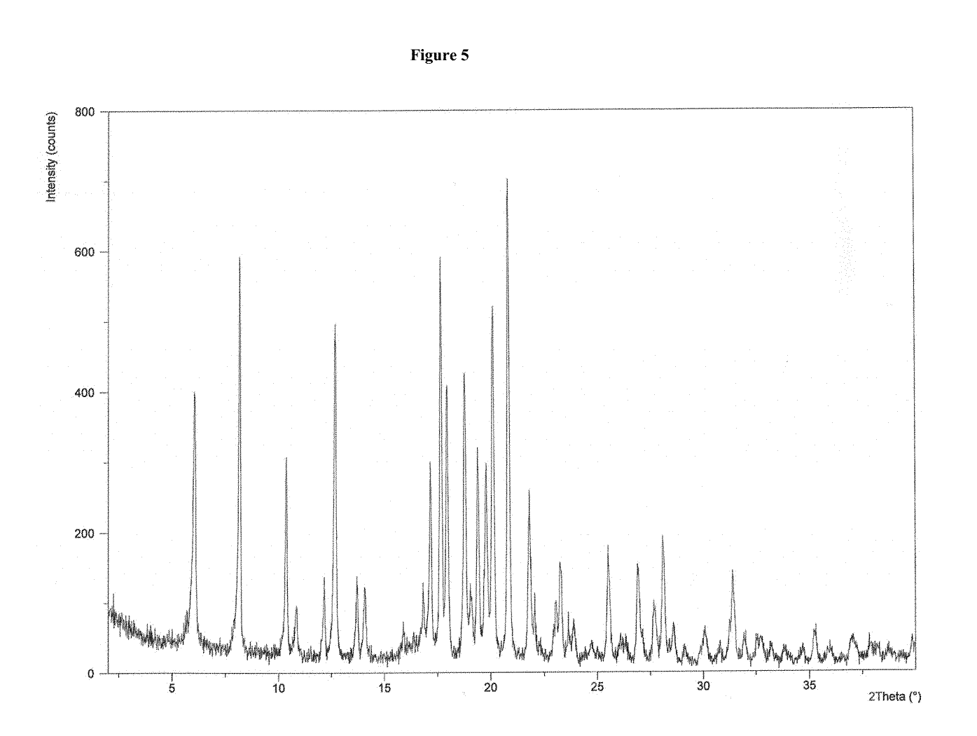

FIG. 6 shows the XRPD pattern, obtained via XRPD analysis according to Reference Example 6, of sofosbuvir prepared according to Reference Example 3 after exposure to humidity according to Reference Example 5. The x axis shows 2 theta values in °, with tick marks at 5, 10, 15, 20, 25, 30, 35. The y axis shows the intensity in counts, with tick marks at 0, 200, 400, 600, 800, 1000, 1200.

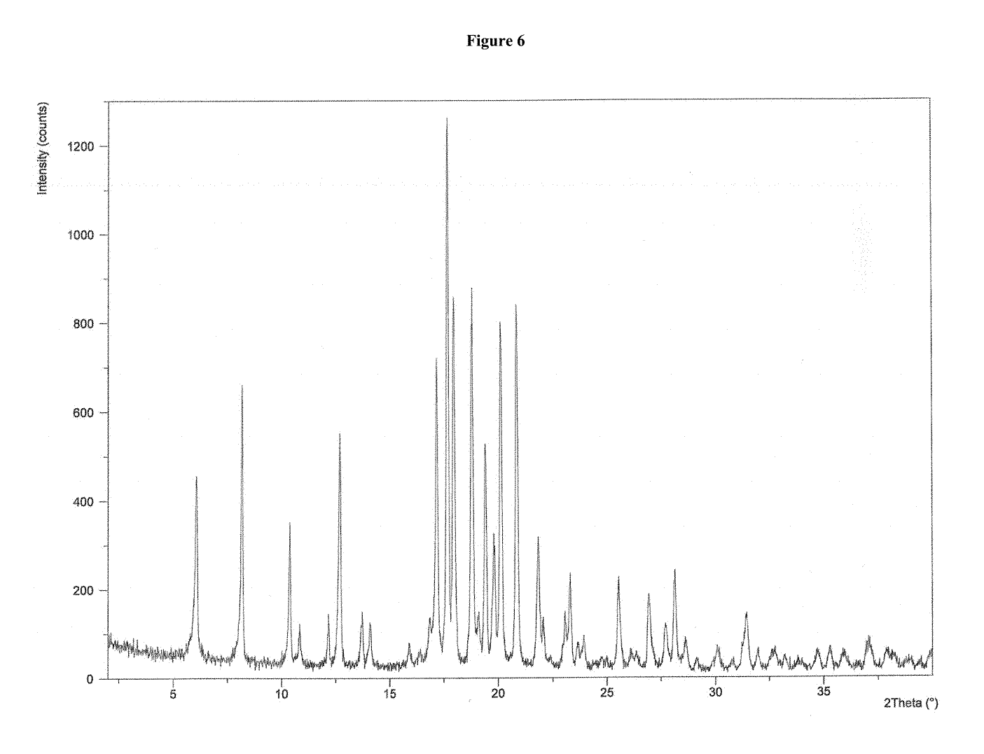

FIG. 7 shows the XRPD pattern, obtained via XRPD analysis according to Reference Example 6, of sofosbuvir of polymorphic form 7 prepared according to Reference Example 4. The x axis shows 2 theta values in °, with tick marks at 5, 10, 15, 20, 25, 30, 35. The y axis shows the intensity in counts, with tick marks at 0, 2000, 4000, 6000.

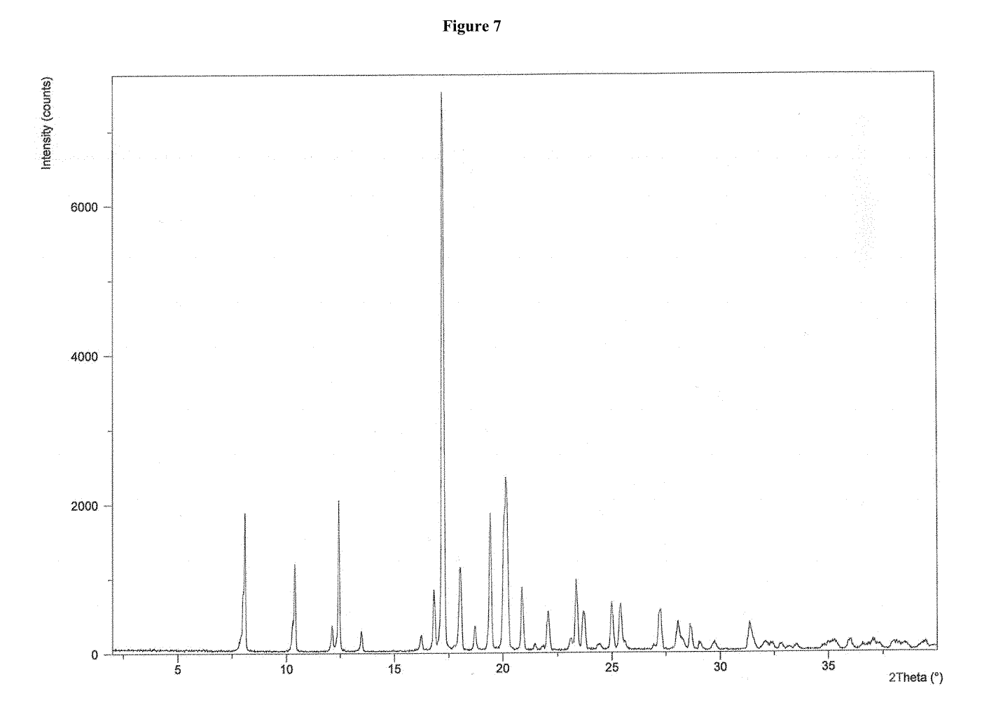

FIG. 8 shows the XRPD pattern, obtained via XRPD analysis according to Reference Example 6, of sofosbuvir prepared according to Reference Example 4 after exposure to humidity according to Reference Example 5. The x axis shows 2 theta values in °, with tick marks at 5, 10, 15, 20, 25, 30, 35. The y axis shows the intensity in counts, with tick marks at 0, 500, 1000, 1500, 2000, 2500.

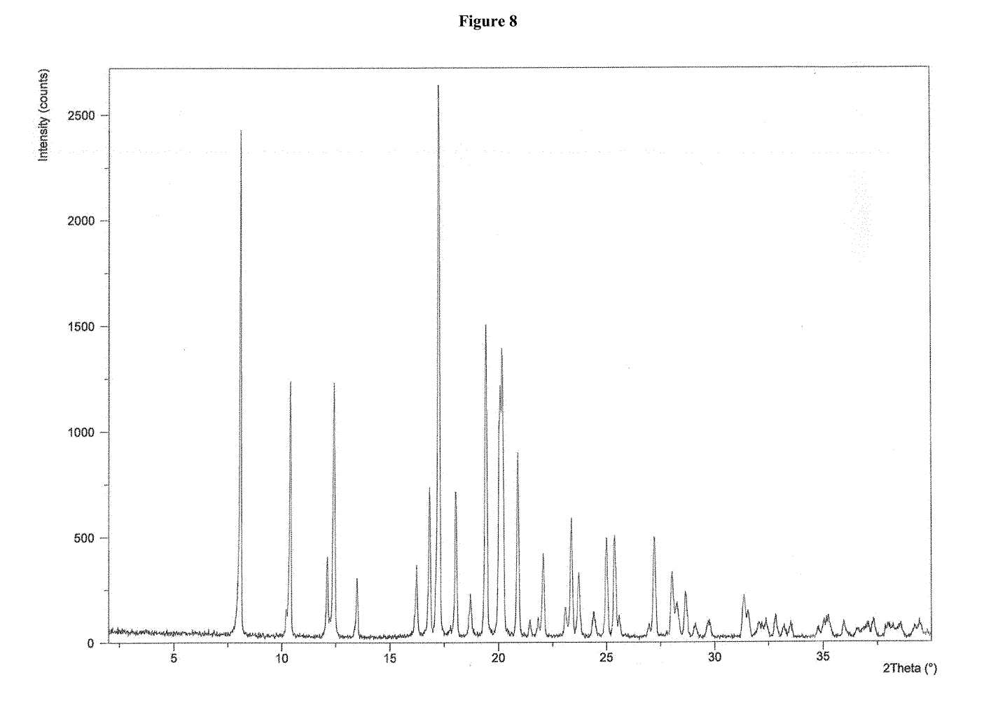

FIG. 9 shows the dissolution profiles obtained according to the method as described in Reference Example 8 hereinabove of tablets having a sofosbuvir content of 30/50 weight-%. The x axis shows the time for which the tablets were subjected to dissolution testing, the y axis shows the dissolution in %. The x axis has tick marks at 0 min, 5 min, 10 min, 15 min, 20 min, 25 min, 30 min. The x axis has tick marks at 0.0, 10.0, 20.0, 30.0, 40.0, 50.0, 60.0, 70.0, 80.0, 90.0, 100.0.

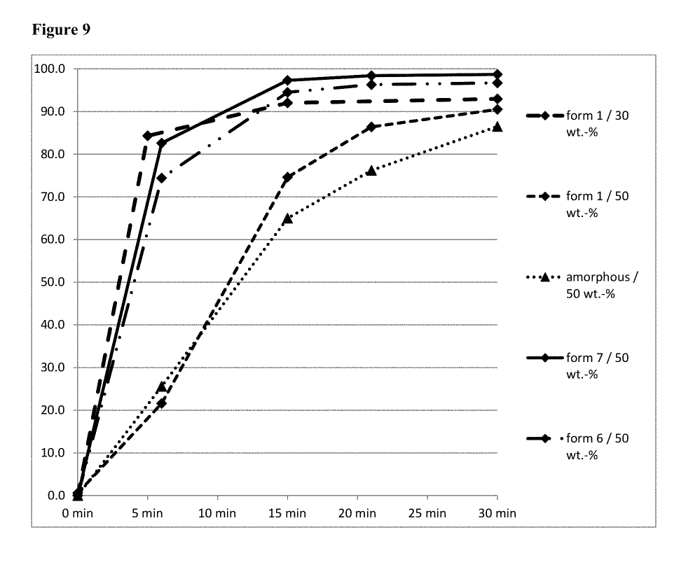

FIG. 10 shows the dissolution profiles obtained according to the method as described in Reference Example 8 hereinabove of a tablet having a sofosbuvir polymorphic form 6 content of 75 weight-%. The x axis shows the time for which the tablets were subjected to dissolution testing, the y axis shows the dissolution in %. The x axis has tick marks at 0 min, 5 min, 10 min, 15 min, 20 min, 25 min, 30 min. The x axis has tick marks at 0.0, 10.0, 20.0, 30.0, 40.0, 50.0, 60.0, 70.0, 80.0, 90.0, 100.0.

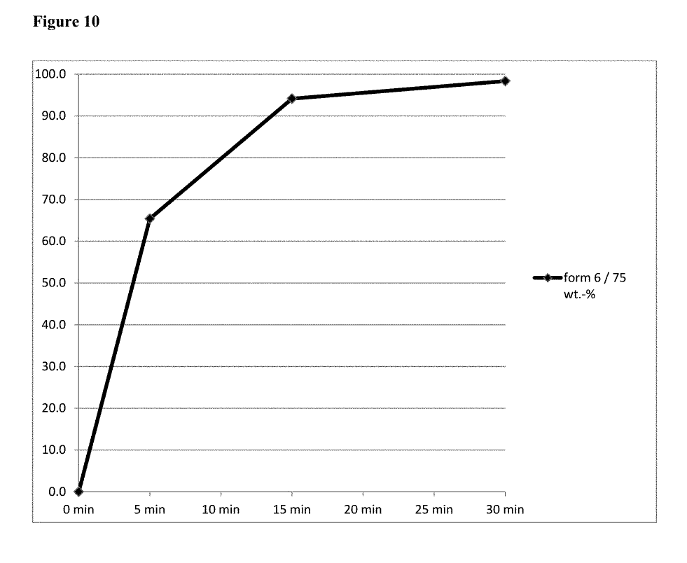

FIG. 11 shows the dissolution profiles obtained according to the method as described in Reference Example 8 hereinabove of a tablet having a sofosbuvir polymorphic form 6 content of 60 weight-%. The x axis shows the time for which the tablets were subjected to dissolution testing, the y axis shows the dissolution in %. The x axis has tick marks at 0 min, 10 min, 20 min, 30 min, 40 min, 50 min, 60 min and 70 min. The x axis has tick marks at 0.0, 20.0, 40.0, 60.0, 80.0, 100.0 and 120.0.

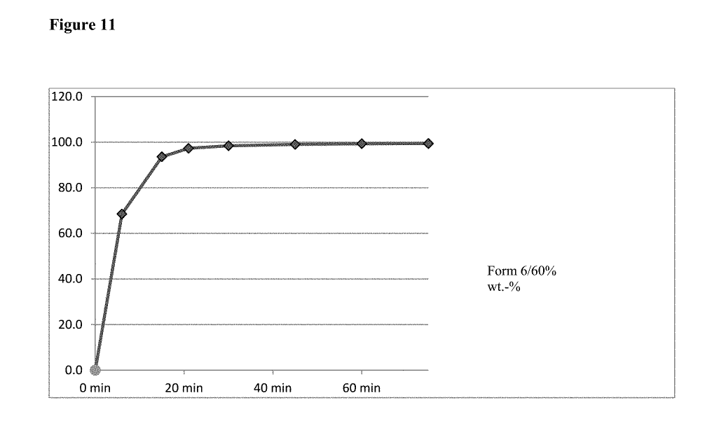

### CITED LITERATURE

WO 2010/135569 A
WO 2011/123645 A
WO 2013/082003 A

1-2. (canceled)

3. A tablet comprising a polymorphic form of crystalline sofosbuvir according to formula (I) 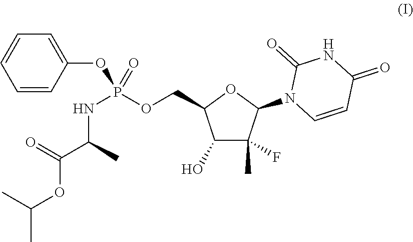 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]
in an amount of at least 25 weight-%, based on the total weight of the tablet, wherein the polymorphic form has a moisture stability of at least 95%, wherein the moisture stability of the polymorphic form is defined as the amount of the polymorphic form which is present after an exposure to a relative humidity of (75±1) % at a temperature of (40±1)° C. for an exposure time of (18±1) hours, relative to the amount of the polymorphic form before said exposure, wherein the polymorphic form is polymorphic form 7 characterized by having an X-ray powder diffraction pattern comprising no reflection at 2-theta angles in the range of from 2.0 to 7.8°, the X-ray powder diffraction pattern is when measured at a temperature in the range of from 15 to 25° C. with Cu-Kalpha_{1,2} radiation having a wavelength of 0.15419 nm.

4. The tablet of claim 3, wherein the polymorphic form is a combination of polymorphic form 6 and polymorphic form 7.

5. (canceled)

6. The tablet of claim 3, comprising the polymorphic form in an amount of ≧75 weight-% based on the total weight of the tablet, wherein the polymorphic form is form 7.

7-10. (canceled)

11. The tablet of claim 3, further comprising at least one pharmaceutically acceptable excipient.

12. The tablet of claim 11, wherein at least 95 weight-% of the tablet consist of the polymorphic form of crystalline sofosbuvir according to formula (I) and the at least one pharmaceutically acceptable excipient.

13. The tablet of claim 11, wherein at least 95 weight-% of the tablet consists of the polymorphic form of crystalline sofosbuvir according to formula (I) and the at least one pharmaceutically acceptable excipient.

14. The tablet of claim 11, wherein at least 99.9 weight-% of the tablet consists of the polymorphic form of crystalline sofosbuvir according to formula (I) and the at least one pharmaceutically acceptable excipient.

15-16. (canceled)

17. The tablet of claim 11, wherein the at least one excipient comprises a combination of at least one diluent and at least one disintegrant and at least one glidant and at least one lubricant, and wherein the weight ratio of the at least one disintegrant relative to the at least diluent is in the range of from 0.01:1 to 0.5:1, the weight ratio of the at least one glidant relative to the at least diluent is in the range of from 0.001:1 to 0.1:1, and the weight ratio of the at least one lubricant relative to the at least diluent is in the range of from 0.005:1 to 0.2:1.

18. The tablet of claim 17, wherein the at least one diluent comprises a combination of mannitol and microcrystalline cellulose, the at least one disintegrant comprises croscarmellose sodium, the at least one glidant comprises colloidal silicon dioxide, and the at least one lubricant comprises magnesium stearate.

19. The tablet of claim 3, having a mass in the range of from 0.45 to 1.00 g and a longest dimension of at most 20 mm.

20. The tablet of claim 3, exhibiting a dissolution of at least 40% after 5 minutes; a dissolution of at least 60% after 10 minutes, and a dissolution of at least 80% after 15 minutes in the mean dissolution profile as determined with a USP dissolution paddle apparatus 2 at 37° C., 75 r.p.m., using a phosphate buffer having a pH of 6.8 and a vessel having a volume of 900 ml.

21. A process for preparing a tablet according to claim 3, comprising at least one pharmaceutically acceptable excipient, said process comprising
(a) providing the polymorphic form of crystalline sofosbuvir according to formula (I), 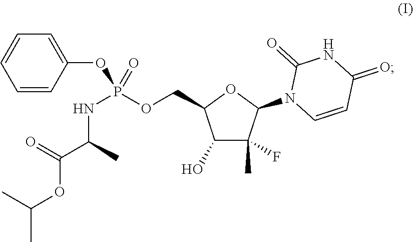 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]
(b) blending the polymorphic form provided in (a) with at least one pharmaceutically acceptable excipient;
(c) preparing the tablet based on the blend obtained in (b).

22. The process of claim 21, wherein the blending according to (b) is carried out by dry granulation.

23. The process of claim 21, wherein (b) comprises
(b.1) blending the polymorphic form provided in (a) with a first portion of the at least one pharmaceutically acceptable excipient;
(b.2) compressing the blend obtained in (b.1), obtaining a molding;
(b.3) crushing the molding, obtained in (b.2);
(b.4) blending the particles obtained in (b.3) with a second portion of the at least one pharmaceutically acceptable excipient;
and wherein (c) comprises
(c) compressing the blend obtained in (b.4), obtaining the tablet.

24-25. (canceled)

26. The process of claim 21, wherein the tablet comprises the polymorphic form in an amount ≧75 weight-%, based on the total weight of the tablet.

27. The process of claim 26, wherein the tablet comprises the polymorphic form in an amount of at least 25 weight based on the total weight of the tablet.

28. The process of claim 21, wherein the polymorphic form is a combination of polymorphic form 6 and polymorphic form 7.

29. The process of claim 21, wherein at least 95 weight-% of the tablet consist of the polymorphic form of crystalline sofosbuvir according to formula (I) and the at least one pharmaceutically acceptable excipient.

30. The process of claim 21, wherein at least 99.9 weight-% of the tablet of (c) consist of the polymorphic form of crystalline sofosbuvir according to formula (I) and the at least one pharmaceutically acceptable excipient.

31. (canceled)

32. A method for treating hepatitis C comprising administering a tablet according to claim 3 to a human in need thereof.
